(19) United States
(12) Patent Application Publication
(10) Pub. No.: US 2023/0285498 A1
(43) Pub. Date: Sep. 14, 2023

(54) TREATMENTS FOR CANCERS HAVING KRAS MUTATIONS

(71) Applicant: City of Hope, Duarte, CA (US)

(72) Inventors: Prakash Kulkarni, Duarte, CA (US); Ravi Salgia, Duarte, CA (US); Atish Mohanty, Duarte, CA (US)

(21) Appl. No.: 18/017,982

(22) PCT Filed: Aug. 5, 2021
(86) PCT No.: PCT/US2021/044711

§ 371 Date: Jan. 25, 2023

Related U.S. Application Data
(60) Provisional application No. 63/062,628, filed on Aug. 7, 2020.

(51) Int. Cl. A61K 38/07; A61K 31/095; A61K 31/519; A61P 35/00; A61K 45/06

(52) U.S. Cl. CPC A61K 38/07; A61K 31/095; A61K 31/519; A61P 35/00; A61K 45/06

(57) ABSTRACT

Provided herein are, inter alia, methods for treating cancers having KRAS mutations using ITGB4/PXN pathway inhibitors, Wnt/β-catenin pathway inhibitors, KRAS pathway inhibitors, and combinations thereof; and pharmaceutical compositions comprising the ITGB4/PXN pathway inhibitors, Wnt/β-catenin pathway inhibitors, KRAS pathway inhibitors, and combinations thereof.

### CROSS-REFERENCE TO RELATED APPLICATIONS

This application claims priority to U.S. Application No. 63/062,628 filed Aug. 7, 2020, the disclosure of which is incorporated by reference herein in its entirety.

### BACKGROUND

Lung cancer is the most frequently diagnosed cancer and a leading cause of cancer-related death worldwide making up almost 25% of all cancer deaths. In 2021, about 235,760 new cases and about 131,880 deaths from lung cancer are expected in the US alone. (Ref 1). Non-small cell lung cancer (NSCLC) is the most commonly diagnosed form of the disease, accounting for more than 85% of cases. (Ref 2). NSCLC can be subdivided into several histologies (adenocarcinoma, squamous cell carcinoma, and large cell carcinoma) and molecular aberrations including KRAS. (Ref 3). In fact, KRAS mutation is the most common gain-of-function alteration accounting for about 30% of lung adenocarcinomas (LUAD) in western countries. (Refs. 4-5).

Although multiple point mutations exist within KRAS, the majority affect codon 12 with the remaining mostly centered on positions 13 and 61. (Ref 5). Functionally, these point mutations result in amino acid substitutions that impair the GTPase activity of KRAS and render the oncoprotein constitutively active. Mutant KRAS with different amino acid substitutions also associate with distinct biological behavior leading to different clinical outcomes. (Refs. 6-9). For example, in NSCLC, tumors with KRAS G12C exhibited higher ERK1/2 phosphorylation than those with KRAS G12D. (Ref 10). Indeed, KRAS G12C tumors were significantly more sensitive to MEK inhibitors than KRAS G12D tumors, and inhibiting MEK in these animals significantly increased chemotherapeutic efficacy and progression-free survival (PFS) of the KRAS G12C mice. (Ref 10). Using functional genomics that identified pathway rewiring in lung and pancreatic cancer cells treated with yet another KRAS G12C inhibitor ARS1620, found that co-inhibiting the phosphatase SHP2 or the receptor kinases EGFR or FGFR enhanced the drug's target engagement, and that inhibiting the kinases AXL, PI3K, or CDK4/6 suppressed alternative pathways of tumor cell survival. (Ref 53). Similarly, in NSCLC, identified bypass mechanisms in subsets of quiescent cells. (Ref 54). The authors found that targeting key proteins, including EGFR and SHP2 as well as the kinase AURKA, prevented these pathways from driving new synthesis of KRAS G12C and consequently reactivating KRAS signaling. Thus, co-treatment with inhibitors of some of the bypass pathways can abrogate the adaptive response of cancer cells to KRAS inhibitors, resulting in greater suppression of tumor growth inhibition. Together, these observations reveal the complexity of the molecular mechanisms underlying drug resistance in KRAS mutant NSCLC while highlighting the potential for combination treatments in alleviating KRAS G12C inhibitor resistance.

Consistent with these observations, Molecular Dynamics Simulations (MDS) studies showed that KRAS is a hybrid protein with several intrinsically disordered regions interdigitated between highly ordered regions and that, different conformations imposed by distinct KRAS oncogene substitutions could lead to altered association with downstream signaling transducers. (Refs. 11-15). Of note, these mutations also impact patient prognosis and are negatively associated with response to targeted therapy; Papadimitrakopoulou et al, 2016 and chemotherapy. (Refs. 16-19).

Until recently, KRAS was considered ‘undruggable’. Thus, in the past two decades, drug discovery efforts were largely focused on targeting actionable mutations. (Refs. 20-21). However, over the past few years, highly efficacious mutant selective KRAS G12C inhibitors namely sotorasib (LUMAKRAS™ by Amgen Inc.) and adagrasib that covalently bind to the cysteine residue have been developed. (Refs. 22-26). However, both these inhibitors bind the GDP-bound, inactive state, demonstrating that KRAS G12C is not constitutively active as previously thought but is indeed hyperexcitable. Furthermore, other inhibitors such as MRTX1133 (Mirati) that is designed to specifically target KRAS G12D and a pan-KRAS inhibitor BI 1701963 (Boehringer-Ingelheim) to target the interactions between RAS and either its downstream effectors or SOS, a guanine-nucleotide-exchange factor that activates RAS are also on the horizon giving new hope to patients with KRAS mutant NSCLC. Although these new drugs are showing promising results in the clinic, they are only partially effective (progression-free survival is 6.3 months with sotorasib), and resistance, especially acquired resistance involving genetic and non-genetic mechanisms, has emerged as a serious concern in NSCLC. (Refs. 24, 26-31, 53).

There is a need in the art for drugs and treatment regimens that overcome the problems of drug resistance in cancers. The disclosure is directed to this, as well as other, important ends.

### BRIEF SUMMARY

The disclosure provides methods of treating cancer in a subject by administering an effective amount of an ITGB4/PXN pathway inhibitor and an effective amount of a KRAS inhibitor; wherein the cancer has a KRAS mutation. In aspects, the methods further comprise administering an effective amount of a Wnt/β-catenin pathway inhibitor.

The disclosure provides methods of treating cancer in a subject by administering an effective amount of a Wnt/β-catenin pathway inhibitor and an effective amount of a KRAS inhibitor; wherein the cancer has a KRAS mutation.

The disclosure provides methods to select a subject with cancer for treatment with a ITGB4/PXN pathway inhibitor and a KRAS inhibitor by measuring a KRAS mutation in a biological sample obtained from the subject; wherein if the KRAS mutation is present in the biological sample, the subject is selected for treatment with the ITGB4/PXN pathway inhibitor and the KRAS inhibitor.

The disclosure provides methods to select a subject with cancer for treatment with a Wnt/β-catenin pathway inhibitor and a KRAS inhibitor by measuring a KRAS mutation in a biological sample obtained from the subject; wherein if the KRAS mutation is present in the biological sample, the subject is selected for treatment with the ITGB4/PXN inhibitor and the KRAS inhibitor.

The disclosure provides methods of detecting resistance to cancer treatment with a KRAS inhibitor in a subject in need thereof, the method comprising: (i) measuring the expression level of ITGB4, PXN, Wnt, β-catenin, or a combination of two or more thereof in a biological sample obtained from the subject; (ii) comparing the expression level of ITGB4, PXN, Wnt, β-catenin, or a combination of two or more thereof in the biological sample obtained from the subject to the expression level of ITGB4, PXN, Wnt, β-catenin, or a combination of two or more thereof in a control; wherein an elevated expression level of ITGB4, PXN, Wnt, β-catenin, or a combination of two or more thereof indicates the subject is resistant to treatment with the KRAS inhibitor. In aspects, the methods further comprise administering to the subject an effective amount of an ITGB4/PXN pathway inhibitor and a KRAS inhibitor when the subject has an elevated expression level of ITGB4, PXN, or both. In aspects, the methods further comprise administering to the subject an effective amount of an Wnt/β-catenin pathway inhibitor and a KRAS inhibitor when the subject has an elevated expression level of ITGB4, PXN, or both. In aspects, the methods further comprise administering to the subject an effective amount of an ITGB4/PXN pathway inhibitor, Wnt/β-catenin pathway inhibitor, and a KRAS inhibitor when the subject has an elevated expression level of ITGB4, PXN, or both.

The disclosure provides methods to treat cancer in a subject in need thereof, the method comprising: (i) identifying a homozygous KRAS mutation in a biological sample obtained from the subject; (ii) administering an ITGB4/PXN inhibitor and a KRAS inhibitor to the subject having the homozygous KRAS mutation. In aspects, the methods further comprise administering an effective amount of a Wnt/β-catenin pathway inhibitor. In embodiments, the disclosure provides methods to treat cancer in a subject in need thereof, the method comprising: (i) identifying a homozygous KRAS mutation in a biological sample obtained from the subject; (ii) administering a Wnt/β-catenin pathway inhibitor and a KRAS inhibitor to the subject having the homozygous KRAS mutation.

These and other embodiments and aspects of the disclosure are provided herein.

### BRIEF DESCRIPTION OF THE DRAWINGS

FIGS. 1A-1E illustrate NSCLC KRAS G12C cell lines that respond to sotorasib at varying concentrations. FIG. 1A: NSCLC cell lines (H358, H23, SW1573) with a KRAS G12C mutation were treated with an increasing concentration (0.3-10 μM) of sotorasib, and fold change in cell count was determined over the course of 72 hours. FIG. 1B: H358, H23, and SW1573 cell line-derived spheroids were treated with an increasing concentration (0.3-10 μM) of sotorasib, and images were taken with the IncuCyte live cell imaging system on day 5. Red fluorescence (primarily seen at 0 μM in all cell lines and in the center of SW1573 cell line at all concentrations) indicates cell viability and green fluorescence (primarily seen surrounding the center in H23 cell line at concentrations of 0.6 μM or greater, with some green fluorescence shown at 0.3 μM) indicates caspase-3/7 activity. FIGS. 1C-1D: Spheroid area and red fluorescence intensity were determined over the course of 120 h. FIG. 1E: Caspase-3/7 activity was measured over the course of 120 hours.

FIGS. 2A-2G illustrate NSCLC KRAS G12C cell lines that respond to KRAS G12C inhibitors, sotorasib and ARS1620 at varying concentrations. FIG. 2A: depicts IC50 values for H23, H358, and SW1573 cell lines, when treated with sotorasib at 72 hours. FIG. 2B: H358, H23, SW1573 were treated with an increasing concentration (0.3-10 μM) of ARS1620, and fold change in cell count was determined over the course of 72 hours. FIG. 2C: ARS1620 IC50 values for H23, H358, and SW1573 cell lines at 72 hours. FIG. 2D: H358, H23, and SW1573 cell line-derived spheroids were treated with an increasing concentration (0.3-10 μM) of ARS1620, and images were taken with IncuCyte live cell imaging system on day 5. Red fluorescence indicates cell viability and green fluorescence indicates caspase-3/7 activity. FIGS. 2E-2F: Spheroid area and red fluorescence intensity were determined over the course of 120 h. FIG. 2G: Caspase-3/7 activity was measured over the course of 120 hours.

FIGS. 3A-3F illustrate inhibition of ITGB4/PXN axis with siRNA or carfilzomib that sensitizes cells to sotorasib treatment. FIG. 3A: H23, H358, and SW1573 cells were treated with sotorasib IC50 concentration for 72 hours, and changes in protein expression and signaling were determined by immunoblot. FIG. 3B: Effect of PXN and ITGB4 single knockdowns and double knockdown with sotorasib on cell proliferation and caspase activity after 72 hours. FIG. 3C: Immunoblot confirmed knockdown with ITGB4 and PXN siRNA and effect of addition of ARS1620 and sotorasib on protein expression and signaling after 72 hours. FIG. 3D: H23 cells with ITGB4 overexpression (OE) were treated with an increasing concentration (3-9 μM) of sotorasib for 72 hours to determine effect on protein expression and signaling with immunoblot. FIG. 3E: SW1573 cells were treated with 8 different concentrations of sotorasib and carfilzomib in the form of a matrix to determine percentage inhibition of proliferation. Synergy scores were calculated and represented as a Bliss plot. FIG. 3F: SW1573 cells were treated with an increasing concentration of sotorasib (1-20 μM) with addition of carfilzomib (10 nM) for 72 hours to determine changes in protein expression and signaling by immunoblot.

FIGS. 4A-4E illustrate supporting data for FIGS. 3A-3F. FIG. 4A: H23, H358, and SW1573 cells were treated with ARS1620 IC50 concentration for 72 hours, and changes in protein expression and signaling were determined by immunoblot. FIG. 4B: H23 cells with ITGB4 overexpression (OE) were treated with an increasing concentration (0.3-10 μM) of sotorasib for 72 hours to determine percentage change in cell growth. FIG. 4C: H358, H23, and SW1573 cells were treated with half IC50 (IC50/2) and IC50 concentration of sotorasib for 72 hours. These same cells were made resistant to IC50 concentration of sotorasib (IC50 R), and changes in protein expression were determined by immunoblot. FIG. 4D: Highest synergy score for sotorasib and carfilzomib was calculated for SW1573 cells and represented as 3D Bliss plot. FIG. 4E: Percent of inhibition in SW1573 cells was determined with increasing concentration of sotorasib (1-64 μM) and fixed concentration of carfilzomib (9.5 nM) to determine which are antagonistic, additive, and synergistic.

FIGS. 5A-5J illustrate RNA sequencing reveals upregulation or downregulation of various genes involved in sotorasib resistance. FIG. 5A: RNA was isolated from H23 parental cells treated with IC50 dose (3 μM) of sotorasib for 3 days and H23 cells made resistant to 7.5 μM sotorasib over 7 weeks to perform RNA sequencing. Unregulated or downregulated genes were determined and are represented as a volcano plot. FIG. 5B: Number of genes upregulated or downregulated that were common and specific to each treatment condition represented as a Venn diagram. FIG. 5C: Clustering analysis of 712 genes that were downregulated or upregulated and common to the 3-day treatment group and the 7 weeks treatment group. FIG. 5D: List of 10 genes common to both treatment groups that were most upregulated and most downregulated. FIGS. 5E-5F: Gene set enrichment analysis was performed for both treatment groups, and data is represented from high to low enrichment score. FIG. 5G: Enrichment scores for KRAS signaling-up and KRAS signaling-down were negative and positive, respectively. FIG. 5H: qPCR data confirmed further upregulation of top 3 unregulated genes (CCL2, WNT2, CFTR) previously determined by RNA sequencing when H23 cells were treated with sotorasib for 7 weeks as compared to 3 days and untreated control cells. FIG. 5I: Using SYBR Green qPCR assay, RNA sequencing data was validated by confirming upregulation or downregulation of the other top 10 genes. FIG. 5J: H358, H23, and SW1573 cells treated with half IC50 dose and IC50 of sotorasib for 72 hours to determine changes in protein expression and signaling by immunoblot.

FIGS. 6A-6I illustrate WNT2/β-catenin pathway is involved in mediating resistance to sotorasib. FIG. 6A: Effect of WNT2 CRISPR knockout (KO) and addition of sotorasib IC50 on proliferation in H23 cells over the course of 72 hours. FIG. 6B: Effect of WNT2 CRISPR KO and addition of sotorasib IC50 on proliferation in SW1573 cells over the course of 72 hours. FIG. 6C: Effect of siITGB4 on WNT2 CRISPR KO and addition of sotorasib IC50 on proliferation in H23 cells over the course of 96 hours. FIG. 6D: Effect of ITGB4 and CTNNB1 single knockdowns and double knockdown with sotorasib 3 μM on H23 parental cell proliferation over 96 hours (line graph) and percent change in growth at 96 hours (bar graph). FIG. 6E: Effect of ITGB4 and CTNNB1 single knockdowns and double knockdown with sotorasib 3 μM on proliferation of H23 sotorasib (20 μM)-resistant cells over 96 hours (line graph) and percent change in growth at 96 hours (bar graph). FIG. 6F: Immunoblot confirmed knockdown of ITGB4 and CTNNB1 in H23 parental cells and H23 sotorasib (20 μM)-resistant cells. These cells were also treated with sotorasib 3 μM for 72 hours to identify changes in protein expression and signaling. FIG. 6G: Effect of ITGB4 and CTNNB1 single knockdowns and double knockdown with sotorasib 10 μM on SW1573 cell proliferation over 96 hours (line graph) and percent change in growth at 96 hours (bar graph). FIGS. 6H-6I: SW1573 cells were treated with 8 different concentrations of sotorasib and tegatrabetan in the form of a matrix to determine % inhibition of proliferation. Synergy scores were calculated and represented as a bliss plot. FIG. 6J: Highest synergy score for sotorasib and tegatrabetan was calculated for SW1573 cells and represented as 3D bliss plot.

FIGS. 7A-7C illustrate supporting data for FIGS. 6A-6J. FIG. 7A: Effect of WNT2 CRISPR knockout (KO) in and addition of increasing concentration of sotorasib treated for 72 hours on H23 cell signaling by immunoblot. FIG. 7B: Immunoblot confirmed knockdown of ITGB4 and CTNNB1 in SW1573 cells. These cells were also treated with sotorasib 8 μM for 72 hours to identify changes in protein expression and signaling. FIG. 7C: SW1573 cells were treated with an increasing concentration of sotorasib (8-16 μM) with addition of tegatrabetan (20 nM) for 72 hours to determine changes in protein expression and signaling by immunoblot.

FIGS. 8A-8H illustrates the effect of adagrasib on inhibiting growth of NSCLC cells. FIG. 8A: H358, H23, and SW1573 cells were treated with increasing concentration (0.6-10 μM) of adagrasib (MRTX) for 72 hours to determine effect on cell proliferation. FIG. 8B: H23, H358, and SW1573 cells were treated with adagrasib IC50 concentration for 72 hours, and changes in protein expression and signaling were determined by immunoblot. FIG. 8C: H358, H23, and SW1573 cell line-derived spheroids were treated with an increasing concentration (0.31-10 μM) of adagrasib, and images were taken with IncuCyte live cell imaging system on day 5. Red fluorescence indicates cell viability and green fluorescence indicates caspase-3/7 activity. FIG. 8D: Spheroid area and caspase-3/7 activity were determined over the course of 144 h. FIG. 8E: Effect of ITGB4 siRNA (siITGB4) and addition of adagrasib on cell proliferation over the course of 96 hours in H23 (top graph) and SW1573 cells (bottom graph). FIG. 8F: Effect of WNT2 CRISPR knockout (KO) and addition of adagrasib on cell proliferation over the course of 96 hours in H23 (top graph) and SW1573 cells (bottom graph). FIG. 8G: Effect of ITGB4/CTNNB1 double knockdown by siRNA and addition of adagrasib on cell proliferation over the course of 96 hours in H23 (top graph) and SW1573 cells (bottom graph). FIG. 8H: H23 parental cells and H23 sotorasib (20 μM)-resistant cells were treated with adagrasib (IC50/4, IC50/2, and IC50) to determine fold change in cell count over 96 hours.

FIGS. 9A-9C illustrate supporting data for FIGS. 8A-8H. FIG. 9A: H358, H23, and SW1573 cell line-derived spheroids were treated with an increasing concentration (0.31-10 μM) of adagrasib and spheroid red intensity was determined. FIG. 9B: Matrix of 6 different concentrations of adagrasib and 4 different concentrations of carfilzomib to determine percent of inhibition at various combinations of doses in SW1573 and H23 cells. FIG. 9C: Effect of increasing concentrations (0.5-2 μM) of adagrasib and combination with carfilzomib (20 nM) on cell proliferation over 96 hours in SW1573 cells.

FIGS. 10A-10F illustrate in vivo models confirm sensitivity of cells to combination of KRAS G12C inhibitors and carfilzomib. FIG. 10A: Brightfield images of zebrafish larvae with H23 xenotransplants labeled with green fluorescence dye. Images were taken after 3 days of sotorasib (12 μM), adagrasib (2 μM), and carfilzomib (1.6 μM) single treatments as well as drug combination treatments (sotorasib 6 μM+carfilzomib 0.4 μM, adagrasib 2 μM+carfilzomib 0.4 μM). FIG. 10B: Percent of tumor growth inhibition in zebrafish larvae after 3 days of drug treatment. FIGS. 10C-10D: Tumor weight (g) and area (mm2) of mice SW1573 xenografts after sotorasib (10 mg/kg), adagrasib (10 mg/kg), and carfilzomib (2 mg/kg) single treatments as well as drug combination treatments (sotorasib+carfilzomib, adagrasib+carfilzomib). FIG. 10E: Same increasing concentrations (1-5 μM) of sotorasib (AMG) and adagrasib (MRTX) exhibited different effects on cell proliferation over 20 h in H23 cells. FIG. 10F: Effect of increasing concentrations of sotorasib (1.25-20 μM) and adagrasib (0.6-10 μM) on cell cycle in SW1573 cells represented as FACS plots.

FIGS. 11A-11B illustrate supporting data for FIGS. 10C-10D. FIG. 11A: Photos of mice SW1573 xenografts from day 0 to day 56 (end of experiment) with sotorasib (10 mg/kg), adagrasib (10 mg/kg), and carfilzomib (2 mg/kg) single treatments as well as drug combination treatments (sotorasib+carfilzomib, adagrasib+carfilzomib). Photos of excised tumors on day 56. FIG. 11B: weight (g) of mice on day 0 and day 56 (end of experiment).

FIGS. 12A-12J illustrate KRAS G12C inhibitors can induce structural resistance and changes in phosphorylation signature. FIG. 12A: Molecular Dynamics simulations of KRAS G12C mutation in drug-free state and when bound to FIG. 12B sotorasib or FIG. 12C adagrasib. FIG. 12D: Measure of resistance to fluctuation of mutant molecule bound to drug compared to unbound molecule. FIG. 12E: Proteins with significant changes in phosphorylation when treated with sotorasib IC50 or FIG. 12F: adagrasib IC50 in SW1573 cells. FIG. 12G: Number of proteins with significant phosphorylation changes unique to sotorasib or adagrasib treatment or common to both treatments represented as a Venn diagram. FIG. 12H: Proteins significantly phosphorylated or dephosphorylated that are unique to sotorasib treatment. FIG. 12I: Proteins significantly phosphorylated or dephosphorylated that are unique to adagrasib treatment. FIG. 12J: Proteins significantly phosphorylated or dephosphorylated that are common with sotorasib or adagrasib treatment.

FIGS. 13A-13B illustrate supporting data for FIGS. 12A-12D. FIG. 13A: Contact probability maps depicting the formation of potential protein-drug interactions at an atomistic resolution limit for sotorasib (AMG) and FIG. 13B: adagrasib (MRTX).

FIGS. 14A-14D show the inhibitory effect of sotorasib or adagrasib on the LUAD cell lines H358, H23, and SW1573. FIG. 14A shows the percentage change of cell proliferation measured at 72 hours in the presence of increasing doses of sotorasib for the cell lines H358 (left panel), H23 (middle panel), and SW1573 (right panel). FIG. 14B shows the percentage change of cell proliferation measured at 72 hour in the presence of increasing doses of adagrasib for the cell lines H358 (left panel), H23 (middle panel), and SW1573 (right panel). FIGS. 14C-14D show the cell viability and dose response curve for the three cell lines H358, H23, and SW1573 to determine the IC50 dose for sotorasib (FIG. 14C) and adagrasib (FIG. 14D). In FIGS. 14C-14D, with reference to a concentration of 2 μM, the upper line is SW1573, the middle line is H23, and the bottom line is H358.

FIGS. 15A-15D show the results of 3D spheroid assay of the effect of sotorasib and adagrasib in in-vivo mimic conditions. FIG. 15A provides images of the cell line derived spheroids collected at 72 hours after sotorasib treatment. The green fluorescence represents caspase 3/7 activity which is indication of apoptosis. More particularly, apoptosis is evident in the H358 cell line at doses from 0.15 μM to 10 μM; and in the H23 cell line at doses from 0.31 μM to 10 μM, although there is evidence of apoptosis at the 0.15 μM as well. FIG. 15B is a bar graph quantifying caspase activity following treatment with sotorasib. The bars from left to right at each concentration are caspase activity for H358, H23, SW1573 with increasing concentrations of sotorasib. The highest caspase activity was for H358, followed by H23, with SW1573 showing the lowest caspase activity. FIG. 15C provides images of the cell line derived spheroids collected at 72 hours after adagrasib treatment. The green fluorescence represents caspase 3/7 activity which is indication of apoptosis. More particularly, apoptosis is evident in the H358 cell line at doses from 0.6 μM to 10 μM, although there is evidence of apoptosis at 0.3 μM as well; and in the H23 cell line at doses from 0.6 μM to 10 μM. FIG. 15D is a bar graph quantifying caspase activity following treatment with adagrasib. The bars from left to right at each concentration are caspase activity for H23, H358, SW1573 with increasing concentrations of adagrasib. The highest caspase activity was for H23, followed by H358, with SW1573 showing the lowest caspase activity; however, at concentration of 10 μM, the caspase activity for H358 was only slightly greater than the caspase activity for SW1573.

FIGS. 16A-16D show changes in the signaling response to KRAS inhibition in various cell lines after 72 hr of treatment with the IC50 dose of the inhibitors sotorasib and adagrasib.

FIGS. 17A-17G show the various genes that are upregulated or downregulated in response to treatment with sotorasib. FIG. 17A shows a set of common genes which changed their expression in the H23 cell line with continuous exposure to sotorasib. The blue color indicates the genes which have low expression and red color indicate genes with higher expression. FIG. 17B shows the top genes which showed more than 5-fold change within 3 days of sotorasib exposure. FIG. 17C shows the top genes which showed more than 5-fold change on continuous exposure to sotorasib for 7 weeks. FIGS. 17D-17F provide the validation of the RNA seq results for the genes CFTR, WNT2, and CCL2 by qPCR. FIG. 17G shows that treating H358 and H23 cells with sublethal dose of sotorasib, increased the expression of Wnt2, as measured by western blotting. In FIGS. 17D-17F, the upper panel is Exp2, and the lower panel is Exp1.

FIGS. 18A-18F show the results of increasing dose of sotorasib or adagrasib on the H23 cell line proliferation at the earlier time points from the time of drug addition to hour 20. In FIGS. 18A-185, with reference to 20 hours, the upper line is untreated, the middle line is sotorasib, and the lower line is adagrasib.

FIGS. 19A-19E show the results of carfilzomib in sensitizing sotorasib resistant H23, H358 and SW1573 cell lines. FIGS. 19A-19B show the drug response and the IC50 dose of Carfilzomib for the sotorasib tolerant cell lines H23 and SW1573. FIGS. 19C-19D show the inhibition matrix and the synergy plot generated against the SW1573 cells by combining various doses of sotorasib and carfilzomib. FIG. 19E shows the changes in signaling due to the combination of sotorasib and carfilzomib in the SW1573 cell line.

FIGS. 20A-20C show the results of an assay of the individual and combined effect of carfilzomib and adagrasib on cell proliferation. FIG. 20A provides results for untreated cells (top line at 48 hours); cells treated with 0.5 μM adagrasib (immediately below top line at 48 hours); cells treated with 20 nM carfilzomib (immediately above bottom line at 48 hours); and cells treated with the combination of 0.5 μM adagrasib and 20 nM carfilzomib (bottom line at 48 hours). FIG. 20A shows that 0.5 μM adagrasib could not inhibit cell proliferation whereas the combination of adagrasib and carfilzomib has an additive effect. FIG. 20B provides results for untreated cells (top line at 48 hours); cells treated with 1 μM adagrasib (immediately above bottom line at 48 hours); cells treated with 20 nM carfilzomib (immediately below top line at 48 hours); and cells treated with the combination of 1 μM adagrasib and 20 nM carfilzomib (bottom line at 48 hours). FIG. 20B shows that adagrasib effectively inhibited cell proliferation by 25% and the combination of adagrasib and carfilzomib inhibited proliferation by 53%, which was a synergistic result. FIG. 20C provides results for untreated cells (top line at 48 hours); cells treated with 2 μM adagrasib (immediately above bottom line at 48 hours); cells treated with 20 nM carfilzomib (immediately below top line at 48 hours); and cells treated with the combination of 2 μM adagrasib and 20 nM carfilzomib (bottom line at 48 hours). FIG. 20C shows that adagrasib has a strong inhibitory effect on cell proliferation.

FIGS. 21A-21D show the effects of ITGB4 knockdown on adagrasib drug response. FIG. 21A shows that 0.5 μM adagrasib inhibited cell proliferation by 38% and ITGB4 knockdown inhibited cell proliferation by 34%, but the combination of knockdown and adagrasib reduce proliferation by 50%, indicating ITGB4 knockdown increase drug sensitivity. At the 72 hour point, the top line is the siControl, the line immediately below the top line is 0.5 adagrasib and siControl, the line immediately above the bottom line is siITGB4, and the bottom line is 0.5 μM adagrasib and SiITGB4. FIG. 21B shows that 1 μM adagrasib effectively inhibited cell proliferation by 50% and the combination further inhibited cell proliferation. At the 72 hour point, the top line is the siControl, the line immediately below the top line is siITGB4, the line immediately above the bottom line is 1 μM adagrasib and siControl, and the bottom line is 1 μM adagrasib and SiITGB4. FIG. 21C shows that 2 μM adagrasib has a strong inhibitory effect on cell proliferation. At the 72 hour point, the top line is the siControl, the line immediately below the top line is siITGB4, the line immediately above the bottom line is 2 μM adagrasib and siControl, and the bottom line is 2 μM adagrasib and SiITGB4. In FIG. 21D, immunoblotting shows ITGB4 knockdown, where the addition of adagrasib effectively inhibited the activated form of β-catenin.

FIGS. 22A-22D show the effect of sotorasib and adagrasib drug response in the colorectal G12C mutant cell lines. At 72 hours, the top line represents the control of cell proliferation without any drug. FIG. 22A shows that the heterozygous mutant SW837 cell line is highly sensitive to the lowest dose (0.16 μM) of sotorasib, and that increasing the dose of sotorasib does not increase cell inhibition. FIG. 22B shows that the 10 μM dose of sotorasib inhibited cell proliferation by 40%, indicating that homozygous mutants are more tolerant to sotorasib. FIG. 22C shows that a minimal drug concentration of 0.16 μM of adagrasib had a strong inhibitory effect on the homozygous cell line and doses of 5 μM or 10 μM (bottom two lines at 72 hours) adagrasib induced cell death within 72 hours. FIG. 22D indicates that SW1463 showed a dose-dependent response to adagrasib, where, a dose of 2.5 μM adagrasib is cytostatic (third line from bottom at 72 hours), and a dose of 10 μM adagrasib was toxic to the cells (bottom line at 72 hours).

FIG. 23 shows the results at 24 hours, 48 hours, and 72 hours post-injection of vehicle (left panel) or the combination of 6 μM sotorasib and 400 nM carfilzomib (right panel). The results demonstrate that combination of 6 μM sotorasib and 400 nM carfilzomib effectively inhibited the proliferation of SW1573 cells.

FIG. 24 shows some aspects of the RAS signaling pathway, where GDP refers to RAS bound to guanosine diphosphate (the off state), and GTP refers to RAS bound to guanosine triphosphate (the on state).

### DETAILED DESCRIPTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “integrin β4 protein” or “ITGB4 protein” or “ITGB4” as used herein includes any of the recombinant or naturally-occurring forms of ITGB4 or variants or homologs thereof that maintain ITGB4 activity (e.g. within at least 50%, 80%, 90%, 95%, 96%, 97%, 98%, 99% or 100% activity compared to ITGB4). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ITGB4 protein. In aspects, the ITGB4 protein is substantially identical to the protein identified by the UniProt reference number P16144 or a variant or homolog having substantial identity thereto.

The term “paxillin protein” or “PXN protein” or “PXN” as used herein includes any of the recombinant or naturally-occurring forms of PXN or variants or homologs thereof that maintain PXN activity (e.g. within at least 50%, 80%, 90%, 95%, 96%, 97%, 98%, 99% or 100% activity compared to PXN). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PXN protein. In aspects, the PXN protein is substantially identical to the protein identified by the UniProt reference number P49023 or a variant or homolog having substantial identity thereto.

The term “KRAS” or “KRAS protein” or “KRas protein” or “KRas” or “GTPase KRas” as used herein includes any of the recombinant or naturally-occurring forms of KRas or variants or homologs thereof that maintain KRAS activity (e.g. within at least 50%, 80%, 90%, 95%, 96%, 97%, 98%, 99% or 100% activity compared to KRAS). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KRAS protein. The KRAS protein can be isoform 2A or isoform 2B. In aspects, the KRAS protein is substantially identical to the protein identified by the UniProt reference number P01116 or a variant or homolog having substantial identity thereto.

The term “Wnt” or “Wnt protein” as used herein includes any of the recombinant or naturally-occurring forms of Wnt or variants or homologs thereof that maintain Wnt activity (e.g. within at least 50%, 80%, 90%, 95%, 96%, 97%, 98%, 99% or 100% activity compared to Wnt). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Wnt protein. In aspects, the Wnt protein is substantially identical to the protein identified by the UniProt reference number A0A087WXR9 or a variant or homolog having substantial identity thereto.

The term “β-catenin” or “β-catenin protein” as used herein includes any of the recombinant or naturally-occurring forms of β-catenin or variants or homologs thereof that maintain β-catenin activity (e.g. within at least 50%, 80%, 90%, 95%, 96%, 97%, 98%, 99% or 100% activity compared to (3-catenin). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring β-catenin protein. In aspects, the β-catenin protein is substantially identical to the protein identified by the UniProt reference number P35222 or a variant or homolog having substantial identity thereto.

The term “KRAS mutation” refers to a mutation in one or more amino acids in the KRAS protein. In aspects, KRAS mutation refers to a mutation in one or more codons in the KRAS gene. In aspects, the KRAS mutation refers to a mutation at codon 12 in the KRAS gene a mutation at codon 13 in the KRAS gene, a mutation at codon 61 in the KRAS gene, or a combination of two or more thereof. In aspects, the KRAS mutation refers to a G12 mutation in the KRAS protein, a KRAS G13 mutation in the KRAS protein, a KRAS H61 mutation in the KRAS protein, or a combination of two or more thereof. In aspects, the KRAS mutation refers to a G12C mutation in the KRAS protein. In aspects, the KRAS mutation refers to a G12V mutation in the KRAS protein. In aspects, the KRAS mutation refers to a G12A mutation in the KRAS protein. In aspects, the KRAS mutation refers to a G12D mutation in the KRAS protein.

The term “heterozygous” refers to a mutation of only one allele. For example, a heterozygous codon 12 KRAS mutation refers to a mutation on codon 12 that occurs on one allele.

The term “homozygous” refers to an identical mutation of both the parental and maternal alleles. For example, a homozygous codon 12 KRAS mutation refers to a mutation on codon 12 that occurs on both alleles.

The term “inhibitor,” “inhibition,” “inhibit,” “inhibiting” and the like in reference to a protein-inhibitor interaction means negatively affecting (e.g., decreasing) the activity or function of the protein (e.g., decreasing the activity of ITGB4, PXN, KRAS, Wnt, (3-catenin) relative to the activity or function of the protein in the absence of the inhibitor. In aspects, inhibition refers to reduction of a disease or symptoms of disease (e.g., cancer). Thus, inhibition includes, at least in part, partially or totally blocking stimulation, decreasing, preventing, or delaying activation, or inactivating, desensitizing, or down-regulating signal transduction or enzymatic activity or the amount of a protein (e.g., a ITGB4 protein, a PXN protein, a KRAS protein, a Wnt protein, a β-catenin protein). Similarly an “inhibitor” is a compound or protein that inhibits a receptor or a protein, e.g., by binding, partially or totally blocking, decreasing, preventing, delaying, inactivating, desensitizing, or down-regulating activity (e.g., ITGB4 protein activity, PXN protein activity, KRAS protein activity, Wnt protein activity, (3-catenin protein activity).

The term “ITGB4/PXN inhibitor” refers to therapeutic agents that perturb the interaction between integrin (ITGB4) and paxillin (PXN), e.g., by inhibiting the expression of ITGB4, by inhibiting the expression of PXN, by inhibiting the expression of both ITGB4 and PXN. In aspects, the ITGB4/PXN inhibitor inhibits the expression of ITGB4. In aspects, the ITGB4/PXN inhibitor inhibits the expression of PXN. In aspects, the ITGB4/PXN inhibitor inhibits the expression of ITGB4 and PXN. In aspects, the ITGB4/PXN inhibitor inhibits an interaction between ITGB4 and PXN. In aspects, the ITGB4/PXN inhibitor is an anti-ITGB4 antibody, an RNA (e.g., siRNA) that inhibits the expression of ITGB4, a small molecule that inhibits the expression of ITGB4, an anti-PXN antibody, an RNA (e.g., siRNA) that inhibits the expression of PXN, or a small molecule that inhibits the expression of PXN. Exemplary ITGB4/PXN inhibitors include, without limitation, carfilzomib, tozasertib, dasatinib, afatinib, dacomitinib, poziotinib, pacritinib, ixazomib, osimertinib, CUDC-101 (i.e., 7-((4-((3-ethynylphenyl)amino)-7-methoxyquinazolin-6-yl)oxy)-N-hydroxyheptanamide), and cerdulatinib. Carfilzomib is commercially available as KYPROLIS® from Amgen, Inc. Carfilzomib can alternatively be referred to as CFZ.

In embodiments, the “ITGB4/PXN inhibitor” is a “ITGB4/PXN pathway inhibitor.” In aspects, a ITGB4/PXN pathway inhibitor refers to a therapeutic agent (e.g., compound, antibody, RNA) capable of detectably lowering expression of or activity level of the ITGB4 and/or PXN signaling pathway compared to a control. The inhibited expression or activity of the ITGB4 and/or PXN signaling pathway can be 10%, 20%, 30%, 40%, 50%, 60%, 70%, 80%, 90% or less than that in a control. In aspects, the inhibition is 1.5-fold, 2-fold, 3-fold, 4-fold, 5-fold, 10-fold, or more in comparison to a control. A ITGB4/PXN pathway inhibitor is capable of inhibiting the ITGB4 and/or PXN pathway signaling pathway e.g., by binding, partially, or totally blocking stimulation of the ITGB4 and/or PXN signaling pathway; decrease, prevent, or delay activation of the ITGB4 and/or PXN signaling pathway; or inactivate, desensitize, or down-regulate signal transduction, gene expression, or enzymatic activity of the ITGB4 and/or PXN signaling pathway.

The term “Wnt/β-catenin pathway inhibitor” refers to a therapeutic agent that is capable of detectably lowering expression of or activity level of the Wnt and/or β-catenin signaling pathway compared to a control. The inhibited expression or activity of the Wnt and/or β-catenin signaling pathway can be 10%, 20%, 30%, 40%, 50%, 60%, 70%, 80%, 90% or less than that in a control. In aspects, the inhibition is 1.5-fold, 2-fold, 3-fold, 4-fold, 5-fold, 10-fold, or more in comparison to a control. A Wnt/β-catenin pathway inhibitor is capable of inhibiting the Wnt and/or β-catenin pathway signaling pathway e.g., by binding, partially, or totally blocking stimulation of the Wnt and/or β-catenin signaling pathway; decrease, prevent, or delay activation of the Wnt and/or β-catenin signaling pathway; or inactivate, desensitize, or down-regulate signal transduction, gene expression, or enzymatic activity of the Wnt and/or β-catenin signaling pathway. In aspects, the Wnt/β-catenin pathway inhibitor is an antibody, an RNA (e.g., siRNA), or a small molecule. Exemplary Wnt/β-catenin pathway inhibitor include, without limitation, tegatrabetan (BC2059), LF3 (N-[4-(aminosulfonyl)phenyl]-4-(3-phenyl-2-propen-1-yl)-1-piperazinecarbothioamide)), KYA1797K ((5Z)-5-[[5-(4-nitrophenyl)-2-furanyl]-methylene]-4-oxo-2-thioxo-3-thiazolidinepropanoic acid)), KY1220 ((5Z)-5-[[1-(4-nitrophenyl)pyrrol-2-yl]methylidene]-2-sulfanylideneimidazolidin-4-one), iCRT3 (2-[[[2-(4-ethylphenyl)-5-methyl-4-oxazolyl]methyl]thio]-N-(2-phenylethyl)acetamide), iCRT5 (4-[5-(3,4-dimethoxy-benzylidene)-4-oxo-2-thioxo-thiazolidin-3-yl]-butyric acid), iCRT14 (5-[[2,5-dimethyl-1-(3-pyridinyl)-1H-pyrrol-3-yl]methylene]-3-phenyl-2,4-thiazolidinedioner), ZINC02092166 ((Z)-4-amino-N′-(9H-indeno[1,2-b][1,2,5]oxadiazolo[3,4-e]pyrazin-9-ylidene)-1,2,5-oxadiazole-3-carbohydrazide), NLS-StAx-h, foscenvivint (PRI-724), tabituximab barzuxetan, vantictumab (OMP-18R5), ipafricept (OMP-54F28), Fz7-21, salinomycin, FJP ((2E,6Z)-3,7,11-trimethyldodeca-2,6,10-trien-1-yl dihydrogen phosphate), 3289-8625 (BML-286) (2-((3-(2-phenylacetyl)amino)benzoyl)amino)benzoic acid), XAV939 (3,5,7,8-tetrahydro-2-[4-(trifluoromethyl)phenyl]-4H-thiopyrano[4,3-d]pyrimidin-4-one), JW74 (methoxyphenyl)-5-[[[3-(4-methylphenyl)-1,2,4-oxadiazol-5-yl]methyl]thio]-4H-1,2,4-triazol-3-yl]-pyridine), JW55 (N-[4-[[[[tetrahydro-4-(4-methoxyphenyl)-2H-pyran-4-yl]methyl]amino]carbonyl]-phenyl]-2-furancarboxamide), NVP-TNKS656 (N-(cyclopropylmethyl)-4-(4-methoxybenzoyl)-N-[(3,5,7,8-tetrahydro-4-oxo-4H-pyrano[4,3-d]pyrimidin-2-yl)methyl]-1-piperidineacetamide), LZZ-02 ((E)-3-((4-nitrophenyl)amino)-1-phenylprop-2-en-1-one), SSTC3 (4-(N-methyl-N-(4-(trifluoromethyl)phenyl)sulfamoyl)-N-(4-(pyridin-2-yl)thiazol-2-yl)benzamide), WNT974 (2-(2′,3-dimethyl-[2,4′-bipyridin]-5-yl)-N-(5-(pyrazin-2-yl)pyridin-2-yl)acetamide), ETC-159 (1,2,3,6-tetrahydro-1,3-dimethyl-2,6-dioxo-N-(6-phenyl-3-pyridazinyl)-7H-purine-7-acetamide), CGX1321 (N-(3-fluoro-4-(2-fluoropyridin-4-yl)benzyl)-6-(2-Methylpyridin-4-yl)-2,7-naphthyridin-1-amine), GNF-6231 (N-(5-(4-acetylpiperazin-1-yl)pyridin-2-yl)-2-(2′-fluoro-3-methyl-[2,4′-bipyridin]-5-yl)acetamide), ICG001 ((6S,9aS)-hexahydro-6-[(4-hydroxyphenyl)-methyl]-8-(1-naphthalenylmethyl)-4,7-dioxo-N-(phenylmethyl)-2H-pyrazino[1,2-a]pyrimidine-1(6H)-carboxamide), isoquercitrin, FJ9 (2-(1-hydroxypentyl)-6-methyl-3-phenethyl-1H-indole-5-carboxylic acid), IWP-2 (N-(6-methyl-2-benzothiazolyl)-2-[(3,4,6,7-tetrahydro-4-oxo-3-phenylthieno[3,2-d]pyrimidin-2-yl)thio]-acetamide), IWP-4 (N-(6-methyl-2-benzothiazolyl)-2-[[3,4,6,7-tetrahydro-3-(2-methoxyphenyl)-4-oxothieno[3,2-d]pyrimidin-2-yl]thio]-acetamide), calphostin C (PFK115-584 or (1R)-2-[12-[(2R)-2-(Benzoyloxy)propyl]-3,10-dihydro-4,9-dihydroxy-2,6,7,11-tetramethoxy-3,10-dioxo-1-perylenyl]-1-methylethylcarbonic acid 4-hydroxyphenyl ester), DKN-01, CWP291 (CWP232291), GNE-781 (3-(7-(Difluoromethyl)-6-(1-methyl-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide), Wnt-059 (4-(2-Methyl-4-pyridinyl)-N-[4-(3-pyridinyl)phenyl]benzeneacetamide), endo-IWR-1 ([(3aR*,4S*,7R*,7aS)-1,3,3a,4,7,7a-hexahydro-1,3-dioxo-4,7-methano-2H-isoindol-2-yl]-N-8-quinolinylbenzamide), niclosamide, ONC201 (2,4,6,7,8,9-hexahydro-4-[(2-methylphenyl)methyl]-7-(phenylmethyl)-imidazo[1,2-a]pyrido[3,4-e]pyrimidin-5(1H)-one), TFP (tri(2-furyl)phosphine), actinomycin D/telmisartan combination, chelerythrine, IC-2, JIB-04 (5-chloro-2-[(E)-2-[phenyl(pyridin-2-yl)methylidene]hydrazin-1-yl]pyridine), FH535 (2,5-dichloro-N-(2-methyl-4-nitrophenyl)-benzenesulfonamide), docataxel/sulforaphane combination, pyrvinium pamoate, and SKL2001 (5-(2-furanyl)-N-[3-(1H-imidazol-1-yl)propyl]-3-isoxazolecarboxamide).

The term “KRAS inhibitor” refers to therapeutic agents that inhibit KRAS. In aspects, the KRAS inhibitor is a KRAS codon 12 inhibitor, KRAS codon 13 inhibitor, a KRAS codon 61 inhibitor, or a combination of two or more thereof. In aspects, the KRAS inhibitor is a KRAS G12 inhibitor, a KRAS G13 inhibitor, a KRAS H61 inhibitor, or a combination of two or more thereof. In aspects, the KRAS inhibitor is a KRAS G12C inhibitor, a KRAS G12A inhibitor, a KRAS G12V inhibitor, a KRAS G12D inhibitor, or a combination of tow or more thereof. Exemplary KRAS inhibitors include, without limitation, sotorasib, adagrasib, MRTX1133, ARS1620, BI-1701963, BI-3406 (i.e., N-((R)-1-(3-amino-5-(trifluoromethyl)phenyl)ethyl)-7-methoxy-2-methyl-6-(((S)-tetrahydrofuran-3-yl)oxy)quinazolin-4-amine), compound 0375-0604, LY3499446, LY3537982, JNJ-74699157, TNO155 (i.e., (3S,4S)-8-(6-amino-5-((2-amino-3-chloroyridin-4-yl)thio)pyrazin-2-yl)-3-methyl)2-oxa-8-azaspiro[4.5]decan-4-amine), and ARS1620 (i.e., (S)-1-(4-(6-chloro-8-fluoro-7-(2-fluoro-6-hydroxyphenyl)quinazolin-4-yl)piperazin-1-yl)prop-2-en-1-one). Sotorasib is alternatively referred to as AMG510. Adagrasib is alternatively referred to as MRTX849.

In aspects, the “KRAS inhibitor” is a “KRAS pathway inhibitor.” In aspects, a KRAS pathway inhibitor refers to a therapeutic agent (e.g., compound, antibody, RNA) capable of detectably lowering expression of or activity level of the KRAS signaling pathway compared to a control. The inhibited expression or activity of the KRAS signaling pathway can be 10%, 20%, 30%, 40%, 50%, 60%, 70%, 80%, 90% or less than that in a control. In aspects, the inhibition is 1.5-fold, 2-fold, 3-fold, 4-fold, 5-fold, 10-fold, or more in comparison to a control. A KRAS pathway inhibitor is capable of inhibiting the KRAS signaling pathway, e.g., by binding, partially or totally blocking stimulation of the KRAS signaling pathway; decrease, prevent, or delay activation of the KRAS signaling pathway; or inactivate, desensitize, or down-regulate signal transduction, gene expression, or enzymatic activity of the KRAS signaling pathway.

“Previously treated with KRAS inhibitor” refers to a subject that had been treated with a KRAS inhibitor in the past or treated with a KRAS inhibitor prior to treatment with an ITGB4/PXN inhibitor. In aspects, the subject previously treated with a KRAS inhibitor was previously treated with sotorasib, adagrasib, BI-3406, or BI 1701963. In aspects, the subject previously treated with a KRAS inhibitor was previously treated sotorasib, adagrasib, MRTX1133, BI-1701963, BI-3406, compound 0375-0604, LY3499446, LY3537982, JNJ-74699157, TNO155, or ARS1620. In aspects, the subject previously treated with a KRAS inhibitor was previously treated sotorasib, adagrasib, BI-1701963, BI-3406, compound 0375-0604, LY3499446, LY3537982, JNJ-74699157, TNO155, or ARS1620. In aspects, the subject previously treated with a KRAS inhibitor is an “anti-KRAS inhibitor refractory subject” or a “refractory subject.” In aspects, the subject previously treated with a KRAS inhibitor is an “anti-KRAS inhibitor resistant subject” or a “resistant subject.” In aspects, the subject was responsive to prior treatment with the KRAS inhibitor, prior to become refractory or resistant.

“KRAS inhibitor resistant subject” or “resistant subject” or “subject resistant to treatment with a KRAS inhibitor” refer to cancer patients who are initially responsive to treatment with a KRAS inhibitor, but then became resistant to the KRAS inhibitor over time. “Resistant subjects” have been generally been treated with a KRAS inhibitor for more than one, two, or three months. The “resistant subject” initially showed some benefits from treatment with the KRAS inhibitor, where the benefits could have been: (i) a cancerous tumor that did not grow in size or volume; (ii) a cancerous tumor that decreased in size or volume; (iii) a cancerous tumor that did not metastasize; or (iv) a combination of two or more of the foregoing. After initially showing a benefit to treatment, the “resistant subject” then became unresponsive to treatment with the KRAS inhibitor. Where the cancer patient is unresponsive to treatment with the KRAS inhibitor, the patient shows less than 20% reduction in tumor size or volume after administration of the KRAS inhibitor relative to a control. In aspects, a resistant subject shows no reduction in tumor size or volume after administration of the KRAS inhibitor relative to a control. In aspects, a resistant subject shows an increase in tumor size or volume after administration of the KRAS inhibitor relative to a control. A subject can be identified as a “resistant subject” by measuring the expression level of ITGB4, PXN, or both in a biological sample obtained from the subject, as described herein.

“Subject naïve to treatment with a KRAS inhibitor” refers to a subject that had not previously been treated with a KRAS inhibitor.

“Subject responsive to prior KRAS inhibitor treatment” or refers to a subject that had been treated with a KRAS inhibitor prior to the initiation of treatment with the combination of an ITGB4/PXN inhibitor and a KRAS inhibitor, wherein the subject had been responsive to treatment with the KRAS inhibitor, but then became a “resistant subject.” “Responsive” indicates that: (i) the cancerous tumor had not grown in size or volume over time; (iii) the cancerous tumor had decreased in size or volume over time; (iv) the cancerous tumor had not metastasized; or (v) a combination of two or more of the foregoing. In aspects, a “subject responsive to prior KRAS inhibitor treatment” had shown a decrease (i.e., reduction) in tumor size or volume during/after treatment compared to baseline or a control. In aspects, a subject responsive to prior KRAS inhibitor treatment had shown at least a 20% reduction in tumor size or volume during/after treatment compared to baseline or a control.

“Biological sample” refers to any biological sample taken from a subject. Biological samples include blood, plasma, serum, tumors, tissue, cells, and the like. In aspects, the biological sample is a blood sample. In aspects, the biological sample is a peripheral blood sample. In aspects, the biological sample is a tumor sample. In aspects, the biological sample is a primary tumor sample. In aspects, the biological sample is a metastatic tumor sample. In aspects, the biological sample is a resected tumor sample. In aspects, the biological sample is a tumor biopsy sample. In aspects, the biological sample is a resected tumor sample from a primary tumor. In aspects, the biological sample is a resected tumor sample from a metastisic tumor. In aspects, the biological sample is a tumor biopsy sample from a primary tumor. In aspects, the biological sample is a tumor biopsy sample from a metastisic tumor. Biological samples can be taken from a subject by methods known in the art, and can be analyzed by methods known in the art.

A “control” sample or value refers to a sample that serves as a reference, usually a known reference, for comparison to a test sample. For example, a test sample can be taken from a patient suspected of having a given disease (cancer) and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In aspects, a control is a negative control. In aspects, such as embodiments relating to detecting the level of expression or infiltration, a control comprises the average amount of expression (e.g., protein or mRNA) of infiltration (e.g., number or percentage of cells in a population of cells) in a population of subjects (e.g., with cancer) or in a healthy or general population. In aspects, the control comprises an average amount (e.g. percentage or number of infiltrating cells or amount of expression) in a population in which the number of subjects (n) is 5 or more, 10 or more, 25 of more, 50 or more, 100 or more, 1000 or more, 5000 or more, or 10000 or more. In aspects, the control is a standard control. In aspects, the control is a population of cancer subjects who are KRAS resistant or KRAS refractory. In aspects, the control is a tumor sample from a population of cancer subjects who are KRAS resistant or KRAS refractory. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein “treating cancer” means preventing an increase in size or volume of the cancer tumor. In aspects, treating a cancer tumor includes decreasing the size of volume of a cancer tumor. In aspects, treating a cancer tumor includes eliminating the cancer tumor altogether. In aspects, a cancer tumor is eliminated when it is not detectable by an imaging test such as magnetic resonance imaging (MRI), a positron emission tomography (PET) scan, X-ray computed tomography (CT), ultrasound, or single-photon emission computed tomography (SPECT). In aspects, treating a cancer tumor further comprises reducing or preventing metastasis of the cancer tumor.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer (e.g., squamous cell carcinoma of the head and neck), kidney cancer (e.g., renal cell carcinoma), myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiform, glioma, melanoma, prostate cancer, castration-resistant prostate cancer, metastatic castration resistant prostate cancer, breast cancer, triple negative breast cancer, glioblastoma, ovarian cancer, lung cancer, squamous cell carcinoma (e.g., head, neck, or esophagus), colorectal cancer (e.g., microsatellite instable colorectal cancer), leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or medulloblastoma, Hodgkin's disease, non-Hodgkin's lymphoma, multiple myeloma, neuroblastoma, glioma, glioblastoma multiform, ovarian cancer, primary thrombocytosis, rhabdomyosarcoma, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, papillary thyroid cancer, hepatocellular carcinoma, Paget's disease of the nipple, phyllodes tumors, lobular carcinoma, ductal carcinoma, cancer of the pancreatic stellate cells, cancer of the hepatic stellate cells, or prostate cancer.

The term “solid tumor” refers to a mass of cancer cells, generally devoid of cysts or liquid areas. The mass of cells can include cancer cells, cancer stem cells, connective-tissue cells, immune cells, and the like. Solid tumors include carcinoma, sarcoma, and melanoma. The term “carcinoma” refers to a malignant new growth made up of epithelial cells tending to infiltrate the surrounding tissues and give rise to metastases. The term “sarcoma” generally refers to a tumor which is made up of a substance like the embryonic connective tissue and is generally composed of closely packed cells embedded in a fibrillar or homogeneous substance. The term “melanoma” refers to a tumor arising from the melanocytic system of the skin and other organs.

As used herein, the terms “metastasis,” “metastatic,” “metastatic tumor,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

A “patient” or “subject” include both humans and other animals, particularly mammals. Thus, the methods are applicable to both human therapy and veterinary applications. In aspects, the patient is a mammal. In aspects, the patient is a companion animal, such as a dog or a cat. In aspects, the patient is human.

Methods

In embodiments, the disclosure provides methods of treating cancer in a subject by administering to the subject an effective amount of an ITGB4/PXN inhibitor and an effective amount of a KRAS inhibitor. In aspects, the cancer has a KRAS mutation. In aspects, the subject is resistant to treatment with a KRAS inhibitor. In aspects, the methods further comprising administering to the subject an effective amount of a Wnt/β-catenin pathway inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods of treating cancer in a subject by administering to the subject an effective amount of a Wnt/β-catenin pathway inhibitor and an effective amount of a KRAS inhibitor. In aspects, the cancer has a KRAS mutation. In aspects, the subject is resistant to treatment with a KRAS inhibitor.

In embodiments, the disclosure provides methods to select a subject with cancer for treatment with an ITGB4/PXN inhibitor and a KRAS inhibitor, the method comprising measuring a KRAS mutation in a biological sample obtained from the subject; wherein if the KRAS mutation is present in the biological sample, the subject is selected for treatment with the ITGB4/PXN inhibitor and the KRAS inhibitor. In aspects, the method further comprises administering to the subject an effective amount of an ITGB4/PXN inhibitor and an effective amount of a KRAS inhibitor. In aspects, the method further comprises administering to the subject an effective amount of an ITGB4/PXN inhibitor, an effective amount of a Wnt/β-catenin pathway inhibitor, and an effective amount of a KRAS inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods to select a subject with cancer for treatment with a Wnt/β-catenin pathway inhibitor and a KRAS inhibitor, the method comprising measuring a KRAS mutation in a biological sample obtained from the subject; wherein if the KRAS mutation is present in the biological sample, the subject is selected for treatment with the Wnt/β-catenin pathway inhibitor and the KRAS inhibitor. In aspects, the method further comprises administering to the subject an effective amount of a Wnt/β-catenin pathway inhibitor and an effective amount of a KRAS inhibitor.

In embodiments, the disclosure provides methods to identify a subject with cancer who would be responsive to treatment with a ITGB4/PXN inhibitor and a KRAS inhibitor, the method comprising measuring a KRAS mutation in a biological sample obtained from the subject; wherein if the KRAS mutation is present in the biological sample, the subject is identified as being responsive to treatment with the ITGB4/PXN inhibitor and the KRAS inhibitor. In aspects, the method further comprises administering to the subject an effective amount of an ITGB4/PXN inhibitor and an effective amount of a KRAS inhibitor. In aspects, the method further comprises administering to the subject an effective amount of an ITGB4/PXN inhibitor, an effective amount of a Wnt/β-catenin pathway inhibitor, and an effective amount of a KRAS inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods to identify a subject with cancer who would be responsive to treatment with a Wnt/β-catenin pathway inhibitor and a KRAS inhibitor, the method comprising measuring a KRAS mutation in a biological sample obtained from the subject; wherein if the KRAS mutation is present in the biological sample, the subject is identified as being responsive to treatment with the Wnt/β-catenin pathway inhibitor and the KRAS inhibitor. In aspects, the method further comprises administering to the subject an effective amount of a Wnt/β-catenin pathway inhibitor and an effective amount of a KRAS inhibitor.

In embodiments, the disclosure provides methods of detecting resistance to cancer treatment with a KRAS inhibitor in a subject in need thereof, the method comprising: (i) measuring the expression level of ITGB4, PXN, or both in a biological sample obtained from the subject; (ii) comparing the expression level of ITGB4, PXN, or both in the biological sample obtained from the subject to the expression level of ITGB4, PXN, or both in a control; wherein an elevated expression level of ITGB4, PXN, or both indicates the subject is resistant to treatment with the KRAS inhibitor. In aspects, the method comprises measuring the expression level of ITGB4 in the biological sample and comparing the expression level of ITGB4 obtained from the subject to the control. In aspects, the method comprises measuring the expression level of PXN in the biological sample and comparing the expression level of PXN obtained from the subject to the control. In aspects, the method comprises measuring the expression level of ITGB4 and PXN in the biological sample and comparing the expression level of ITGB4 and PXN obtained from the subject to the control. In aspects, the method further comprises administering to the subject an effective amount of an ITGB4/PXN inhibitor and a KRAS inhibitor when the subject has an elevated expression level of ITGB4, PXN, or both. In aspects, the method further comprises administering to the subject an effective amount of an ITGB4/PXN inhibitor, a Wnt/β-catenin pathway inhibitor, and a KRAS inhibitor when the subject has an elevated expression level of ITGB4, PXN, or both. In aspects, the method further comprises administering to the subject an effective amount of a KRAS inhibitor when the subject does not have an elevated expression level of ITGB4, PXN, or both; optionally monitoring the subject for treatment resistance to the KRAS inhibitor; and optionally further administering to the subject an effective amount of an ITGB4/PXN inhibitor in addition to the KRAS inhibitor when the subject is treatment resistant to the KRAS inhibitor. In aspects, the method further comprises administering to the subject an effective amount of a KRAS inhibitor when the subject does not have an elevated expression level of ITGB4, PXN, or both; optionally monitoring the subject for treatment resistance to the KRAS inhibitor; and optionally further administering to the subject an effective amount of an ITGB4/PXN inhibitor and a Wnt/r3-catenin pathway inhibitor in addition to the KRAS inhibitor when the subject is treatment resistant to the KRAS inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods of detecting resistance to cancer treatment with a KRAS inhibitor in a subject in need thereof, the method comprising: (i) measuring the expression level of Wnt, β-catenin, or both in a biological sample obtained from the subject; (ii) comparing the expression level of Wnt, β-catenin, or both in the biological sample obtained from the subject to the expression level of Wnt, β-catenin, or both in a control; wherein an elevated expression level of Wnt, β-catenin, or both indicates the subject is resistant to treatment with the KRAS inhibitor. In aspects, the method comprises measuring the expression level of Wnt in the biological sample and comparing the expression level of Wnt obtained from the subject to the control. In aspects, the method comprises measuring the expression level of β-catenin in the biological sample and comparing the expression level of β-catenin obtained from the subject to the control. In aspects, the method comprises measuring the expression level of Wnt and β-catenin in the biological sample and comparing the expression level of Wnt and β-catenin obtained from the subject to the control. In aspects, the method further comprises administering to the subject an effective amount of a Wnt/β-catenin pathway inhibitor and a KRAS inhibitor when the subject has an elevated expression level of Wnt, β-catenin, or both. In aspects, the method further comprises administering to the subject an effective amount of a Wnt/β-catenin pathway inhibitor, an ITGB4/PXN inhibitor, and a KRAS inhibitor when the subject has an elevated expression level of Wnt, β-catenin, or both. In aspects, the method further comprises administering to the subject an effective amount of a KRAS inhibitor when the subject does not have an elevated expression level of Wnt, β-catenin, or both; optionally monitoring the subject for treatment resistance to the KRAS inhibitor; and optionally further administering to the subject an effective amount of a Wnt/β-catenin pathway inhibitor in addition to the KRAS inhibitor when the subject is treatment resistant to the KRAS inhibitor. In aspects, the method further comprises administering to the subject an effective amount of a KRAS inhibitor when the subject does not have an elevated expression level of Wnt, β-catenin, or both; optionally monitoring the subject for treatment resistance to the KRAS inhibitor; and optionally further administering to the subject an effective amount of a Wnt/β-catenin pathway inhibitor and a ITGB4/PXN inhibitor in addition to the KRAS inhibitor when the subject is treatment resistant to the KRAS inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods of detecting resistance to cancer treatment with a KRAS inhibitor in a subject in need thereof, the method comprising: (i) measuring the expression level of ITGB4, PXN, Wnt, β-catenin, or a combination of two or more thereof in a biological sample obtained from the subject; (ii) comparing the expression level of ITGB4, PXN, Wnt, β-catenin, or a combination of two or more thereof in the biological sample obtained from the subject to the expression level of ITGB4, PXN, Wnt, β-catenin, or a combination of two or more thereof in a control; wherein an elevated expression level of ITGB4, PXN, Wnt, β-catenin, or a combination of two or more thereof indicates the subject is resistant to treatment with the KRAS inhibitor. In aspects, the method comprises measuring the expression level of ITGB4 and Wnt in the biological sample and comparing the expression level of ITGB4 and Wnt obtained from the subject to the control. In aspects, the method comprises measuring the expression level of ITGB4, Wnt, and β-catenin in the biological sample and comparing the expression level of ITGB4, Wnt, and β-catenin obtained from the subject to the control. In aspects, the method comprises measuring the expression level of PXN and Wnt in the biological sample and comparing the expression level of PXN and Wnt obtained from the subject to the control. In aspects, the method comprises measuring the expression level of PXN, Wnt, and β-catenin in the biological sample and comparing the expression level of PXN, Wnt, and β-catenin obtained from the subject to the control. In aspects, the method comprises measuring the expression level of ITGB4 and β-catenin in the biological sample and comparing the expression level of ITGB4 and β-catenin obtained from the subject to the control. In aspects, the method comprises measuring the expression level of ITGB4, PXN, and β-catenin in the biological sample and comparing the expression level of ITGB4, PXN, and β-catenin obtained from the subject to the control. In aspects, the method comprises measuring the expression level of PXN and β-catenin in the biological sample and comparing the expression level of PXN and β-catenin obtained from the subject to the control. In aspects, the method comprises measuring the expression level of ITGB4, PXN, Wnt, and β-catenin in the biological sample and comparing the expression level of ITGB4, PXN, Wnt, and β-catenin obtained from the subject to the control. In aspects, the method further comprises administering to the subject an effective amount of a Wnt/β-catenin pathway inhibitor and a KRAS inhibitor when the subject has an elevated expression level of ITGB4, PXN, Wnt, β-catenin, or a combination of two or more thereof. In aspects, the method further comprises administering to the subject an effective amount of a ITGB4/PXN inhibitor and a KRAS inhibitor when the subject has an elevated expression level of ITGB4, PXN, Wnt, β-catenin, or a combination of two or more thereof. In aspects, the method further comprises administering to the subject an effective amount of a Wnt/β-catenin pathway inhibitor, an ITGB4/PXN inhibitor, and a KRAS inhibitor when the subject has an elevated expression level of Wnt, β-catenin, or both. In aspects, the method further comprises administering to the subject an effective amount of a KRAS inhibitor when the subject does not have an elevated expression level of ITGB4, PXN, Wnt, β-catenin, or a combination of two or more thereof optionally monitoring the subject for treatment resistance to the KRAS inhibitor; and optionally further administering to the subject an effective amount of a Wnt/β-catenin pathway inhibitor in addition to the KRAS inhibitor when the subject is treatment resistant to the KRAS inhibitor. In aspects, the method further comprises administering to the subject an effective amount of a KRAS inhibitor when the subject does not have an elevated expression level of ITGB4, PXN, Wnt, β-catenin, or a combination of two or more thereof optionally monitoring the subject for treatment resistance to the KRAS inhibitor; and optionally further administering to the subject an effective amount of a ITGB4/PXN inhibitor in addition to the KRAS inhibitor when the subject is treatment resistant to the KRAS inhibitor. In aspects, the method further comprises administering to the subject an effective amount of a KRAS inhibitor when the subject does not have an elevated expression level of ITGB4, PXN, Wnt, β-catenin, or a combination of two or more thereof optionally monitoring the subject for treatment resistance to the KRAS inhibitor; and optionally further administering to the subject an effective amount of a Wnt/β-catenin pathway inhibitor and a ITGB4/PXN inhibitor in addition to the KRAS inhibitor when the subject is treatment resistant to the KRAS inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods to treat cancer in a subject in need thereof, the method comprising: (i) identifying a homozygous KRAS mutation in a biological sample obtained from the subject; (ii) administering an effective amount of ITGB4/PXN inhibitor and an effective amount of KRAS inhibitor to the subject having the homozygous KRAS mutation. In embodiments, the methods further comprise administering to the subject an effective amount of a Wnt/β-catenin pathway inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods to treat cancer in a subject in need thereof, the method comprising: (i) identifying a homozygous KRAS mutation in a biological sample obtained from the subject; (ii) administering an effective amount of a Wnt/β-catenin pathway inhibitor and an effective amount of KRAS inhibitor to the subject having the homozygous KRAS mutation.

In embodiments, the disclosure provides methods to treat cancer in a subject in need thereof by (i) identifying a heterozygous KRAS mutation in a biological sample obtained from a subject; (ii) administering an effective amount of KRAS inhibitor to the subject having the heterozygous KRAS mutation. In aspects, the methods further comprise monitoring the subject for treatment resistance to the KRAS inhibitor. In aspects, the methods further comprise administering to the subject an effective amount of an ITGB4/PXN inhibitor when the subject is treatment resistant to the KRAS inhibitor. Thus, the patient will be administered an effective amount of an ITGB4/PXN inhibitor and an effective amount of a KRAS inhibitor when the subject is treatment resistant to the KRAS inhibitor. In embodiments, the methods further comprise administering to the subject an effective amount of a Wnt/β-catenin pathway inhibitor. In aspects, the ITGB4/PXN inhibitor is a ITGB4/PXN pathway inhibitor. In aspects, the KRAS inhibitor is a KRAS pathway inhibitor. In aspects, the ITGB4/PXN inhibitor is a ITGB4/PXN pathway inhibitor and the KRAS inhibitor is a KRAS pathway inhibitor.

In embodiments, the disclosure provides methods to treat cancer in a subject in need thereof by (i) identifying a heterozygous KRAS mutation in a biological sample obtained from a subject; (ii) administering an effective amount of KRAS inhibitor to the subject having the heterozygous KRAS mutation. In aspects, the methods further comprise monitoring the subject for treatment resistance to the KRAS inhibitor. In aspects, the methods further comprise administering to the subject an effective amount of a Wnt/β-catenin pathway inhibitor when the subject is treatment resistant to the KRAS inhibitor. Thus, the patient will be administered an effective amount of a Wnt/β-catenin pathway inhibitor and an effective amount of a KRAS inhibitor when the subject is treatment resistant to the KRAS inhibitor.

In embodiments of the methods described herein, the cancer has a KRAS mutation. In aspects, the subject is resistant to treatment with a KRAS inhibitor. In aspects, the KRAS mutation is heterozygous. In aspects, the KRAS mutation is homozygous. In aspects, the KRAS mutation is a codon 12 mutation. In aspects, the KRAS mutation is a KRAS G12 mutation. In aspects, the KRAS mutation is a KRAS G12C mutation. In aspects, the KRAS mutation is a KRAS G12A mutation. In aspects, the KRAS mutation is a KRAS G12V mutation. In aspects, the KRAS mutation is a KRAS G12D mutation. In aspects, the KRAS mutation is a codon 13 mutation. In aspects, the KRAS mutation is a KRAS G13 mutation. In aspects, the KRAS mutation is a codon 61 mutation. In aspects, the KRAS mutation is a KRAS H61 mutation.

In embodiments of the methods described herein, the ITGB4/PXN inhibitor is any ITGB4/PXN inhibitor known in the art. In embodiments, the ITGB4/PXN inhibitor is a ITGB4/PXN pathway inhibitor. Methods to identify ITGB4/PXN inhibitors are known in the art and described herein. In aspects, the ITGB4/PXN inhibitor is carfilzomib, tozasertib, dasatinib, afatinib, dacomitinib, poziotinib, pacritinib, ixazomib, osimertinib, CUDC-101, or cerdulatinib. In aspects, the ITGB4/PXN inhibitor is carfilzomib, ixazomib, or CUDC-101. In aspects, the ITGB4/PXN inhibitor is carfilzomib. In aspects, the ITGB4/PXN inhibitor is ixazomib. In aspects, the ITGB4/PXN inhibitor is CUDC-101. In aspects, the ITGB4/PXN inhibitor is tozasertib. In aspects, the ITGB4/PXN inhibitor is dasatinib. In aspects, the ITGB4/PXN inhibitor is afatinib. In aspects, the ITGB4/PXN inhibitor is dacomitinib. In aspects, the ITGB4/PXN inhibitor is poziotinib. In aspects, the ITGB4/PXN inhibitor is pacritinib. In aspects, the ITGB4/PXN inhibitor is osimertinib. In aspects, the ITGB4/PXN inhibitor is cerdulatinib.

In embodiments of the methods described herein, the Wnt/β-catenin pathway inhibitor is any Wnt/β-catenin pathway inhibitor known in the art. Methods to identify Wnt/β-catenin pathway inhibitor are known in the art and described herein. In aspects, the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, IC-2, JIB-04, FH535, a docataxel/sulforaphane combination, pyrvinium pamoate, SKL2001, or a combination of two or more thereof. In aspects, the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, JIB-04, FH535, a docataxel/sulforaphane combination, pyrvinium pamoate, SKL2001, or a combination of two or more thereof. In aspects, the Wnt/β-catenin pathway inhibitor is tegatrabetan. In aspects, the Wnt/β-catenin pathway inhibitor is LF3. In aspects, the Wnt/β-catenin pathway inhibitor is KYA1797K. In aspects, the Wnt/β-catenin pathway inhibitor is KY1220. In aspects, the Wnt/β-catenin pathway inhibitor is a KYA1797K/KY1220 combination. In aspects, the Wnt/β-catenin pathway inhibitor is iCRT3. In aspects, the Wnt/β-catenin pathway inhibitor is iCRT5. In aspects, the Wnt/β-catenin pathway inhibitor is a iCRT3/iCRT5 combination. In aspects, the Wnt/β-catenin pathway inhibitor is iCRT14. In aspects, the Wnt/β-catenin pathway inhibitor is ZINC02092166. In aspects, the Wnt/β-catenin pathway inhibitor is NLS-StAx-h. In aspects, the Wnt/β-catenin pathway inhibitor is foscenvivint. In aspects, the Wnt/β-catenin pathway inhibitor is tabituximab barzuxetan. In aspects, the Wnt/β-catenin pathway inhibitor is vantictumab. In aspects, the Wnt/β-catenin pathway inhibitor is ipafricept. In aspects, the Wnt/β-catenin pathway inhibitor is Fz7-21. In aspects, the Wnt/β-catenin pathway inhibitor is salinomycin. In aspects, the Wnt/β-catenin pathway inhibitor is FJP. In aspects, the Wnt/β-catenin pathway inhibitor is BML-286. In aspects, the Wnt/β-catenin pathway inhibitor is XAV939. In aspects, the Wnt/β-catenin pathway inhibitor is JW74. In aspects, the Wnt/β-catenin pathway inhibitor is JW55. In aspects, the Wnt/β-catenin pathway inhibitor is NVP-TNKS656. In aspects, the Wnt/β-catenin pathway inhibitor is LZZ-02. In aspects, the Wnt/β-catenin pathway inhibitor is SSTC3. In aspects, the Wnt/β-catenin pathway inhibitor is WNT974. In aspects, the Wnt/β-catenin pathway inhibitor is ETC-159. In aspects, the Wnt/β-catenin pathway inhibitor is CGX1321. In aspects, the Wnt/β-catenin pathway inhibitor is GNF-6231. In aspects, the Wnt/β-catenin pathway inhibitor is ICG001. In aspects, the Wnt/β-catenin pathway inhibitor is isoquercitrin. In aspects, the Wnt/β-catenin pathway inhibitor is FJ9. In aspects, the Wnt/β-catenin pathway inhibitor is IWP-2. In aspects, the Wnt/β-catenin pathway inhibitor is IWP-4. In aspects, the Wnt/β-catenin pathway inhibitor is calphostin C. In aspects, the Wnt/β-catenin pathway inhibitor is DKN-01. In aspects, the Wnt/β-catenin pathway inhibitor is CWP291. In aspects, the Wnt/β-catenin pathway inhibitor is GNE-781. In aspects, the Wnt/β-catenin pathway inhibitor is Wnt-059. In aspects, the Wnt/β-catenin pathway inhibitor is endo-IWR-1. In aspects, the Wnt/β-catenin pathway inhibitor is niclosamide. In aspects, the Wnt/β-catenin pathway inhibitor is ONC201. In aspects, the Wnt/β-catenin pathway inhibitor is tri(2-furyl)phosphine. In aspects, the Wnt/β-catenin pathway inhibitor is a actinomycin D/telmisartan combination. In aspects, the Wnt/β-catenin pathway inhibitor is chelerythrine. In aspects, the Wnt/β-catenin pathway inhibitor is IC-2. In aspects, the Wnt/β-catenin pathway inhibitor is JIB-04. In aspects, the Wnt/β-catenin pathway inhibitor is FH535. In aspects, the Wnt/β-catenin pathway inhibitor is a docataxel/sulforaphane combination. In aspects, the Wnt/β-catenin pathway inhibitor is pyrvinium pamoate. In aspects, the Wnt/β-catenin pathway inhibitor is SKL2001.

In embodiments of the methods described herein, the KRAS inhibitor can be any KRAS inhibitor known in the art. In aspects, the KRAS inhibitor is a KRAS pathway inhibitor. In aspects, the KRAS inhibitor is a pan-KRAS inhibitor. In aspects, the KRAS inhibitor is a KRAS G12 inhibitor. In aspects, the KRAS inhibitor is a KRAS G12C inhibitor. In aspects, the KRAS inhibitor is a KRAS G12A inhibitor. In aspects, the KRAS inhibitor is a KRAS G12V inhibitor. In aspects, the KRAS inhibitor is a KRAS G12D inhibitor. In aspects, the KRAS inhibitor is a KRAS G13 inhibitor. In aspects, the KRAS inhibitor is a KRAS G12 and KRAS G13 inhibitor. In aspects, the KRAS inhibitor is a KRAS H61 inhibitor. In aspect, the KRAS inhibitor is sotorasib, adagrasib, MRTX1133, BI-1701963, BI-3406, compound 0375-0604, LY3499446, JNJ-74699157, TNO155, LY3537982, or ARS1620. In aspect, the KRAS inhibitor is sotorasib, adagrasib, BI-1701963, BI-3406, compound 0375-0604, LY3499446, JNJ-74699157, TNO155, LY3537982, or ARS1620. In aspect, the KRAS inhibitor is sotorasib, adagrasib, MRTX1133, BI-1701963, BI-3406, compound 0375-0604, LY3499446, JNJ-74699157, TNO155, LY3537982, or ARS1620. In aspect, the KRAS inhibitor is sotorasib. In aspect, the KRAS inhibitor is adagrasib. In aspect, the KRAS inhibitor is BI-1701963. In aspect, the KRAS inhibitor is BI-3406. In aspects, the KRAS inhibitor is compound 0375-0604. In aspects, the KRAS inhibitor is LY3499446. In aspects, the KRAS inhibitor is JNJ-74699157. In aspects, the KRAS inhibitor is TNO155. In aspects, the KRAS inhibitor is LY3537982. In aspects, the KRAS inhibitor is ARS1620. In aspects, the KRAS inhibitor is MRTX1133. Methods of identifying KRAS mutations in a cancer are known in the art.

In embodiments of the methods described herein, the cancer can be any cancer that has a KRAS mutation. In aspect, the cancer is melanoma, ovarian cancer, endometrial cancer, lung cancer, colorectal cancer, colon cancer, or pancreatic cancer. In aspect, the cancer is ovarian cancer, lung cancer, colorectal cancer, colon cancer, or pancreatic cancer. In aspect, the cancer is lung cancer, colorectal cancer, colon cancer, or pancreatic cancer. In aspect, the cancer is non-small cell lung cancer, colorectal cancer, colon cancer, or pancreatic cancer. In aspect, the cancer is lung cancer. In aspect, the cancer is primary lung cancer. In aspect, the cancer is metastatic lung cancer. In aspect, the lung cancer is non-small cell lung cancer. In aspect, the lung cancer is primary non-small cell lung cancer. In aspect, the lung cancer is metastatic non-small cell lung cancer. In aspects, the cancer is colorectal cancer. In aspects, the cancer is primary colorectal cancer. In aspects, the cancer is metastatic colorectal cancer. In aspects, the cancer is colon cancer. In aspects, the cancer is primary colon cancer. In aspects, the cancer is metastatic colon cancer. In aspects, the cancer is pancreatic cancer. In aspects, the cancer is primary pancreatic cancer. In aspects, the cancer is metastatic pancreatic cancer. In aspects, the cancer is melanoma. In aspects, the cancer is ovarian cancer. In aspects, the cancer is endometrial cancer. In aspects, the cancer is uterine cancer. In aspects, the cancer is cervical cancer. In aspects, the cancer is breast cancer. In aspects, the cancer is prostate cancer.

The disclosure provides methods of treating cancer in a subject in need thereof by administering to the subject an effective amount of an ITGB4/PXN pathway inhibitor and an effective amount of sotorasib; wherein the cancer has a KRAS G12 mutation. In aspects, the methods of treating cancer comprise administering to the subject an effective amount of an ITGB4/PXN pathway inhibitor selected from the group consisting of carfilzomib, tozasertib, dasatinib, afatinib, dacomitinib, poziotinib, pacritinib, ixazomib, osimertinib, 7-((4-(3-ethynylphenyl)-amino)-7-methoxyquinazolin-6-yl)oxy)-N-hydroxyheptanamide, and cerdulatinib, and an effective amount of sotorasib; wherein the cancer has a KRAS G12 mutation. In aspects, the methods of treating cancer comprise administering to the subject an effective amount of carfilzomib and an effective amount of sotorasib; wherein the cancer has a KRAS G12 mutation. In aspects, the methods of treating cancer comprise administering to the subject an effective amount of tozasertib and an effective amount of sotorasib; wherein the cancer has a KRAS G12 mutation. In aspects, the methods of treating cancer comprise administering to the subject an effective amount of dasatinib and an effective amount of sotorasib; wherein the cancer has a KRAS G12 mutation. In aspects, the methods of treating cancer comprise administering to the subject an effective amount of afatinib and an effective amount of sotorasib; wherein the cancer has a KRAS G12 mutation. In aspects, the methods of treating cancer comprise administering to the subject an effective amount of dacomitinib and an effective amount of sotorasib; wherein the cancer has a KRAS G12 mutation. In aspects, the methods of treating cancer comprise administering to the subject an effective amount of poziotinib and an effective amount of sotorasib; wherein the cancer has a KRAS G12 mutation. In aspects, the methods of treating cancer comprise administering to the subject an effective amount of pacritinib and an effective amount of sotorasib; wherein the cancer has a KRAS G12 mutation. In aspects, the methods of treating cancer comprise administering to the subject an effective amount of ixazomib and an effective amount of sotorasib; wherein the cancer has a KRAS G12 mutation. In aspects, the methods of treating cancer comprise administering to the subject an effective amount of osimertinib and an effective amount of sotorasib; wherein the cancer has a KRAS G12 mutation. In aspects, the methods of treating cancer comprise administering to the subject an effective amount of cerdulatinib and an effective amount of sotorasib; wherein the cancer has a KRAS G12 mutation. In aspects, the methods of treating cancer comprise administering to the subject an effective amount of 7-((4-((3-ethynylphenyl)amino)-7-methoxyquinazolin-6-yl)oxy)-N-hydroxyheptanamide and an effective amount of sotorasib; wherein the cancer has a KRAS G12 mutation. In aspects, the KRAS mutation is a KRAS G12 mutation is a KRAS G12C mutation. In aspects, the cancer is non-small cell lung cancer. In aspects, the cancer is colorectal cancer, colon cancer, or pancreatic cancer.

The disclosure provides methods of treating cancer in a subject in need thereof by administering to the subject an effective amount of an ITGB4/PXN pathway inhibitor and an effective amount of adagrasib; wherein the cancer has a KRAS G12 mutation. In aspects, the methods of treating cancer comprise administering to the subject an effective amount of an ITGB4/PXN pathway inhibitor selected from the group consisting of carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, and cerdulatinib, and an effective amount of adagrasib; wherein the cancer has a KRAS G12 mutation. In aspects, the methods of treating cancer comprise administering to the subject an effective amount of carfilzomib and an effective amount of adagrasib; wherein the cancer has a KRAS G12 mutation. In aspects, the methods of treating cancer comprise administering to the subject an effective amount of tozasertib and an effective amount of adagrasib; wherein the cancer has a KRAS G12 mutation. In aspects, the methods of treating cancer comprise administering to the subject an effective amount of dasatinib and an effective amount of adagrasib; wherein the cancer has a KRAS G12 mutation. In aspects, the methods of treating cancer comprise administering to the subject an effective amount of afatinib and an effective amount of adagrasib; wherein the cancer has a KRAS G12 mutation. In aspects, the methods of treating cancer comprise administering to the subject an effective amount of dacomitinib and an effective amount of adagrasib; wherein the cancer has a KRAS G12 mutation. In aspects, the methods of treating cancer comprise administering to the subject an effective amount of poziotinib and an effective amount of adagrasib; wherein the cancer has a KRAS G12 mutation. In aspects, the methods of treating cancer comprise administering to the subject an effective amount of pacritinib and an effective amount of adagrasib; wherein the cancer has a KRAS G12 mutation. In aspects, the methods of treating cancer comprise administering to the subject an effective amount of ixazomib and an effective amount of adagrasib; wherein the cancer has a KRAS G12 mutation. In aspects, the methods of treating cancer comprise administering to the subject an effective amount of osimertinib and an effective amount of adagrasib; wherein the cancer has a KRAS G12 mutation. In aspects, the methods of treating cancer comprise administering to the subject an effective amount of cerdulatinib and an effective amount of adagrasib; wherein the cancer has a KRAS G12 mutation. In aspects, the methods of treating cancer comprise administering to the subject an effective amount of 7-((4-((3-ethynylphenyl)-amino)-7-methoxyquinazolin-6-yl)oxy)-N-hydroxyheptanamide and an effective amount of adagrasib; wherein the cancer has a KRAS G12 mutation. In aspects, the KRAS G12 mutation is a KRAS G12C mutation. In aspects, the cancer is non-small cell lung cancer. In aspects, the cancer is colorectal cancer, colon cancer, or pancreatic cancer.

Dose and Dosing Regimens

The dosage and frequency (single or multiple doses) of the ITGB4/PXN inhibitor, Wnt/β-catenin pathway inhibitor, and KRAS inhibitor administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and ITGB4/PXN inhibitors, Wnt/β-catenin pathway inhibitors, and KRAS inhibitors described herein. Adjustment and manipulation of established dosages (e.g., frequency and duration) are well within the ability of those skilled in the art.

For any composition and ITGB4/PXN inhibitor, Wnt/β-catenin pathway inhibitor, and KRAS inhibitor described herein, the therapeutically effective amount can be initially determined from cell culture assays. Target concentrations will be those concentrations of ITGB4/PXN inhibitor, Wnt/β-catenin pathway inhibitor, and KRAS inhibitor that are capable of achieving the methods described herein, as measured using the methods described herein or known in the art. As is well known in the art, effective amounts of ITGB4/PXN inhibitor, Wnt/β-catenin pathway inhibitor, and KRAS inhibitor for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the ITGB4/PXN inhibitor, Wnt/β-catenin pathway inhibitor, and KRAS inhibitor may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the ITGB4/PXN inhibitor, Wnt/β-catenin pathway inhibitor, and KRAS inhibitor. Thereafter, the dosage is increased by small increments until the optimum effect under circumstances is reached. Dosage amounts and intervals can be adjusted individually to provide levels of the ITGB4/PXN inhibitor, Wnt/β-catenin pathway inhibitor, and KRAS inhibitor effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of ITGB4/PXN inhibitor, Wnt/β-catenin pathway inhibitor, and KRAS inhibitor by considering factors such as compound potency, relative bioavailability, patient body weight, presence and severity of adverse side effects.

Pharmaceutical Compositions

Provided herein are pharmaceutical compositions comprising an ITGB4/PXN inhibitor and a pharmaceutically acceptable excipient. Provided herein are pharmaceutical compositions comprising a KRAS inhibitor and a pharmaceutically acceptable excipient. Provided herein are pharmaceutical compositions comprising an ITGB4/PXN inhibitor, a KRAS inhibitor, and a pharmaceutically acceptable excipient. Provided herein are pharmaceutical compositions comprising an ITGB4/PXN inhibitor, a Wnt/β-catenin pathway inhibitor, a KRAS inhibitor, and a pharmaceutically acceptable excipient. Provided herein are pharmaceutical compositions comprising an ITGB4/PXN inhibitor, a Wnt/β-catenin pathway inhibitor, and a pharmaceutically acceptable excipient. Provided herein are pharmaceutical compositions comprising a Wnt/β-catenin pathway inhibitor and a pharmaceutically acceptable excipient. Provided herein are pharmaceutical compositions comprising a Wnt/β-catenin pathway inhibitor, a KRAS inhibitor, and a pharmaceutically acceptable excipient. The term “active ingredient” refers to ITGB4/PXN inhibitors, Wnt/β-catenin pathway inhibitors, and KRAS inhibitors. The provided compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

Compositions can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations of the compositions may be administered depending on the dosage and frequency as required and tolerated by the patient.

Pharmaceutical compositions provided herein include compositions wherein the active ingredient (e.g. compositions described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, the compounds described herein will contain an amount of active ingredient effective to achieve the desired result, e.g., modulating the activity of a target molecule, and/or reducing, eliminating, or slowing the progression of disease symptoms. Determination of a therapeutically effective amount of a compound described herein is well within the capabilities of those skilled in the art, especially in light of the detailed disclosure herein.

Provided compositions can include a single agent or more than one agent. The compositions for administration will commonly include an agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols, or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 1 to about 90% of the weight of the unit, or preferably between 1-60%. The amount of active compounds in such compositions is such that a suitable dosage can be obtained.

For parenteral administration in an aqueous solution, for example, the solution should be suitably buffered, and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Solvents can be sued for rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

“Pharmaceutically acceptable excipient” and “pharmaceutically acceptable carrier” refer to a substance that aids the administration of an active agent to and absorption by a subject and can be included in the compositions herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Additional Therapeutic Agents

In the provided methods of treatment, additional therapeutic agents can be used that are suitable to the disease (e.g., cancer) being treated. Thus, In aspects, the provided methods of treatment further include administering a third therapeutic agent to the subject. Suitable additional therapeutic agents include, but are not limited to analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

The additional therapeutic agent useful for the methods provided herein may be a compound, drug, antagonist, inhibitor, or modulator, having antineoplastic properties or the ability to inhibit the growth or proliferation of cells. In aspects, the additional therapeutic agent is a chemotherapeutic. “Anti-cancer agent” or “chemotherapeutic” or “chemotherapeutic agent” is used in accordance with its plain ordinary meaning and refers to a chemical composition or compound having antineoplastic properties or the ability to inhibit the growth or proliferation of cells. In aspects, the additional therapeutic agent is radiation therapy. In aspects, the additional therapeutic agent is an agent approved by the FDA or similar regulatory agency of a country other than the USA, for treating cancer.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In aspects, an anti-cancer agent is a chemotherapeutic. In aspects, an anti-cancer agent is an agent approved by the FDA or similar regulatory agency of a country other than the USA, for treating cancer. In aspects, the anti-cancer agent is not an anti-CD73 compound, such as an anti-CD73 antibody. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (GLEEVEC™), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. paclitaxel), Taxotere™, compounds comprising the taxane skeleton, Erbulozole (i.e. R-55104), Dolastatin 10 (i.e. DLS-10 and NSC-376128), mivobulin isethionate (i.e. as CI-980), vincristine, NSC-639829, discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin A1 (i.e. BTO-956 and DIME), Fijianolide B, Laulimalide, Narcosine (also known as NSC-5366), Nascapine, Hemiasterlin, Vanadocene acetylacetonate, Monsatrol, Inanocine (i.e. NSC-698666), Eleutherobins (such as Desmethyleleutherobin, Desaetyleleutherobin, Isoeleutherobin A, and Z-Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (IRESSA™), erlotinib (TARCEVA™), cetuximab (ERBUTUX™), lapatinib (TYKERB™), panitumumab (VECTIBIX™), vandetanib (CAPRELSA™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

A “combined synergistic amount” as used herein refers to the sum of a first amount (e.g., an amount of an ITGB4/PXN inhibitor) and a second amount (e.g., an amount of a KRAS inhibitor) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

A “combined additive amount” as used herein refers to the sum of a first amount (e.g., an amount of ITGB4/PXN inhibitor) and a second amount (e.g., an amount of a KRAS inhibitor) that results in an additive effect (i.e. an effect equal to the sum of the effects). Therefore, the terms “additive”, “combined additive amount”, and “additive therapeutic effect” which are used herein interchangeably, refer to a measured effect of compounds administered in combination where the measured effect is equal to the sum of the individual effects of each of the compounds administered alone as a single agent.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

The combined administrations contemplates co-administration, using separate formulations or a single pharmaceutical formulation, and consecutive administration in either order, wherein preferably there is a time period while both (or all) active agents simultaneously exert their biological activities.

Detection, Assay, and Diagnostic Methods

In embodiments, methods described herein may include detecting a expression level of ITGB4, PXN, or both, e.g., with a specific binding agent (e.g., an agent that binds to a protein or nucleic acid molecule). In embodiments, methods described herein may include detecting a expression level of ITGB4, PXN, Wnt, β-catenin, or a combination of two or more thereof, e.g., with a specific binding agent (e.g., an agent that binds to a protein or nucleic acid molecule). In embodiments, methods described herein may include detecting a expression level of Wnt, β-catenin, or both, e.g., with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide comprising a sequence that is complementary to patient genomic DNA, mRNA or a cDNA produced from patient mRNA, or any combination thereof. In aspects, an antibody is labeled with detectable moiety, e.g., a fluorescent compound, an enzyme or functional fragment thereof, or a radioactive agent. In aspects, an antibody is detectably labeled by coupling it to a chemiluminescent compound. In aspects, the presence of the chemiluminescent-tagged antibody is then determined by detecting the presence of luminescence that arises during the course of chemical reaction. Non-limiting examples of particularly useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomolecules present in the sample do not significantly bind to the binding agent specific for the target molecule. In aspects, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻⁹) or pico molar (10⁻¹²) range for its specific target ligand.

In embodiments, the present subject matter provides a composition comprising a binding agent, wherein the binding agent is attached to a solid support, (e.g., a strip, a polymer, a bead, a nanoparticle, a plate such as a multiwell plate, or an array such as a microarray). In aspects relating to the use of a nucleic acid probe attached to a solid support (such as a microarray), a nucleic acid in a test sample may be amplified (e.g., using PCR) before or after the nucleic acid to be measured is hybridized with the probe. In aspects, reverse transcription polymerase chain reaction (RT-PCR) is used to detect mRNA levels. In aspects, a probe on a solid support is used, and mRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In aspects, a strip may be a nucleic acid-probe coated porous or non-porous solid support strip comprising linking a nucleic acid probe to a carrier to prepare a conjugate and immobilizing the conjugate on a porous solid support. In aspects, the support or carrier comprises glass, polystyrene, polypropylene, polyethylene, dextran, nylon, amylases, natural and modified celluloses, polyacrylamides, gabbros, and magnetite. In aspects, the nature of the carrier can be either soluble to some extent or insoluble for the purposes of the present subject matter. In aspects, the support material may have any structural configuration so long as the coupled molecule is capable of binding to a binding agent (e.g., an antibody). In aspects, the support configuration may be spherical, as in a bead, or cylindrical, as in the inside surface of a test tube, or the external surface of a rod. In aspects, the surface may be flat such as a plate (or a well within a multiwell plate), sheet, or test strip, etc. polystyrene beads. The skilled artisan will know many other suitable carriers for binding antibody or antigen, or will be able to ascertain the same by use of routine experimentation.

In embodiments, a solid support comprises a polymer, to which an agent is chemically bound, immobilized, dispersed, or associated. In aspects, a polymer support may be, e.g., a network of polymers, and may be prepared in bead form (e.g., by suspension polymerization). In aspects, the location of active sites introduced into a polymer support depends on the type of polymer support. In aspects, in a swollen-gel-bead polymer support the active sites are distributed uniformly throughout the beads, whereas in a macroporous-bead polymer support they are predominantly on the internal surfaces of the macropores. In aspects, the solid support, e.g., a device, may contain an ITGB4 or PXN binding agent alone or together with a binding agent for at least one, two, three or more other molecules.

In aspects, detection is accomplished using an ELISA or Western blot format. In aspects, the binding agent comprises an nucleic acid (e.g., a probe or primers that are complementary for mRNA or cDNA), and the detecting step is accomplished using a polymerase chain reaction (PCR) or Northern blot format, or other means of detection. In aspects, a probe or primer is about 10-20, 15-25, 15-35, 15-25, 20-80, 50-100, or 10-100 nucleotides in length, e.g., about 10, 12, 15, 20, 25, 30, 35, 40, 45, 50, 55, 60, 70, 80, 90, or 100 nucleotides in length or less than about 30, 35, 40, 45, 50, 55, 60, 70, 80, 90, or 100 nucleotides in length.

As used herein, “assaying” or “detecting” means using an analytic procedure to qualitatively assess or quantitatively measure the presence or amount or the functional activity of a target entity (e.g., ITGB4, PXN, Wnt, β-catenin). For example, assaying the level of a compound (such as a protein or an mRNA molecule) means using an analytic procedure (such as an in vitro procedure) to qualitatively assess or quantitatively measure the presence or amount of the compound.

In embodiments, the cells in a biological sample are lysed to release a protein or nucleic acid. Numerous methods for lysing cells and assessing protein and nucleic acid levels are known in the art. In aspects, cells are physically lysed, such as by mechanical disruption, liquid homogenization, high frequency sound waves, freeze/thaw cycles, with a detergent, or manual grinding. Non-limiting examples of detergents include Tween 20, Triton X-100, and Sodium Dodecyl Sulfate (SDS). Non-limiting examples of assays for determining the level of a protein include HPLC, LC/MS, ELISA, immunoelectrophoresis, Western blot, immunohistochemistry, and radioimmuno assays. Non-limiting examples of assays for determining the level of an mRNA include Northern blotting, RT-PCR, RNA sequencing, and qRT-PCR.

In embodiments, a cancer tumor sample can be obtained by a variety of procedures including, but not limited to, surgical excision, aspiration, or biopsy. In aspects, the tissue sample may be sectioned and assayed as a fresh specimen; alternatively, the tissue sample may be frozen for further sectioning. In aspects, the tissue sample is preserved by fixing and embedding in paraffin or the like.

In embodiments, once a suitable biological sample (e.g., cancer tumor) has been obtained, it is analyzed to quantitate the expression level of each of the genes, e.g., ITGB4, PXN, Wnt, β-catenin. In aspects, determining the expression level of a gene comprises detecting and quantifying RNA transcribed from that gene or a protein translated from such RNA. In aspects, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In aspects, when the gene expression is detected using the nCounter® Analysis System marketed by NanoString® Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In aspects, the T-effector signature score is then calculated as the arithmetic mean of normalized values for the genes in the gene signature.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

### Embodiments P1-P44

Embodiment P1. A method of treating cancer in a subject in need thereof, the method comprising administering to the subject an effective amount of an ITGB4/PXN inhibitor and an effective amount of a KRAS inhibitor; wherein the cancer has a KRAS mutation.

Embodiment P2. The method of Embodiment P1, wherein the subject is resistant to treatment with a KRAS inhibitor.

Embodiment P3. A method to select a subject with cancer for treatment with a ITGB4/PXN inhibitor and a KRAS inhibitor, the method comprising measuring a KRAS mutation in a biological sample obtained from the subject; wherein if the KRAS mutation is present in the biological sample, the subject is selected for treatment with the ITGB4/PXN inhibitor and the KRAS inhibitor.

Embodiment P4. A method of detecting resistance to cancer treatment with a KRAS inhibitor in a subject in need thereof, the method comprising: (i) measuring the expression level of ITGB4, PXN, or both in a biological sample obtained from the subject; (ii) comparing the expression level of ITGB4, PXN, or both in the biological sample obtained from the subject to the expression level of ITGB4, PXN, or both in a control; wherein an elevated expression level of ITGB4, PXN, or both indicates the subject is resistant to treatment with the KRAS inhibitor.

Embodiment P5. The method of Embodiment P4, further comprising administering to the subject an effective amount of an ITGB4/PXN inhibitor and a KRAS inhibitor when the subject has an elevated expression level of ITGB4, PXN, or both.

Embodiment P6. The method of Embodiment P4, further comprising administering to the subject an effective amount of a KRAS inhibitor when the subject does not have an elevated expression level of ITGB4, PXN, or both.

Embodiment P7. The method of Embodiment P6, further comprising monitoring the subject for treatment resistance to the KRAS inhibitor.

Embodiment P8. The method of Embodiment P7, further comprising administering to the subject an effective amount of an ITGB4/PXN inhibitor in addition to the KRAS inhibitor when the subject is treatment resistant to the KRAS inhibitor.

Embodiment P9. The method of any one of Embodiments P1 to P8, wherein the KRAS mutation is heterozygous.

Embodiment P10. The method of any one of Embodiments P1 to P8, wherein the KRAS mutation is homozygous.

Embodiment P11. A method to treat cancer in a subject in need thereof, the method comprising: (i) identifying a homozygous KRAS mutation in a biological sample obtained from the subject; (ii) administering an ITGB4/PXN inhibitor and a KRAS inhibitor to the subject having the homozygous KRAS mutation.

Embodiment P12. A method to treat cancer in a subject in need thereof, the method comprising: (i) identifying a heterozygous KRAS mutation in a biological sample obtained from a subject; (ii) administering a KRAS inhibitor to the subject having the heterozygous KRAS mutation.

Embodiment P13. The method of Embodiment P12, further comprising monitoring the subject for treatment resistance to the KRAS inhibitor.

Embodiment P14. The method of Embodiment P13, further comprising administering to the subject an effective amount of an ITGB4/PXN inhibitor when the subject is treatment resistant to the KRAS inhibitor.

Embodiment P15. The method of any one of Embodiments P1 to P14, wherein the KRAS mutation is a KRAS G12 mutation.

Embodiment P16. The method of Embodiment P15, wherein the KRAS G12 mutation is a KRAS G12C mutation.

Embodiment P17. The method of any one of Embodiments P1 to P14, wherein the KRAS mutation is a KRAS G12 mutation, a KRAS G13 mutation, a KRAS H61, or a combination of two or more thereof.

Embodiment P18. The method of any one of Embodiments P1 to P14, wherein the KRAS mutation is a KRAS G12C mutation, a KRAS G12A mutation, a KRAS G12V mutation, or a KRAS G12D mutation.

Embodiment P19. The method of any one of Embodiments P1 to P18, wherein the ITGB4/PXN inhibitor is carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, or cerdulatinib.

Embodiment P20. The method of Embodiment P19, wherein the ITGB4/PXN inhibitor is carfilzomib, ixazomib, or 7-((4-((3-ethynylphenyl)amino)-7-methoxyquinazolin-6-yl)oxy)-N-hydroxyheptanamide.

Embodiment P21. The method of Embodiment P20, wherein the ITGB4/PXN inhibitor is carfilzomib.

Embodiment P22. The method of any one of Embodiments P1 to P21, wherein the KRAS inhibitor is a KRAS G12 inhibitor.

Embodiment P23. The method of Embodiment P22, wherein the KRAS inhibitor is KRAS G12C inhibitor.

Embodiment P24. The method of any one of Embodiments P1 to P21, wherein the KRAS inhibitor is a pan-KRAS inhibitor.

Embodiment P25. The method of any one of Embodiments P1 to P21, wherein the KRAS inhibitor is a KRAS G12 inhibitor, a KRAS G13 inhibitor, or a combination thereof.

Embodiment P26. The method of any one of Embodiments P1 to P21, wherein the KRAS inhibitor is sotorasib.

Embodiment P27. The method of any one of Embodiments P1 to P21, wherein the KRAS inhibitor is adagrasib.

Embodiment P28. The method of any one of Embodiments P1 to P21, wherein the KRAS inhibitor is BI-1701963 or N-((R)-1-(3-amino-5-(trifluoromethyl)phenyl)ethyl)-7-methoxy-2-methyl-6-(((S)-tetrahydrofuran-3-yl)oxy)quinazolin-4-amine.

Embodiment P29. The method of any one of Embodiments P1 to P21, wherein the KRAS inhibitor is sotorasib, adagrasib, BI-1701963, N-((R)-1-(3-amino-5-(trifluoromethyl)-phenyl)ethyl)-7-methoxy-2-methyl-6-(((S)-tetrahydrofuran-3-yl)oxy)quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one.

Embodiment P30. The method of any one of Embodiments P1 to P29, wherein the cancer is lung cancer.

Embodiment P31. The method of Embodiment P30, wherein the lung cancer is non-small cell lung cancer.

Embodiment P32. The method of any one of Embodiments P1 to P29, wherein the cancer is colorectal cancer, colon cancer, or pancreatic cancer.

Embodiment P33. The method of any one of Embodiments P1 to P32, wherein the cancer is a primary cancer or a metastatic cancer.

Embodiment P34. A pharmaceutical composition comprising a ITGB4/PXN inhibitor, a KRAS inhibitor, and a pharmaceutically acceptable carrier.

Embodiment P35. The pharmaceutical composition of Embodiment P34, wherein the ITGB4/PXN inhibitor is carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, or cerdulatinib.

Embodiment P36. The pharmaceutical composition of Embodiment P35, wherein the ITGB4/PXN inhibitor is carfilzomib, ixazomib, or 7-((4-((3-ethynylphenyl)amino)-7-methoxyquinazolin-6-yl)oxy)-N-hydroxyheptanamide.

Embodiment P37. The pharmaceutical composition of Embodiment P36, wherein the ITGB4/PXN inhibitor is carfilzomib.

Embodiment P38. The pharmaceutical composition of any one of Embodiments P34 to P37, wherein the KRAS inhibitor is a KRAS G12 inhibitor, a KRAS G13 inhibitor, or a combination thereof.

Embodiment P39. The pharmaceutical composition of any one of Embodiments P34 to P37, wherein the KRAS inhibitor is a KRAS G12C inhibitor.

Embodiment P40. The pharmaceutical composition of any one of Embodiments P34 to P37, wherein the KRAS inhibitor is a pan-KRAS inhibitor.

Embodiment P41. The pharmaceutical composition of any one of Embodiments P34 to 37, wherein the KRAS inhibitor is sotorasib.

Embodiment P42. The pharmaceutical composition of any one of Embodiments P34 to P37, wherein the KRAS inhibitor is adagrasib.

Embodiment P43. The pharmaceutical composition of any one of Embodiments P34 to P37, wherein the KRAS inhibitor is BI-1701963 or N-((R)-1-(3-amino-5-(trifluoromethyl)-phenyl)ethyl)-7-methoxy-2-methyl-6-(((S)-tetrahydrofuran-3-yl)oxy)quinazolin-4-amine.

Embodiment P44. The pharmaceutical composition of any one of Embodiments P34 to P37, wherein the KRAS inhibitor is sotorasib, adagrasib, BI-1701963, N-((R)-1-(3-amino-5-(trifluoromethyl)phenyl)ethyl)-7-methoxy-2-methyl-6-(((S)-tetrahydrofuran-3-yl)oxy)-quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl(2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one.

Embodiment P45. The method of any one of Embodiments P1, P2, P5, P8-P11, and P14-P33, wherein the ITGB4/PXN inhibitor and the KRAS inhibitor are separately administered to the subject (i.e., administering to the subject: (a) an effective amount of a first pharmaceutical composition comprising the ITGB4/PXN inhibitor, and (b) an effective amount of a second pharmaceutical composition comprising the KRAS inhibitor).

Embodiment P45. The method of any one of Embodiments P1, P2, P5, P8-P11, and P14-P33, wherein the ITGB4/PXN inhibitor and the KRAS inhibitor are administered to the subject in a single pharmaceutical composition (i.e., administering to the subject an effective amount of a pharmaceutical composition comprising the ITGB4/PXN inhibitor, the KRAS inhibitor, and a pharmaceutically acceptable excipient (e.g., as set forth in Embodiments P34-P44)).

### Embodiments 1 to 72

Embodiment 1. A method of treating cancer in a subject in need thereof, the method comprising administering to the subject an effective amount of an ITGB4/PXN pathway inhibitor and an effective amount of a KRAS pathway inhibitor; wherein the cancer has a KRAS mutation.

Embodiment 2. The method of Embodiment 1, further comprising administering to the subject an effective amount of a Wnt/β-catenin pathway inhibitor.

Embodiment 3. A method of treating cancer in a subject in need thereof, the method comprising administering to the subject an effective amount of a Wnt/β-catenin pathway inhibitor and an effective amount of a KRAS pathway inhibitor; wherein the cancer has a KRAS mutation.

Embodiment 4. The method of any one of Embodiments 1 to 3, wherein the subject is resistant to treatment with a KRAS pathway inhibitor.

Embodiment 5. A method to select a subject with a KRAS mutation for cancer treatment, the method comprising measuring a KRAS mutation in a biological sample obtained from the subject; wherein if the KRAS mutation is present in the biological sample, the subject is selected for treatment with: (i) an ITGB4/PXN pathway inhibitor and a KRAS pathway inhibitor; (ii) a Wnt/β-catenin pathway inhibitor and a KRAS pathway inhibitor; or (iii) an ITGB4/PXN pathway inhibitor, a Wnt/β-catenin pathway inhibitor, and a KRAS pathway inhibitor.

Embodiment 6. The method of Embodiment 5, wherein the subject is selected for treatment with the ITGB4/PXN pathway inhibitor and the KRAS pathway inhibitor.

Embodiment 7. The method of Embodiment 5, wherein the subject is selected for treatment with the Wnt/β-catenin pathway inhibitor and a KRAS pathway inhibitor.

Embodiment 8. The method of Embodiment 5, wherein the subject is selected for treatment with the ITGB4/PXN pathway inhibitor, the Wnt/β-catenin pathway inhibitor, and the KRAS pathway inhibitor.

Embodiment 9. A method of detecting resistance to cancer treatment with a KRAS pathway inhibitor in a subject in need thereof, the method comprising: (i) measuring the expression level of ITGB4, PXN, or both in a biological sample obtained from the subject; (ii) comparing the expression level of ITGB4, PXN, or both in the biological sample obtained from the subject to the expression level of ITGB4, PXN, or both in a control; wherein an elevated expression level of ITGB4, PXN, or both indicates the subject is resistant to treatment with the KRAS pathway inhibitor.

Embodiment 10. The method of Embodiment 9, further comprising administering to the subject an effective amount of an ITGB4/PXN pathway inhibitor and a KRAS pathway inhibitor when the subject has an elevated expression level of ITGB4, PXN, or both.

Embodiment 11. The method of Embodiment 10, further comprising administering to the subject an effective amount of a Wnt/β-catenin pathway inhibitor.

Embodiment 12. The method of Embodiment 9, further comprising administering to the subject an effective amount of a KRAS pathway inhibitor when the subject does not have an elevated expression level of ITGB4, PXN, or both.

Embodiment 13. The method of Embodiment 12, further comprising monitoring the subject for treatment resistance to the KRAS pathway inhibitor.

Embodiment 14. The method of Embodiment 13, further comprising administering to the subject an effective amount of an ITGB4/PXN pathway inhibitor in addition to the KRAS pathway inhibitor when the subject is treatment resistant to the KRAS pathway inhibitor.

Embodiment 15. The method of Embodiment 14, further comprising administering to the subject an effective amount of a Wnt/β-catenin pathway inhibitor.

Embodiment 16. A method of detecting resistance to cancer treatment with a KRAS pathway inhibitor in a subject in need thereof, the method comprising: (i) measuring the expression level of Wnt, β-catenin, or both in a biological sample obtained from the subject; (ii) comparing the expression level of Wnt, β-catenin, or both in the biological sample obtained from the subject to the expression level of Wnt, β-catenin, or both in a control; wherein an elevated expression level of Wnt, β-catenin, or both indicates the subject is resistant to treatment with the KRAS pathway inhibitor.

Embodiment 17. The method of Embodiment 16, further comprising administering to the subject an effective amount of a Wnt/β-catenin pathway inhibitor and a KRAS pathway inhibitor when the subject has an elevated expression level of Wnt, β-catenin, or both.

Embodiment 18. The method of Embodiment 16, further comprising administering to the subject an effective amount of a KRAS pathway inhibitor when the subject does not have an elevated expression level of Wnt, β-catenin, or both.

Embodiment 19. The method of Embodiment 18, further comprising monitoring the subject for treatment resistance to the KRAS pathway inhibitor.

Embodiment 20. The method of Embodiment 19, further comprising administering to the subject an effective amount of a Wnt/β-catenin pathway inhibitor in addition to the KRAS pathway inhibitor when the subject is treatment resistant to the KRAS pathway inhibitor.

Embodiment 21. The method of any one of Embodiments 1 to 20, wherein the KRAS mutation is heterozygous.

Embodiment 22. The method of any one of Embodiments 1 to 20, wherein the KRAS mutation is homozygous.

Embodiment 23. A method to treat cancer in a subject in need thereof, the method comprising: (i) identifying a homozygous KRAS mutation in a biological sample obtained from the subject; and (ii) administering to the subject having the homozygous KRAS mutation: (a) an ITGB4/PXN pathway inhibitor and a KRAS pathway inhibitor; (b) a Wnt/β-catenin pathway inhibitor and a KRAS pathway inhibitor; or (c) an ITGB4/PXN pathway inhibitor, a Wnt/r3-catenin pathway inhibitor, and a KRAS pathway inhibitor.

Embodiment 24. The method of Embodiment 23, comprising administering the ITGB4/PXN pathway inhibitor and the KRAS pathway inhibitor.

Embodiment 25. The method of Embodiment 23, comprising administering the Wnt/r3-catenin pathway inhibitor and a KRAS pathway inhibitor.

Embodiment 26. The method of Embodiment 23, comprising administering the ITGB4/PXN pathway inhibitor, the Wnt/β-catenin pathway inhibitor, and the KRAS pathway inhibitor.

Embodiment 27. A method to treat cancer in a subject in need thereof, the method comprising: (i) identifying a heterozygous KRAS mutation in a biological sample obtained from a subject; and (ii) administering a KRAS pathway inhibitor to the subject having the heterozygous KRAS mutation.

Embodiment 28. The method of Embodiment 28, further comprising monitoring the subject for treatment resistance to the KRAS pathway inhibitor.

Embodiment 29. The method of Embodiment 28, further comprising administering to the subject an effective amount of an ITGB4/PXN pathway inhibitor when the subject is treatment resistant to the KRAS pathway inhibitor.

Embodiment 30. The method of Embodiment 28, further comprising administering to the subject an effective amount of a Wnt/β-catenin pathway inhibitor when the subject is treatment resistant to the KRAS pathway inhibitor.

Embodiment 31. The method of Embodiment 28, further comprising administering to the subject an effective amount of an ITGB4/PXN pathway inhibitor and a Wnt/β-catenin pathway inhibitor when the subject is treatment resistant to the KRAS pathway inhibitor.

Embodiment 32. The method of any one of Embodiments 1 to 31, wherein the KRAS mutation is a KRAS G12 mutation.

Embodiment 33. The method of Embodiment 32, wherein the KRAS G12 mutation is a KRAS G12C mutation.

Embodiment 34. The method of any one of Embodiments 1 to 31, wherein the KRAS mutation is a KRAS G12 mutation, a KRAS G13 mutation, a KRAS H61, or a combination of two or more thereof.

Embodiment 35. The method of any one of Embodiments 1 to 31, wherein the KRAS mutation is a KRAS G12C mutation, a KRAS G12A mutation, a KRAS G12V mutation, or a KRAS G12D mutation.

Embodiment 36. The method of any one of Embodiments 1, 2, 4-6, 8-15, 21-24, 26, 29, 31, and 32-35, wherein the ITGB4/PXN pathway inhibitor is carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, or cerdulatinib.

Embodiment 37. The method of Embodiment 36, wherein the ITGB4/PXN pathway inhibitor is carfilzomib, ixazomib, or 7-((4-((3-ethynylphenyl)amino)-7-methoxyquinazolin-6-yl)oxy)-N-hydroxyheptanamide.

Embodiment 38. The method of Embodiment 36, wherein the ITGB4/PXN pathway inhibitor is carfilzomib.

Embodiment 39. The method of any one of Embodiments 2-5, 7, 8, 11, 15-23, 25, and 30-35, wherein the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, IC-2, JIB-04, FH535, a docataxel/sulforaphane combination, pyrvinium pamoate, SKL2001, or a combination of two or more thereof.

Embodiment 40. The method of any 39, wherein the Wnt/β-catenin pathway inhibitor is tegatrabetan.

Embodiment 41. The method of any one of Embodiments 1 to 40, wherein the KRAS pathway inhibitor is a KRAS G12 inhibitor.

Embodiment 42. The method of Embodiment 41, wherein the KRAS pathway inhibitor is KRAS G12C inhibitor.

Embodiment 43. The method of any one of Embodiments 1 to 40, wherein the KRAS pathway inhibitor is a pan-KRAS inhibitor.

Embodiment 44. The method of any one of Embodiments 1 to 40, wherein the KRAS pathway inhibitor is a KRAS G12 inhibitor, a KRAS G13 inhibitor, or a combination thereof.

Embodiment 45. The method of any one of Embodiments 1 to 40, wherein the KRAS pathway inhibitor is sotorasib.

Embodiment 46. The method of any one of Embodiments 1 to 40, wherein the KRAS pathway inhibitor is adagrasib.

Embodiment 47. The method of any one of Embodiments 1 to 40, wherein the KRAS pathway inhibitor is BI-1701963 or N-((R)-1-(3-amino-5-(trifluoromethyl)phenyl)ethyl)-7-methoxy-2-methyl-6-(((S)-tetrahydrofuran-3-yl)oxy)quinazolin-4-amine.

Embodiment 48. The method of any one of Embodiments 1 to 40, wherein the KRAS pathway inhibitor is sotorasib, adagrasib, MRTX1133, ARS1620, BI-1701963, N-((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl)2-oxa-8-azaspiro[4.5]-decan-4-amine, or (S)-1-(4-(6-chloro-8-fluoro-7-(2-fluoro-6-hydroxyphenyl)-quinazolin-4-yl)piperazin-1-yl)prop-2-en-1-one.

Embodiment 49. The method of any one of Embodiments 1, 2, 4-6, 8-15, 21-24, 26, 29, 31, and 32-35, wherein the ITGB4/PXN pathway inhibitor is carfilzomib and the KRAS pathway inhibitor is sotorasib.

Embodiment 50. The method of any one of Embodiments 1, 2, 4-6, 8-15, 21-24, 26, 29, 31, and 32-35, wherein the ITGB4/PXN pathway inhibitor is carfilzomib and the KRAS pathway inhibitor is adagrasib.

Embodiment 51. The method of any one of Embodiments 1 to 50, wherein the cancer is lung cancer.

Embodiment 52. The method of Embodiment 51, wherein the lung cancer is non-small cell lung cancer.

Embodiment 53. The method of any one of Embodiments 1 to 50, wherein the cancer is colorectal cancer, colon cancer, or pancreatic cancer.

Embodiment 54. The method of any one of Embodiments 1 to 50, wherein the cancer is a solid tumor.

Embodiment 55. The method of any one of Embodiments 1 to 53, wherein the cancer is a primary cancer or a metastatic cancer.

Embodiment 56. A method of treating non-small cell lung cancer in a patient in need thereof, the method comprising administering to the subject an effective amount of carfilzomib and an effective amount of sotorasib; wherein the non-small cell lung cancer has a KRAS G12C mutation.

Embodiment 57. A method of treating non-small cell lung cancer in a patient in need thereof, the method comprising administering to the subject an effective amount of carfilzomib and an effective amount of adagrasib; wherein the non-small cell lung cancer has a KRAS G12C mutation.

Embodiment 58. A pharmaceutical composition comprising a ITGB4/PXN pathway inhibitor, a KRAS pathway inhibitor, and a pharmaceutically acceptable carrier.

Embodiment 59. The pharmaceutical composition of Embodiment 58, wherein the ITGB4/PXN pathway inhibitor is carfilzomib, tozasertib, dasatinib, afatinib, dacomitinib, poziotinib, pacritinib, ixazomib, osimertinib, 7-((4-((3-ethynylphenyl)amino)-7-methoxy-quinazolin-6-yl)oxy)-N-hydroxyheptanamide, or cerdulatinib.

Embodiment 60. The pharmaceutical composition of Embodiment 59, wherein the ITGB4/PXN pathway inhibitor is carfilzomib, ixazomib, or 7-((4-((3-ethynylphenyl)amino)-7-methoxy quinazolin-6-yl)oxy)-N-hydroxyheptanamide.

Embodiment 61. The pharmaceutical composition of Embodiment 60, wherein the ITGB4/PXN pathway inhibitor is carfilzomib.

Embodiment 62. The pharmaceutical composition of any one of Embodiments 58 to 61, further comprising a Wnt/β-catenin pathway inhibitor.

Embodiment 63. A pharmaceutical composition comprising a Wnt/β-catenin pathway inhibitor, a KRAS pathway inhibitor, and a pharmaceutically acceptable carrier.

Embodiment 64. The pharmaceutical composition of Embodiment 62 or 63, wherein the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, IC-2, JIB-04, FH535, a docataxel/sulforaphane combination, pyrvinium pamoate, SKL2001, or a combination of two or more thereof.

Embodiment 65. The pharmaceutical composition of Embodiment 64, wherein the Wnt/β-catenin pathway inhibitor is tegatrabetan.

Embodiment 66. The pharmaceutical composition of any one of Embodiments 58 to 65, wherein the KRAS pathway inhibitor is a KRAS G12 inhibitor, a KRAS G13 inhibitor, or a combination thereof.

Embodiment 67. The pharmaceutical composition of any one of Embodiments 58 to 65, wherein the KRAS pathway inhibitor is a KRAS G12C inhibitor.

Embodiment 68. The pharmaceutical composition of any one of Embodiments 58 to 65, wherein the KRAS pathway inhibitor is a pan-KRAS inhibitor.

Embodiment 69. The pharmaceutical composition of any one of Embodiments 58 to 65, wherein the KRAS pathway inhibitor is sotorasib.

Embodiment 70. The pharmaceutical composition of any one of Embodiments 58 to 65, wherein the KRAS pathway inhibitor is adagrasib.

Embodiment 71. The pharmaceutical composition of any one of Embodiments 58 to 65, wherein the KRAS pathway inhibitor is BI-1701963 or N-((R)-1-(3-amino-5-(trifluoromethyl)phenyl)ethyl)-7-methoxy-2-methyl-6-(((S)-tetrahydrofuran-3-yl)oxy)quinazolin-4-amine.

Embodiment 72. The pharmaceutical composition of any one of Embodiments 58 to 65, wherein the KRAS pathway inhibitor is sotorasib, adagrasib, MRTX1133, ARS1620, BI-1701963, N-((R)-1-(3-amino-5-(trifluoromethyl)phenyl)ethyl)-7-methoxy-2-methyl-6-(((S)-tetrahydrofuran-3-yl)oxy)-quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl(2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one.

### EXAMPLES

The following examples are for purposes of illustration only and are not intended to limit the spirit or scope of the disclosure or claims.

ITGB4 and paxillin (PXN) are components of the focal adhesion complex, playing an important role in cisplatin resistance in KRAS mutant NSCLC. (Ref 32). These proteins are overexpressed in NSCLC and perturbing the ITGB4/PXN axis attenuated proliferation of NSCLC cells including KRAS mutant NSCLC cells by inducing mitochondrial stress, increased production of reactive oxygen species, and by suppressing DNA repair pathways, highlighting the novel, and potentially targetable, relationship between ITGB4 and PXN overexpression and KRAS mutation. Furthermore, we identified carfilzomib as a highly potent compound to sensitize cisplatin refractory KRAS mutant NSCLC cells, by disrupting the ITGB4/PXN axis highlighting the involvement of a non-genetic mechanism underlying cisplatin resistance in NSCLC. (Ref 54).

The ITGB4/PXN axis also plays an important role in the emergence of sotorasib resistance. Chronic sotorasib treatment can upregulate expression of WNT2 causing activation of WNT/β-catenin signaling as an additional mechanism of resistance. Further, we show that carfilzomib also acts synergistically with sotorasib and adagrasib to alleviate drug resistance in KRAS mutant NSCLC in a non-genetic fashion. Furthermore, employing MDS and mass spectrometry, we demonstrate how differences in conformational dynamics of the G12C mutant KRAS molecule bound to the 2 drugs account for the differences in efficacies and emergence of drug resistance via non-genetic mechanisms.

### Example 1

The inventors identified components of the focal adhesion complex, specifically integrin β4 (ITGB4) and paxillin (PXN) as being directly involved in KRAS-mutated non-small cell lung cancer (NSCLC) and modulated response to cisplatin. The inventors showed that both ITGB4 and PXN are overexpressed in NSCLC and perturbing the ITGB4/PXN axis is inversely correlated with KRAS signaling in KRAS-mutated NSCLC cell lines. This new finding highlights the novel relationship between ITGB4/PXN overexpression and KRAS mutation.

In addition, employing a virtual screen of an FDA-approved library of 1440 compounds was performed, the inventors identified the proteasome inhibitor carfilzomib as a highly potent (IC50, 0.8 nM) compound to sensitize cisplatin-refractory KRAS mutant NSCLC cells. Furthermore, treating NSCLC cell lines that express high levels of ITGB4 and PXN (H358 and H2009) were treated with carfilzomib, H358 cells carrying the KRAS G12C mutation were more sensitive than H2009 cells harboring the G12A mutation. RNAseq data indicates that carfilzomib inhibition of ITGB4 and PXN downstream signaling induces the expression of RAS-associated genes to overcome carfilzomib toxicity. Taken together, these observations indicate that treating patients with carfilzomib in addition to targeting mutant KRAS will be more efficacious than carfilzomib treatment alone.

Here, the inventors discovered that carfilzomib alleviates resistance to KRAS inhibitors. More specifically, the inventors show that the carfilzomib and sotorasib or adagrasib combination is a more effective treatment strategy than sotorasib or adagrasib alone in KRAS G12C mutant NSCLC cell lines, and carfilzomib can be used to overcome sotorasib or adagrasib resistance. Given that KRAS G12C is one of the most frequently mutated driver oncogene in several cancers in addition to NSCLC, these results demonstrate the ability to attenuate resistance to KRAS G12C-specific inhibitors in multiple cancer types.

Background: KRAS mutation is the most common gain-of-function alteration, accounting for about 30% of lung adenocarcinomas in western countries. Although multiple point mutations exist within KRAS in non-small cell lung cancer (NSCLC), the majority affect codon 12. Functionally, these point mutations result in amino acid substitutions that impair the GTPase activity of KRAS and render the oncoprotein constitutively active. These KRAS mutations impact patient prognosis and are negatively associated with response to targeted therapy and chemotherapy. Currently, at least 2 KRAS inhibitors, sotorasib and adagrasib that specifically inhibit KRAS G12C, are in clinical trials and look very promising. However, as with most drugs, it is likely that patients will develop resistance upon treatment.

Methods: To test this possibility, the inventors used the H23 and H358 non-small cell lung cancer (NSCLC) cell lines which are heterozygous for the KRAS mutation and the SW1573 cell line which is homozygous for the mutation. Gene expression difference were discerned employing RNAseq and the results were confirmed at the RNA level by qPCR and at the protein level by immunoblotting.

Results: The inventors observed the H358 (IC50 0.8 μM) and H23 (IC50 3.2 μM) were more sensitive to sotorasib whereas, SW1573 cells were relatively resistant (IC50 8 μM). However, upon continuous treatment, H23 cells developed resistance to sotorasib. RNAseq analysis of H23 cell lines pre- and post-treatment with sotorasib for a period of 7 weeks, identified MYC and E2F Target Hall Mark Pathways. Further, using qPCR assay the inventors validated the RNAseq data and observed more than 40 fold induction in expression of various genes such as WNT2, CFTR, and CCL2. Consistent with the qPCR data, the inventors also observed an increase in the protein expression of integrin beta 4 (ITGB4), WNT2 and activation of β-catenin upon treatment with sotorasib.

On the other hand, H358 cells which had higher expression of ITGB4, showed decreased expression upon sotorasib treatment, indicating that upregulation of ITGB4 is a mechanism of drug resistance to sotorasib treatment. Consistent with this finding, the inventors previous study revealed that ITGB4 knockdown sensitizes cells to cisplatin. The ITGB4 knockdown effect can also be recreated with protease inhibitor carfilzomib, which disrupts crucial interaction between PXN, FAK and ITGB4 and thus, inhibit focal adhesion complex-mediated survival signaling.

In addition, to the aforementioned molecules, the inventors observed an increase in the phosphorylation of AKT at serine 473 that further activates the survival signaling mediated through mTOR. AKT and mTOR activation inhibited GSK3B by which β-catenin degradation was blocked supporting WNT signaling. Thus, NSCLC cancer cells try to activate various signaling pathways which can eventually induce drug resistance upon sotorasib treatment. Finally, the inventors tested the efficacy of carfilzomib in sensitizing sotorasib resistant cell line SW1573. A combination of 8 μM or 16 μM of sotorasib and 9 nM of carfilzomib showed a synergistic effect in these cells. Immunoblotting experiments performed on these cell lysates post combination treatment showed reversal of the resistance inducing signaling like degradation of β-catenin, WNT2 and dephosphorylation of AKT, p70S6K and ERK.

Discussion: Taken together, the data unexpectedly demonstrates that the carfilzomib and sotorasib or adagrasib combination is a more effective treatment strategy than sotorasib or adagrasib alone in KRAS G12C mutant NSCLC cell lines, and carfilzomib can be used to overcome sotorasib or adagrasib resistance. Given that KRAS G12C is one of the most frequently mutated driver oncogene in several cancers in addition to NSCLC, the results will attenuate resistance to KRAS G12C-specific inhibitors in multiple cancer types.

Homozygous KRAS mutant cells are resistant to G12C inhibitors.

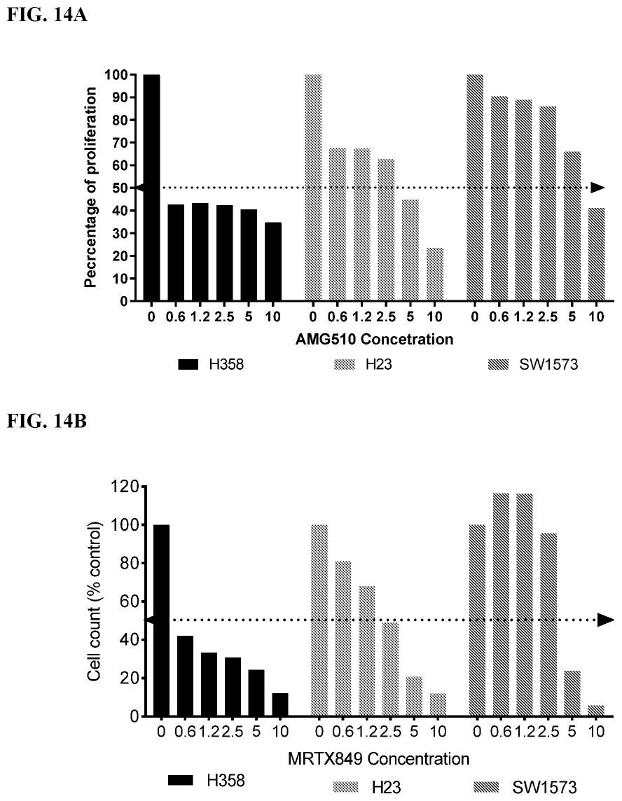

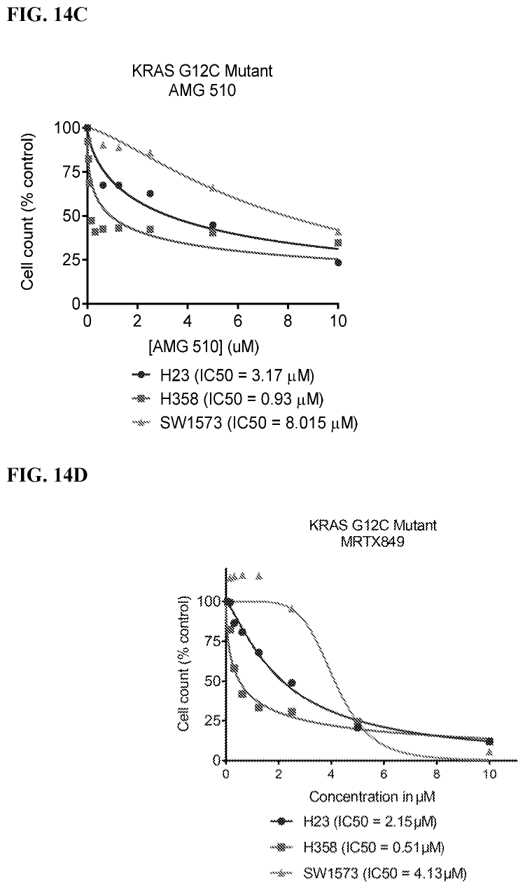

The inventors used the cell proliferation assay to determine the inhibitory effect of sotorasib or adagrasib on the LUAD cell lines H358, H23 or SW1573 (FIGS. 14A-14B). The inventors observed the H358 (IC50 0.8 μM) and H23 (IC50 3.2 μM) were more sensitive to sotorasib whereas, SW1573 cells were relatively resistant (IC50 8 μM) (FIG. 14C). The similar assay done with adagrasib we identified that H358 (IC50 0.31 μM), H23 (IC50 2.13 μM) and SW1573 (IC50 −4.13 μM) (FIG. 14D). The SW1573 is tolerant among the 3 cell lines for both drugs. However, adagrasib is more effective compared to sotorasib in sensitizing the resistant cell line SW1573.

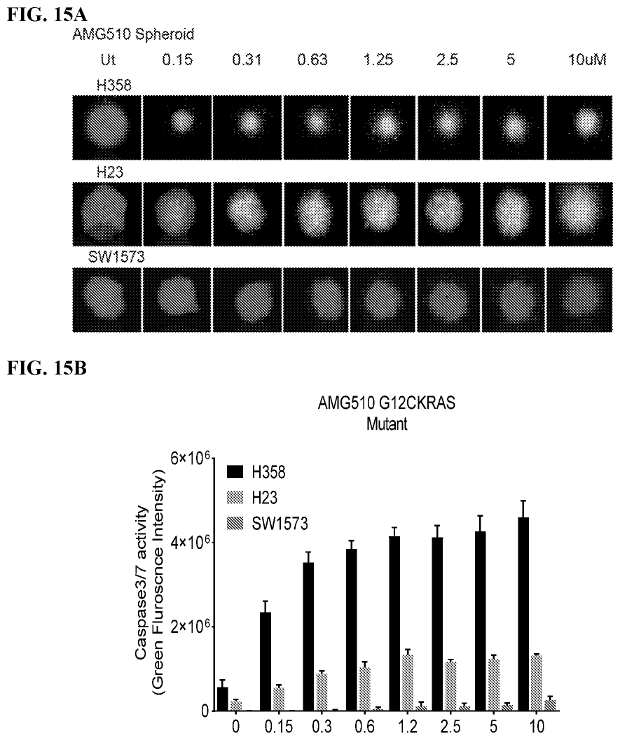

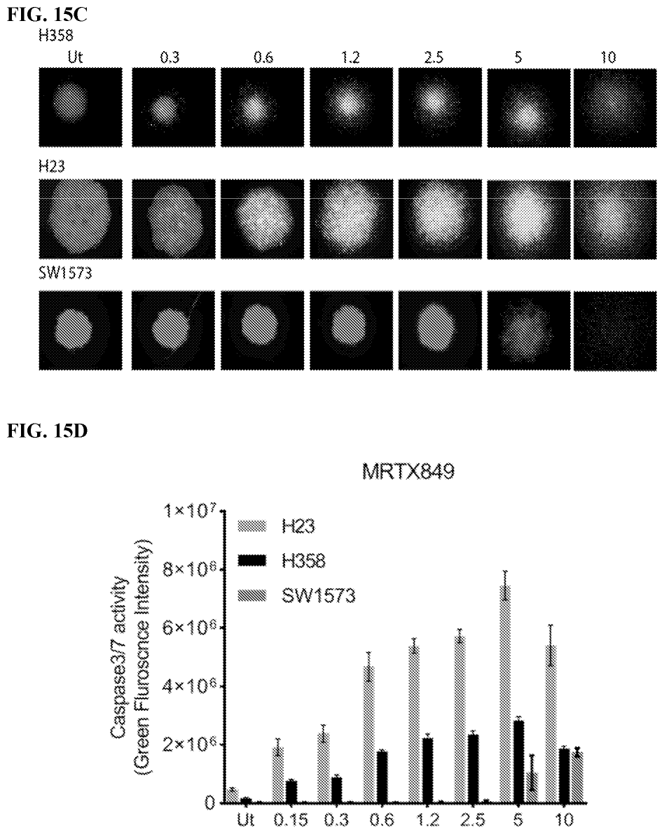

The inventors used the 3D spheroid assay to determine the effect of sotorasib and adagrasib in an in-vivo mimic condition. The results indicate that H358 cell line were most sensitive to the drugs and SW-1573 cell line are comparatively resistant to both the drug (FIGS. 15A-15D). The activation of apoptosis is marked with the increase in green fluorescence intensity. The green fluorescence intensity was represented as the bar graph and clearly show significant increase of apoptosis in H23 and H358 cells. The IC50 of both drugs in the 3 different cell line derived spheroid is shown in Table 1. Interestingly among the three cell lines the SW1573 is homozygous for the KRAS mutation.

TABLE 1

| | SW1573 | H23 | H358 |
|---|---|---|---|
| sotorasib | 12.62 μM | 12.34 μM | 0.0022 μM |
| adagrasib | 8.529 μM | 2.17 μM | 0.4163 μM |

AKT activation in response to sotorasib and adagrasib treatment.

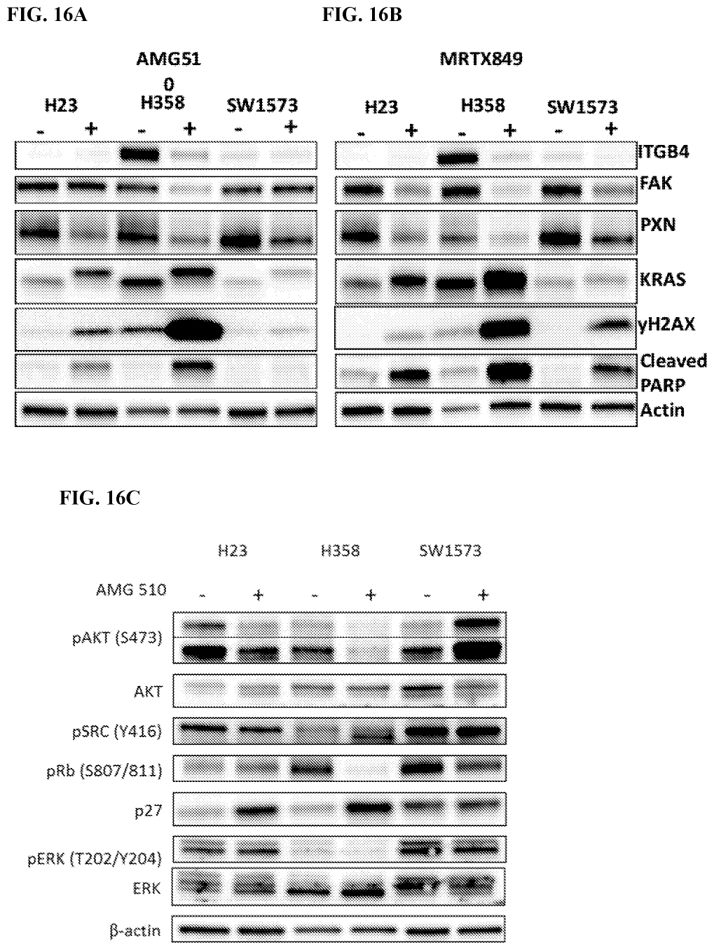

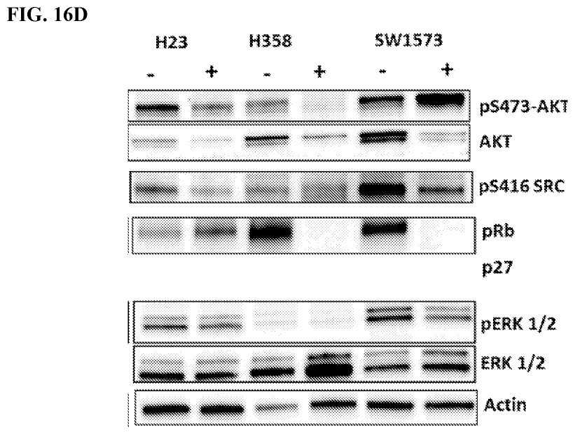

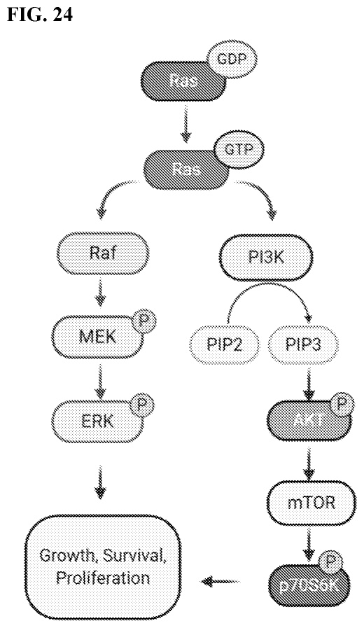

The inventors looked for the changes in the signaling in response to KRAS inhibition in the cell lines. Active KRAS can induce the activation of AKT or ERK signaling and this signaling need to be suppressed for inhibiting cancer cell growth (FIG. 24). The immunoblotting done on the lysate made after 72 hrs of drug treatment show no major change in phospho-ERK but an activation was observed in the phosphorylation of AKT. AKT activation leads to mTOR activation and cell survival (FIGS. 16A-16B). Analysis of the focal adhesion associated molecules indicate down regulation of ITGB4, PXN and FAK in response to drug treatment (FIGS. 16C-16D). H358 cells which had highest expression of ITGB4, showed decreased protein levels, indicating previous that ITGB4 dependent cells are sensitive to reduce expression of ITGB4.

Upregulation of WNT2, CFTR and CCL2 Genes in Isogenic Sotorasib Resistant Cell Line.

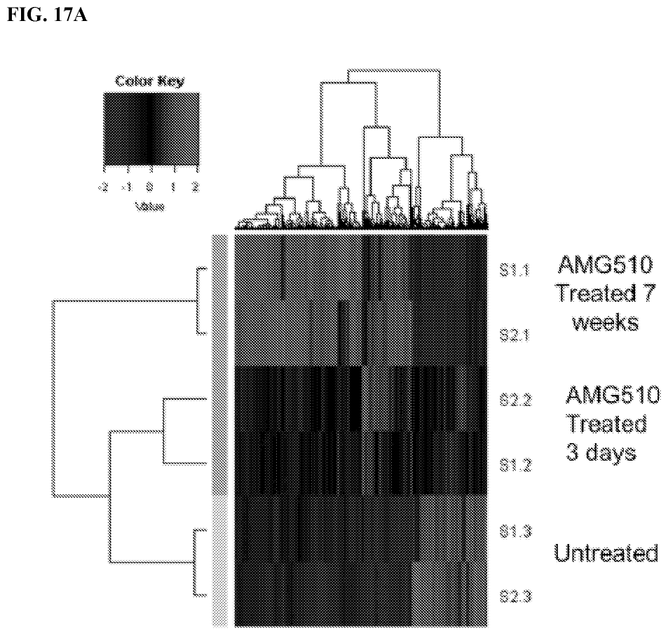

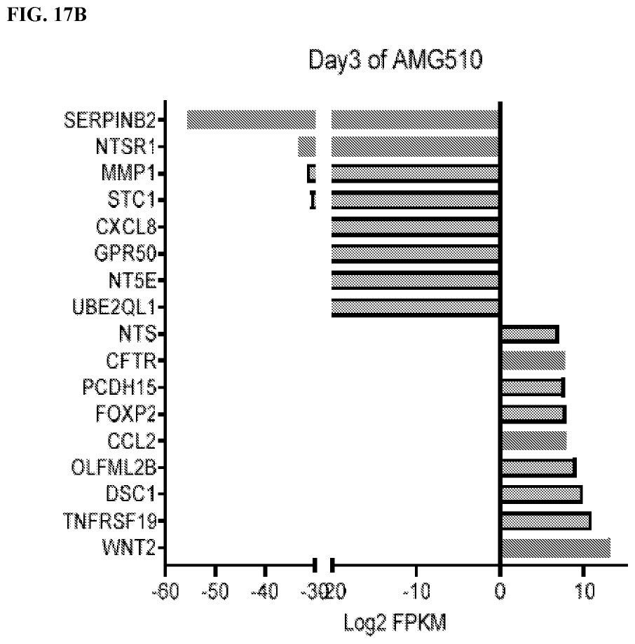

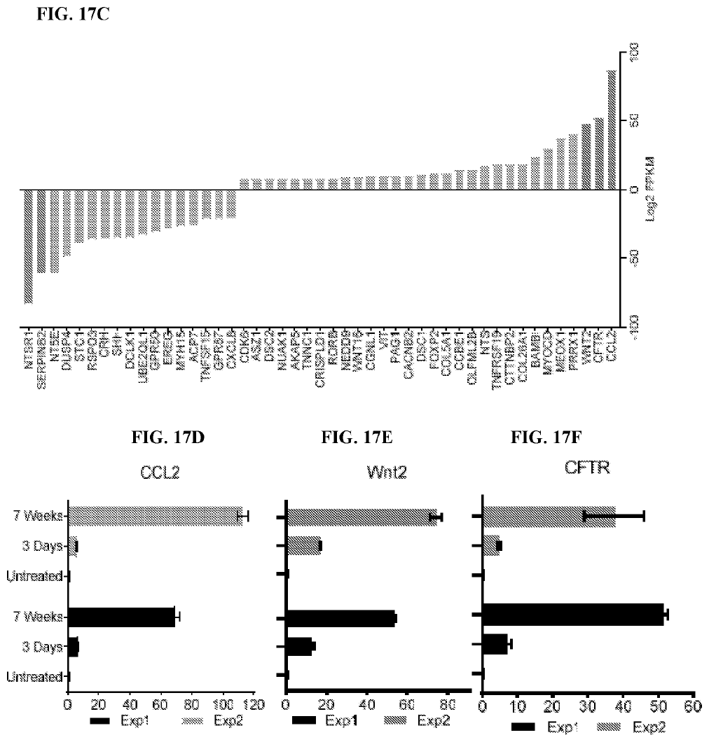

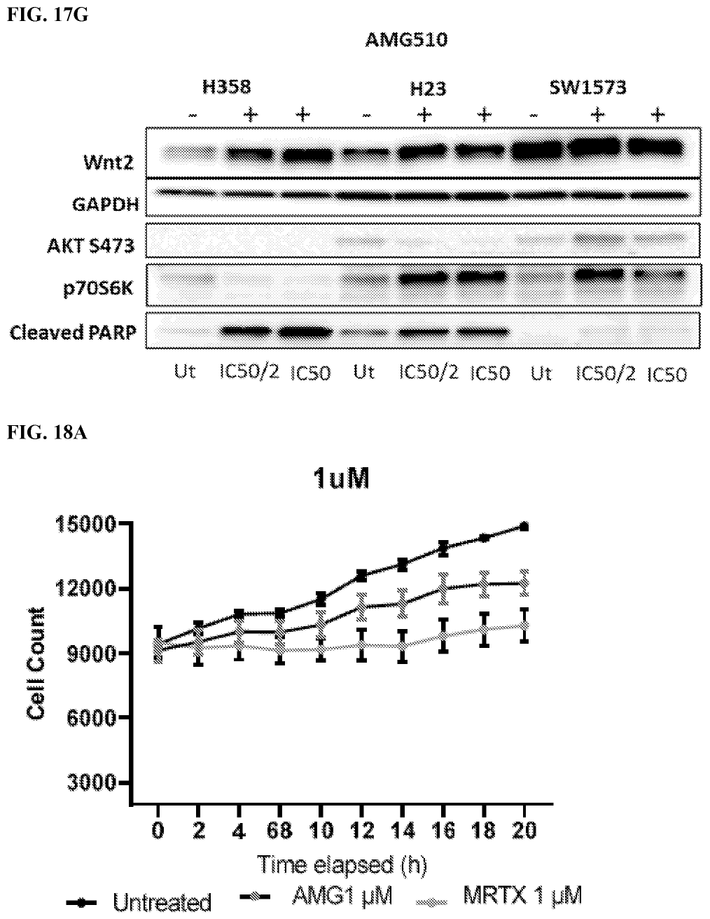

To understand the mechanism of drug resistance the inventors used H23 cell line and treat it for a period of seven weeks with the drug sotorasib. The drug treatment what initiated at dose of 1 μM which was finally raised up to 7.5 μM. RNA was extracted from the sotorasib resistant cell line and sent for RNAseq analysis in comparison to the control and the cells which were treated with IC50 dose for the drug for 3 days. There are various genes which get upregulated and down regulated in response to sotorasib treatment as shown in heatmap and Venn diagram (FIG. 17A). RNAseq analysis revealed that WNT2, CFTR or CCl2 are the genes which were constantly upregulated in the H23 cell line in 3 days as well as in the continuous treatment (FIGS. 17B-17F). Further, using qPCR assay we validated the RNAseq data and observed more than 40 fold induction in expression of various genes such as WNT2, CFTR and CCl2. (FIGS. 17D-17F). Further the analysis also identified activation of MYC and E2F Target Hall Mark Pathways which may help H23 cells in withstanding drug toxicity (data not shown). The immunoblotting done on the lysates treated with half does of IC50 or IC50 dose showed increased expression of WNT2, phosphorylation of AKT and p70 S6K (FIG. 17G).

Activation of β-Catenin and ITGB4 Protein in Response to Sotorasib Treatment.

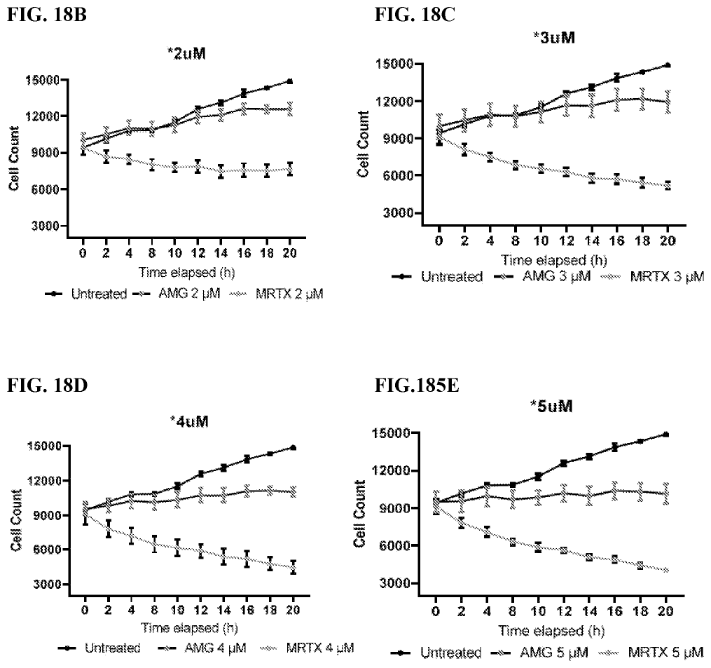

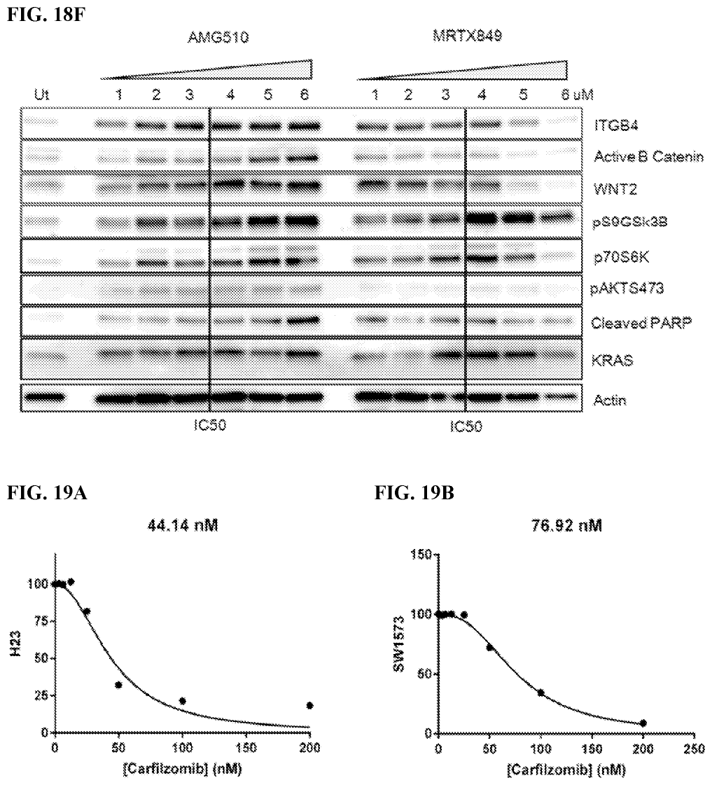

The H23 cell line was treated with increasing doses of sotorasib or adagrasib. Live cell imaging and analysis was done to measure the cell proliferation for all the doses for 24 hr, to find that adagrasib effectively inhibited cell proliferation even at doses lesser than IC50 dose of about 3 μM compared to sotorasib treatment (FIGS. 18A-18E). Lysates were made after 72 hr drug treatment and immunoblotting was done on the lysates. ITGB4, WNT2 and β-catenin expression increased in response to drug treatment. Similarly, the AKT and 70Sk kinase phosphorylation also increased in response to increasing concentration of drug. On the other hand, with adagrasib all these signaling changes reversed at drug concentration of 5 μM and 6 μM. This explains the difference in the behavior of cells in response to sotorasib or adagrasib (FIG. 18F).

Carfilzomib Sensitizes Sotorasib Resistant Cell Lines.

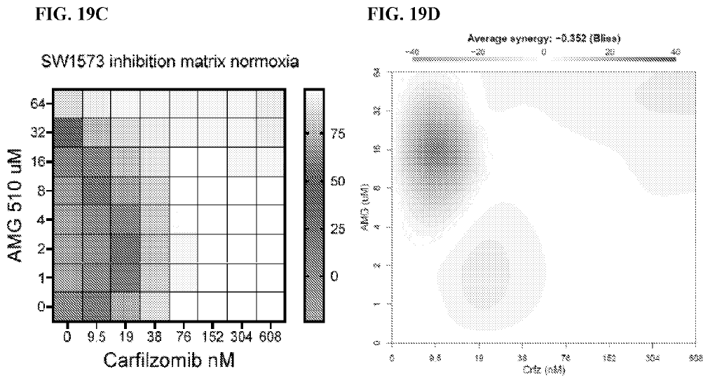

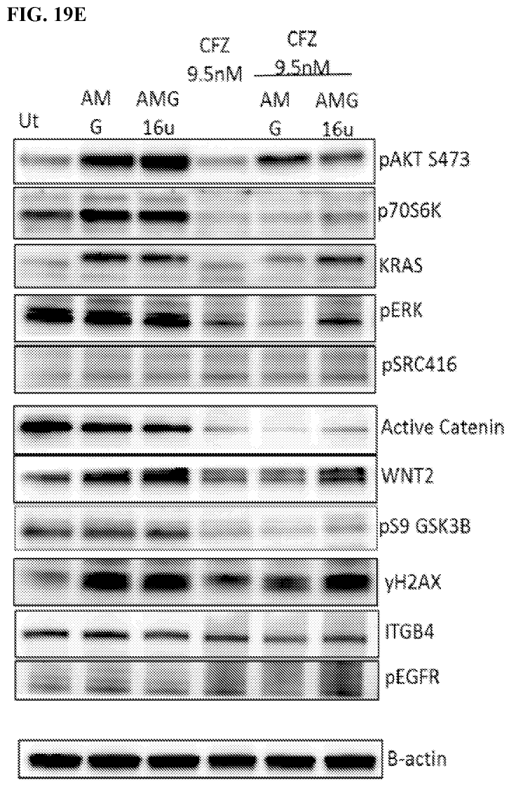

Upregulation of ITGB4 is a mechanism of drug resistance to sotorasib treatment. Consistently, the inventors previous study revealed that ITGB4 knockdown sensitizes cells to cisplatin. The ITGB4 knockdown effect can also be recreated with protease inhibitor carfilzomib, which disrupts crucial interaction between PXN, FAK and ITGB4 and thus, inhibit focal adhesion complex-mediated survival signaling. First the inventors determined the IC50 dose of carfilzomib in the H23, H358 and SW1573 cell lines (FIGS. 19A-19B). Then we tested the efficacy of carfilzomib in sensitizing sotorasib resistant cell line SW1573. A combination of 8 μM or 16 μM of sotorasib and 9 nM of carfilzomib showed a synergistic effect in these cells as measured by the Bliss synergy analysis (FIGS. 19C-19D). Immunoblotting experiments performed on these cell lysates post combination treatment, showed reversal of the resistance inducing signaling like degradation of β-catenin, WNT2 and dephosphorylation of AKT, p70S6K and ERK (FIG. 19E).

### Example 2

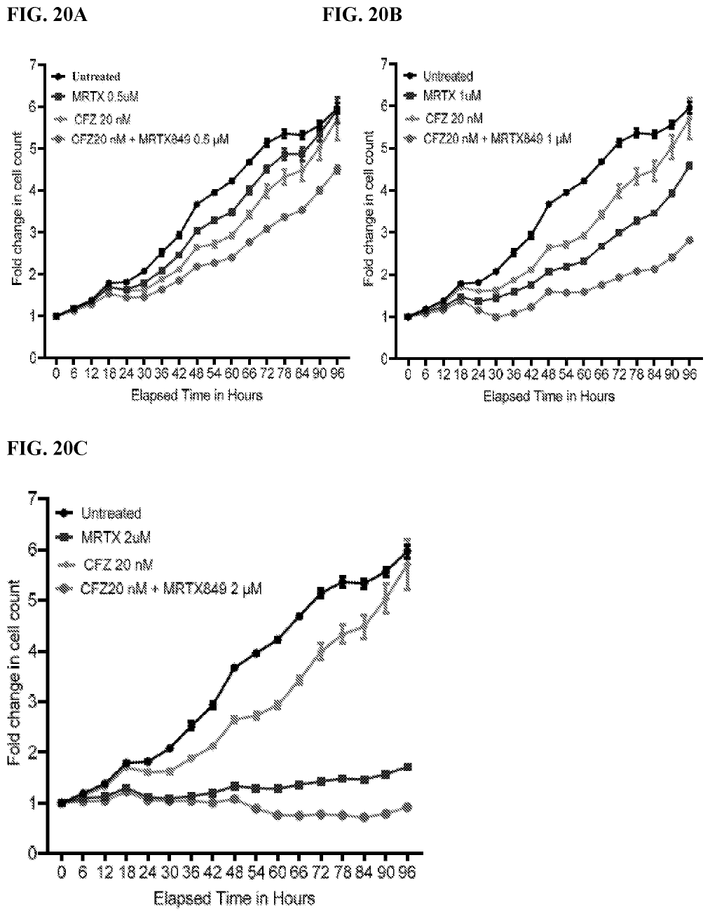

Drug Combination Experiment: The SW1573 cell lines expressing mKAte2 were selected for testing the effect of adagrasib and carfilzomib drug combination. The cells were seeded at density of 10000 cells/well 12 hours prior to the experiment. Once the cells attached to the plate the media was removed and the drugs were added to the cells. In the experiment, a sub-lethal concentration of carfilzomib was used and kept constant throughout the experiment whereas the concentration of adagrasib used was variable ranging from 0.5 μM, 1 μM or 2 μM. Cell proliferation was observed using the IncuCyte Live Imaging System for 72 hrs. Drug combination assays were done to determine the combination effect of Carfilzomib and adagrasib on the cell proliferation. FIGS. 20A-20C show proliferation in the absence of drug, adagrasib alone (0.5 μM, 1 μM, or 2 μM), carfilzomib alone at 20 nM), and the combination of adagrasib and carfilzomib. adagrasib at 0.5 μM did not inhibit proliferation (FIG. 20A) whereas the combination has an additive effect (FIG. 20C). adagrasib at 1 μM effectively inhibited proliferation by 25% and the combination inhibited proliferation by 53%, such that the effect of carfilzomib and 1 μM adagrasib was synergistic (FIG. 20B). The data indicates that adagrasib and carfilzomib show synergy at sub-lethal doses.

### Example 3

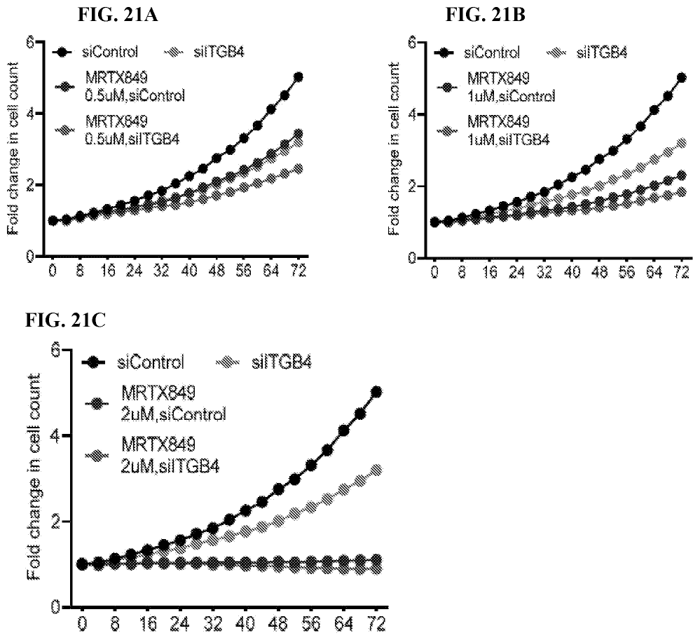

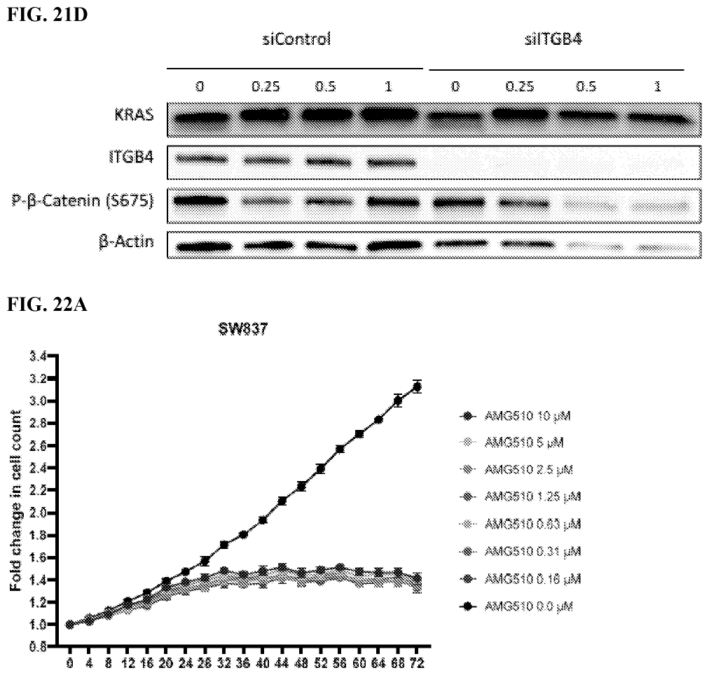

ITGB4 knockdown and adagrasib combination experiment. Knockdown of ITGB4 (Cat #SR302473C) at the mRNA level in the SW1573 cells was done using siRNAs purchased from OriGene Technologies (Rockville, Md., USA). JetPRIME transfection reagent (Polyplus Transfection, Illkirch, France) was used to transfect the siRNAs according to the manufacturer's protocol. Cells were seeded in 6-well plates (200,000 cells/well) and allowed to adhere overnight. Next day, 10 nM siRNA was transfected with 4 μl jetPRIME reagent in complete growth medium for each well. Next day cells were trypsinized, counted and re-plated on the 96 well plate for cell proliferation and drug assay. Once the cells attached to the plate, the G12C inhibitor adagrasib was added at three different concentrations 0.5, 1, and 2 μM. Cell growth medium was changed the next day and expression was detected 72 h post-transfection by immunoblotting. The proliferation without any drug, with adagrasib, with ITGB4 knockdown, and with the combination effect is shown in FIGS. 21A-21C. adagrasib at 0.5 μM inhibited proliferation by 38% and ITGB4 knockdown inhibited proliferation by 34% but the combination of knockdown and adagrasib reduced proliferation by 50%, indicating ITGB4 knockdown increased drug sensitivity. However, increasing the dose of adagrasib from 0.5 μM to 1 μM or 2 μM did not add much to drug inhibition. Immunoblotting done on the 72nd hour of drug treatment showed complete reduction in the expression of ITGB4. Further, in the ITGB4 knockdown cells, the activated β-catenin level reduced faster compared to control (FIG. 21D) which may explain their increased sensitivity.

### Example 4

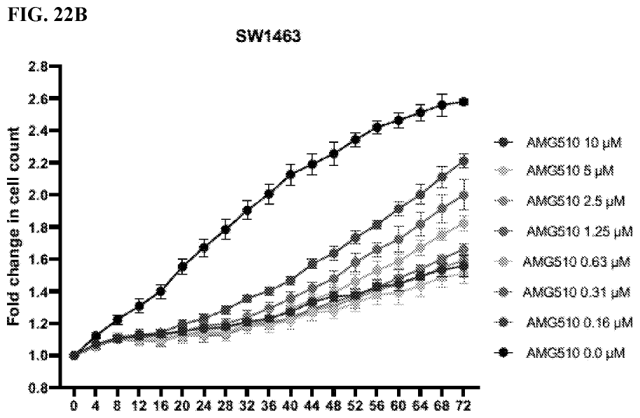

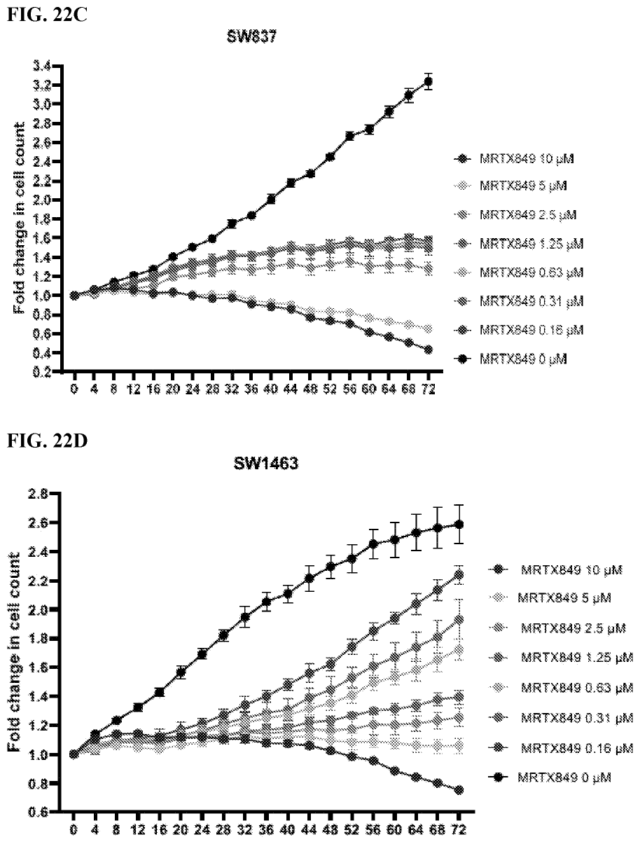

Adagrasib inhibited cell proliferation effectively compared to sotorasib. For this experiment the inventors chose SW837 and SW1463 colorectal cell line. The SW837 cell is heterozygous for the G12C mutation whereas the SW1463 is homozygous for the G12C mutation. A stable cell line expressing mKATE2 for Incucyte live cell imaging and analysis was generated. The proliferation of cells without any drug or the cells treated with adagrasib or sotorasib is shown in FIGS. 22A-22D. The proliferation mutation data indicates that heterozygous mutant SW837 cell line was highly sensitive to even the lowest dose (0.16 μM) of sotorasib and increasing the dose did not add much to the inhibition (FIG. 22A). In the SW1463, the 10 μM dose of sotorasib inhibited the proliferation by 40% only (FIG. 22B), again indicating that the homozygous mutants are more tolerant to sotorasib. Further, minimal drug concentration of adagrasib (0.16 μM) has a strong inhibitory cytostatic effect on the homozygous cell line and at a dose of 5 or 10 μM adagrasib induced cell death within 72 hours (FIG. 22C). In the SW1463, adagrasib had a dose dependent effect on the proliferation. Up to a dose of 5 μM, adagrasib has a cytostatic effect on the SW1463 and at a dose of 10 μM, the drug has cytotoxic effect (FIG. 22D).

### Example 5

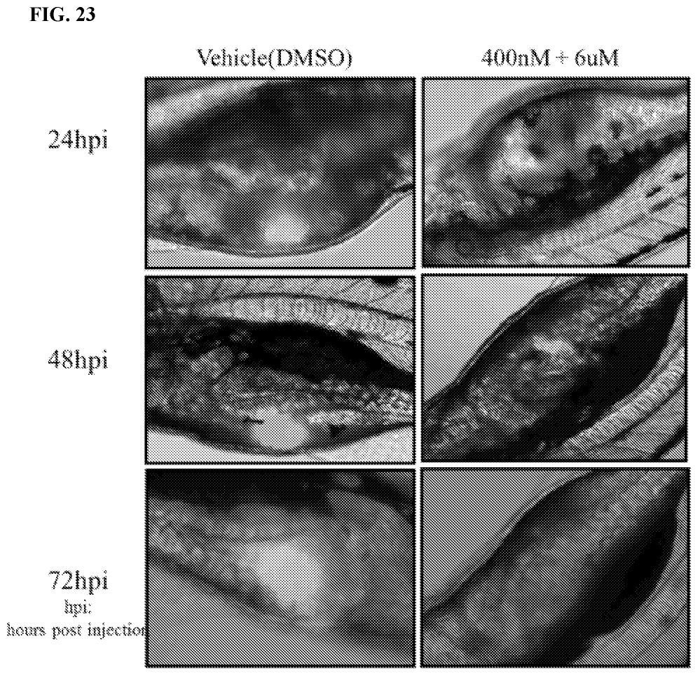

The inventors microinjected SW1573 cells into the perivitelline space of 48 hour post-fertilization (hpf) zebrafish larvae. Twenty-four hours following microinjection, the larvae were treated with the drug combination of sotorasib (Drug2) and Carfilzomib (Drug1) or individual doses of sotorasib or Carfilzomib. The experimental results indicate that the combination of sotorasib and Carfilzomib obliterated the SW1573 cells at combination of 6 μM and 400 nM, respectively (FIG. 23). The same effect was observed when 12 μM of sotorasib or 1.6 μM of Carfilzomib was used individually. The experiments conducted are shown in Table 2 below

TABLE 2

| Carfilzomib | Sotorasib | Combination of Carfilzomib and Sotorasib |
|---|---|---|
| 400 nM | 6 μM | 400 nM + 6 μM |
| 800 nM | 12 μM | 800 nM + 12 μM |
| 1.6 μM | 18 μM | 1.6 μM + 18 μM |

The most effective doses were 1.6 μM of carfilzomib alone; 12 μM of sotorasib alone; and the combination of 400 nM carfilzomib and 6 μM of sotorasib. The doses were toxic for 18 μM of sotorasib alone; for the combination of 800 nM carfilzomib and 12 μM of sotorasib; and for the combination of 1.6 μM carfilzomib and 18 μM of sotorasib.

These in vitro results demonstrate the drug combination is effective against G12C resistant cell lines.

### Example 6

Homozygous KRAS Mutant Cells are More Resistant to Sotorasib than Heterozygous Cells

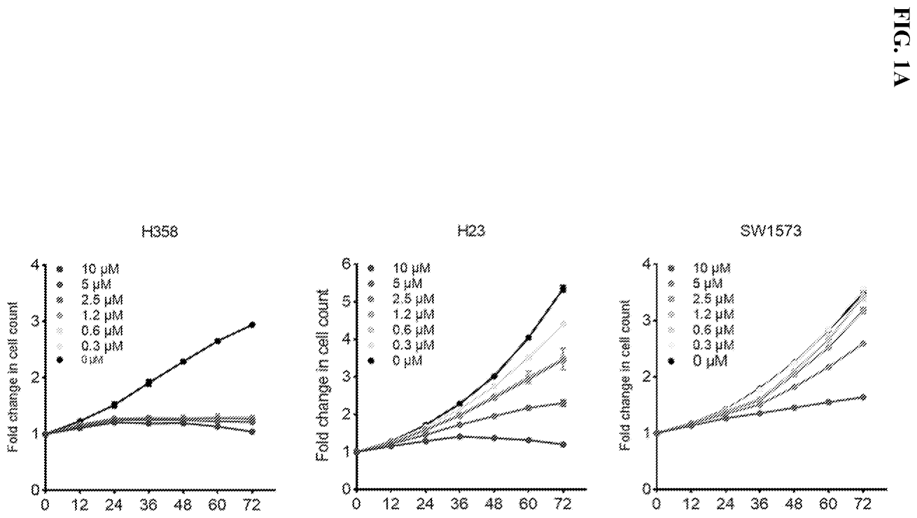

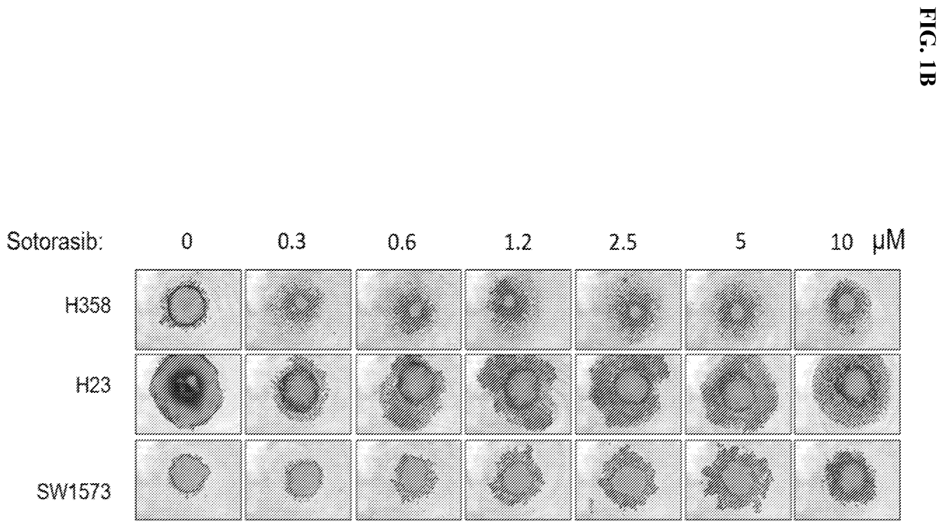

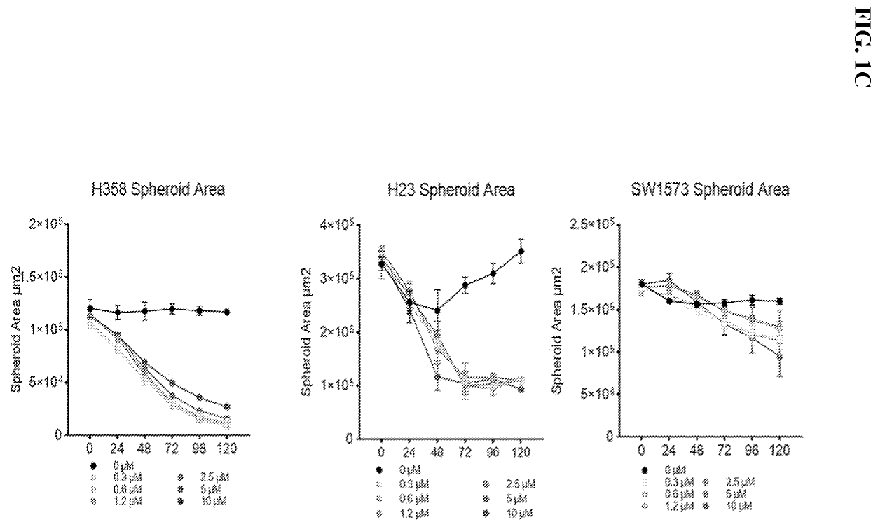

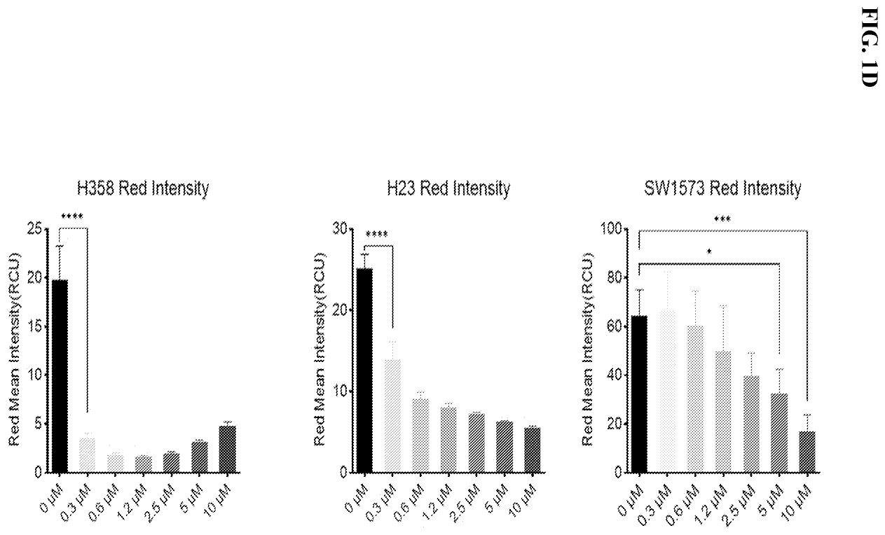

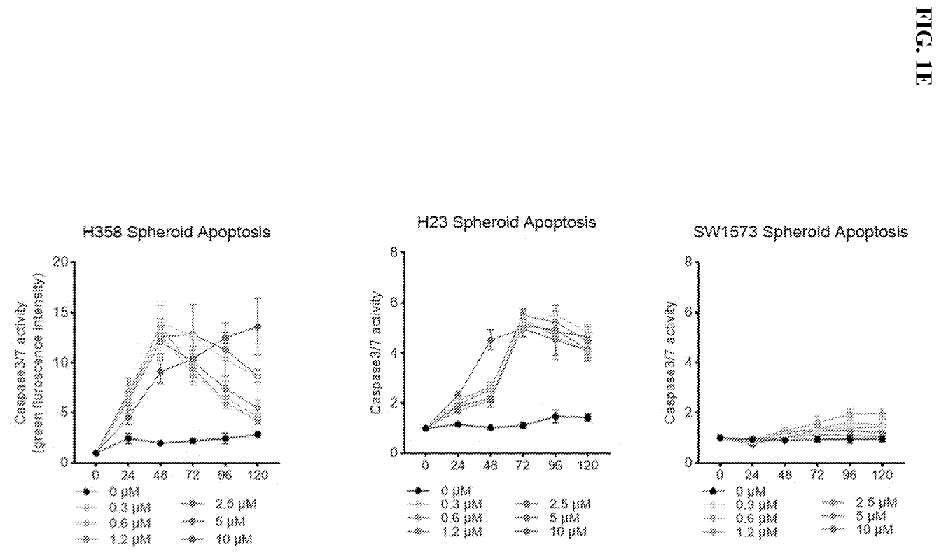

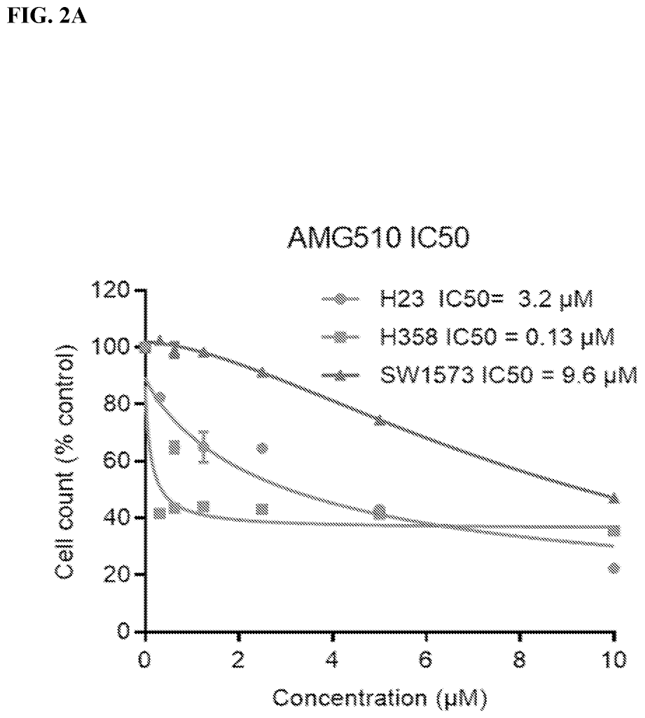

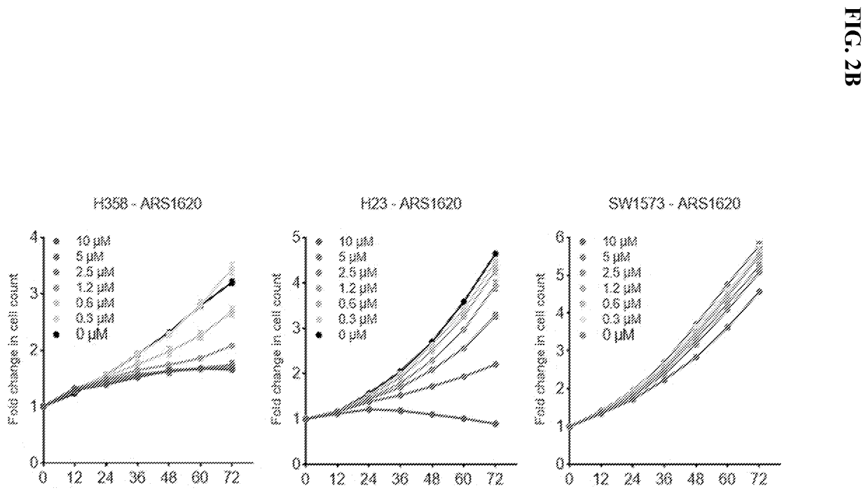

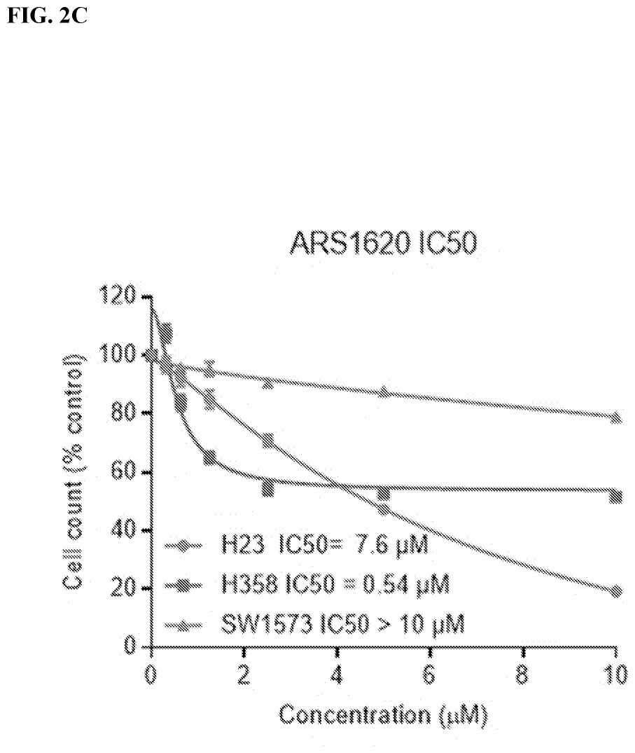

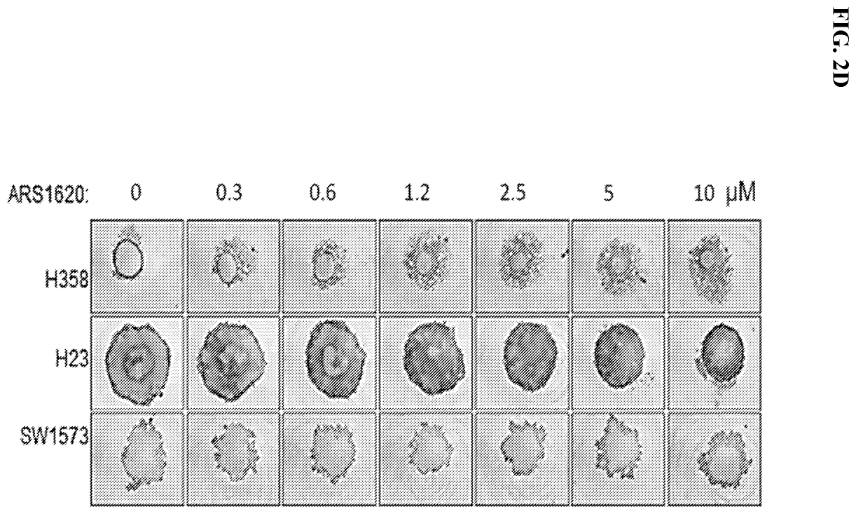

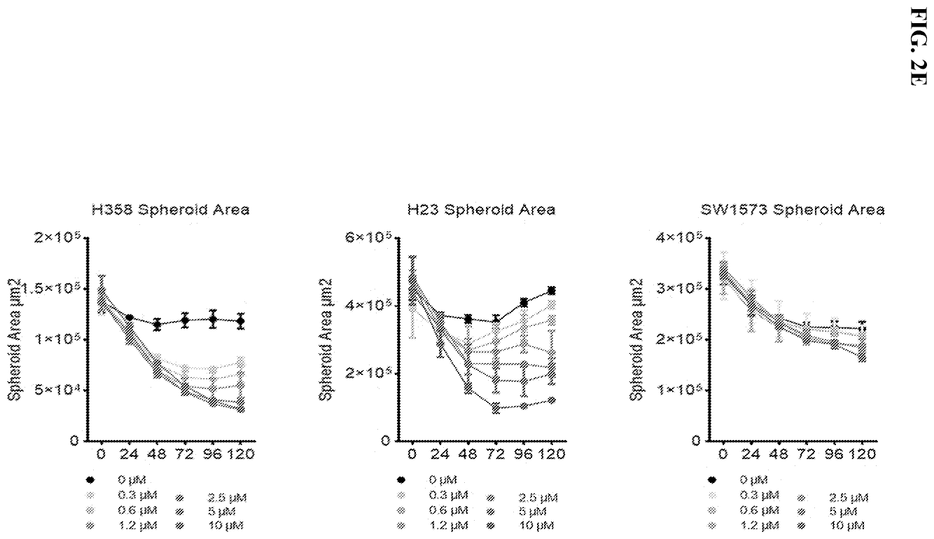

To determine the efficacy of sotorasib (drug), three KRAS G12C mutant LUAD cell lines were used that were either heterozygous (H358 and dH23) or homozygous (SW1573) with respect to the G12C locus. The cells grown in 2D cultures were treated with various concentrations of the drug, and the effect on cell proliferation was determined in real time using the live cell proliferation assay (IncuCyte, Essen Bioscience, Inc.) as described in the methods. Proliferation of H358 cells was inhibited by about 60% at the lowest concentration (0.61 μM) of the drug and increasing the concentration of the drug to 10 μM showed no significant increase in inhibition (left graph in FIG. 1A). In contrast, H23 cells showed a dose-dependent response and were inhibited by about 20% at 0.3 μM and by about 80% at 10 μM of sotorasib. The highest concentration of the drug had a cytotoxic effect on these cells (middle graph in FIG. 1A). A dose-dependent response was also seen in SW1573 cells with the highest dose (10 μM) inhibiting them by about 50% (right graph in FIG. 1A). IC50 values determined using the linear regression plot, showed that while the H358 cells were sensitive (IC50, 0.13 μM), the H23 cells had an intermediate sensitivity (IC50, 3.2 μM) and the SW1573 cells were resistant (IC50, 9.5 μM) (FIG. 2A). Furthermore, genomic sequencing revealed that while the SW1573 cells are homozygous with respect to the KRAS G12C mutation, the H358 and the H23 cells are heterozygous. These data are consistent with the cellosaurus cell line database of ExPasy.

To determine the sensitivities of the same cells in 3D, mKate expressing cells were used to generate spheroids. Spheroids were monitored in real time using the IncuCyte live cell imaging system (FIG. 1B). In the case of H358 cells, 0.3 μM of sotorasib inhibited spheroid area by 90%, but further increase in the drug concentration did not have any additional effect. Similarly, H23 spheroids were 70% inhibited at 0.3 μM of sotorasib and no further increase in inhibition was observed with increase in drug concentration. However, 0.3 μM of sotorasib had only 30% inhibitory effect on the spheroid area of SW1573 cells and further increase in concentration led to a marginal increase in its inhibitory effect (about 40% inhibition at 10 μM) (FIG. 1C). Next, the red intensity was measured as an additional readout and observed that at 0.3 μM of sotorasib, H358 cells had about 80% drop in the spheroid intensity compared to about 40% drop for the H23 cells and there was no change in the case of the SW1573 spheroids. However, the spheroid intensity dropped to 80% and 70% for H23 and SW1573, respectively, at concentration of 10 μM (FIG. 1D). It also determined the caspase activity and observed significant increase in H358 cells compared to H23 and SW1573 cells. Interestingly, the SW1573 spheroids did not show any increase in the caspase activity even though there was a drop spheroid area and red intensity, indicating the sotorasib suppresses SW1573 spheroid growth but does not induce cell death (FIG. 1E). Together, both the 2D and 3D data indicate that the homozygous KRAS mutant SW1573 cells are least responsive compared to the heterozygous H358 and H23 cells.

Testing of the efficacy of the covalent inhibitor ARS1620 was done on these three cell lines. ARS1620 at a concentration of 1.2 μM inhibited H358 cell growth by 40% in 72 hours, and increasing the concentration to 2.5, 5 or 10 μM induced 50% inhibition of cell growth (left graph in FIG. 2B). Similarly, H23 cell growth was inhibited by 30% at 1.2 of ARS1620 and inhibition increased to 50% and 80% with 5 and 10 μM, respectively. Further, in the case of the SW1573 cell line, ARS1620 inhibited 20% of cell growth at a maximum concentration of 10 μM (right graph in FIG. 2B). The IC50 values for the H358, H23 and SW1573 cell lines were 7.6 μM and greater than 10 μM, respectively (FIG. 2C). The inhibition assay was performed with ARS1620 on cell line derived spheroids (FIG. 2D). Spheroid area and growth kinetics data indicated a 70% reduction in spheroid growth at 10 μM of ARS1620 for both H358 and H23 cell lines, and about 25% reduction in the SW1573 cell line (FIG. 2E). Similarly, the analysis of mean red intensity showed 5 μM of ARS1620 reduced H358 spheroid red intensity by 80%, H23 spheroids by 50%, and the SW1573 spheroids by 40%, refer to FIG. 2F. Finally, the analysis of caspase3/7 activity showed a 19-fold increase in activity within 24 h of 2.5 μM of ARS1620 treatment for the H358 cells whereas there was only a 5-fold increase for the H23 cells, and no change in the SW1573 cells, refer to FIG. 2G. These data indicated that H358 cell line is highly sensitive to ARS1620 followed by H23, whereas the SW1573 cell line is the least sensitive to this covalent inhibitor.

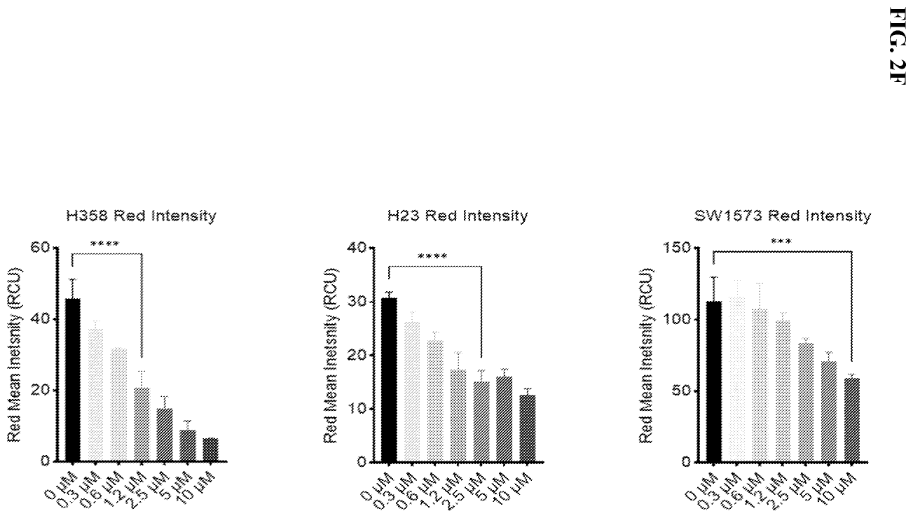

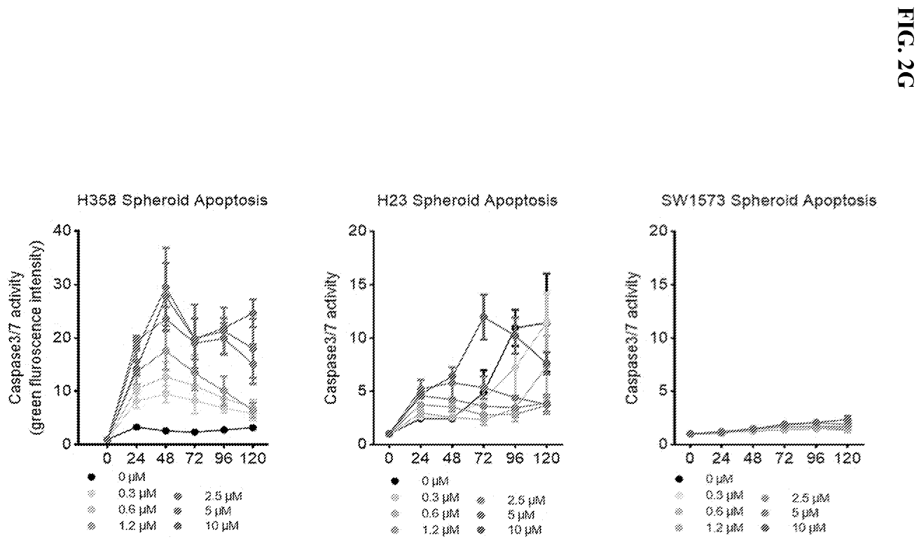

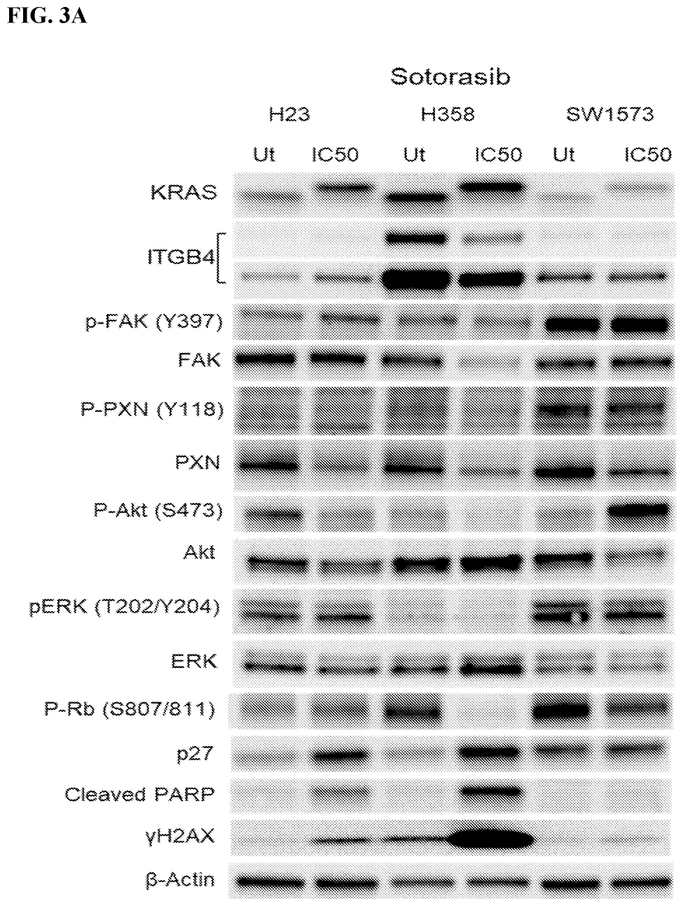

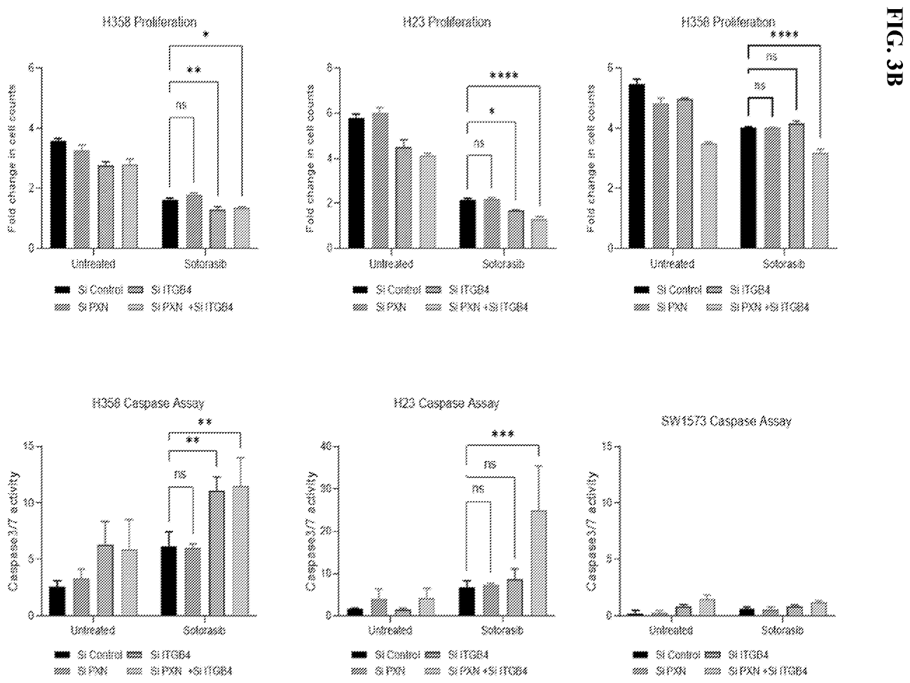

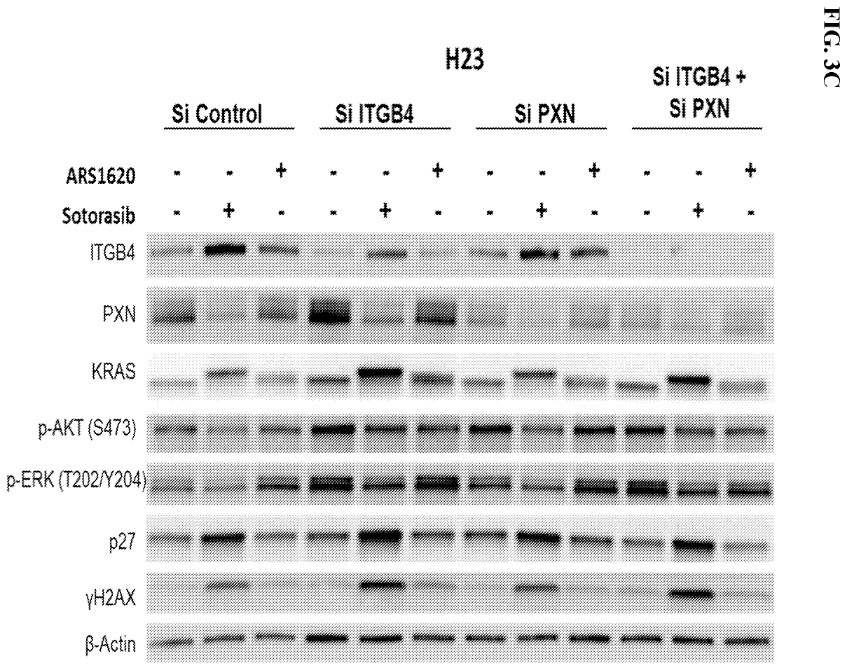

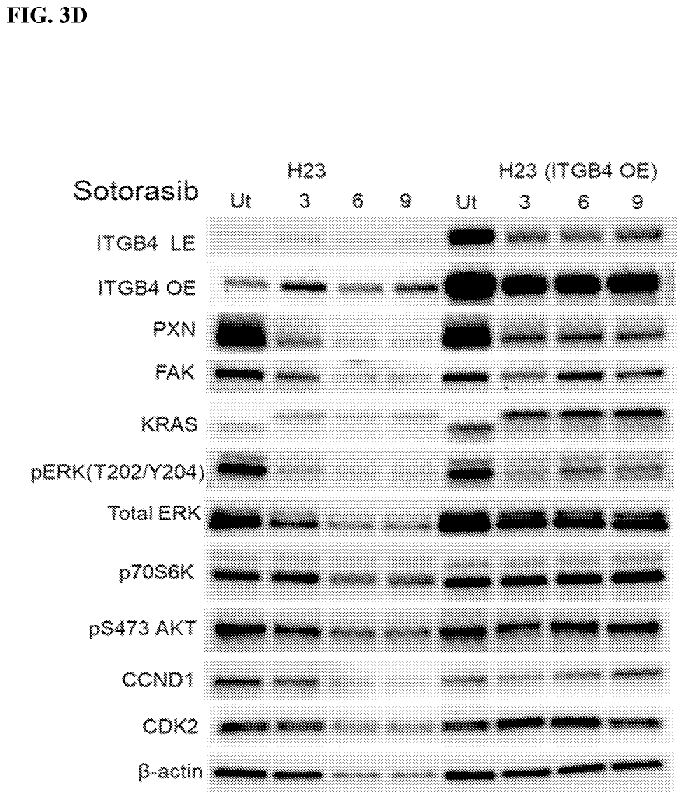

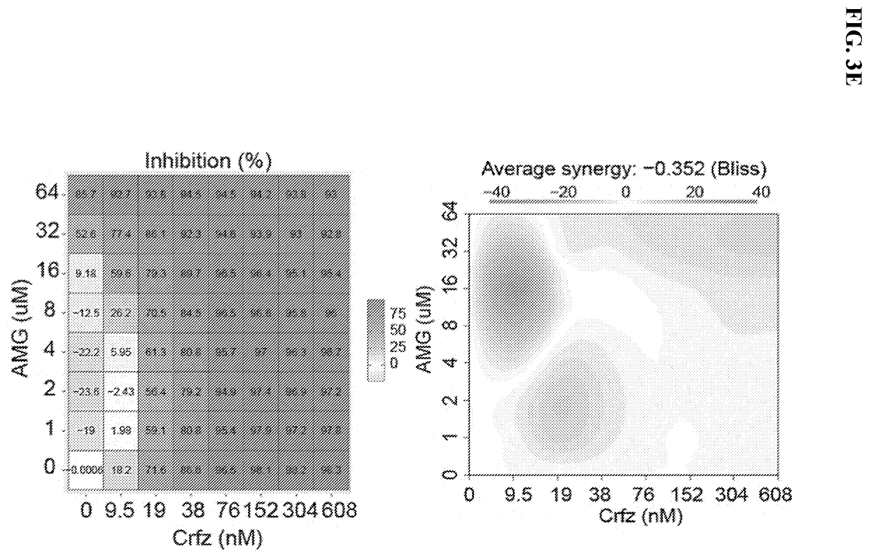

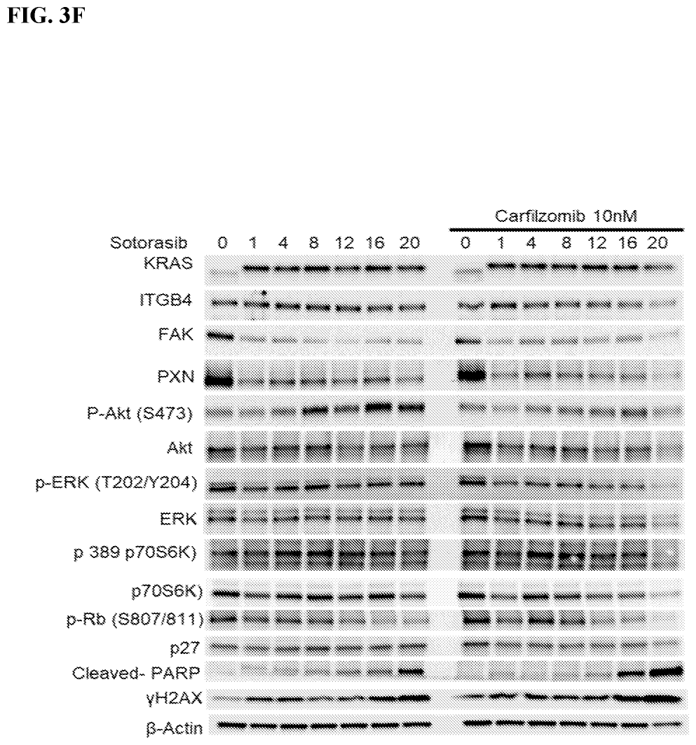

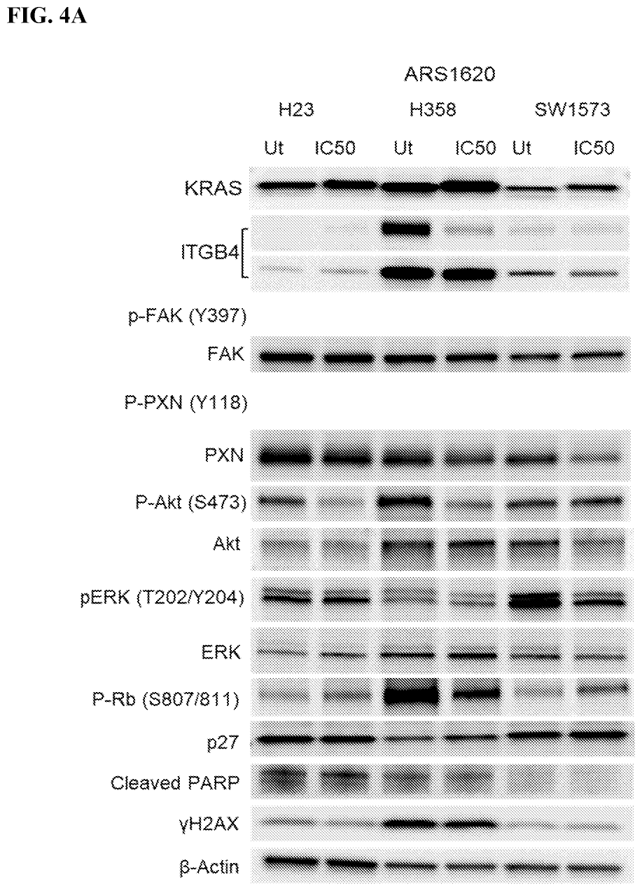

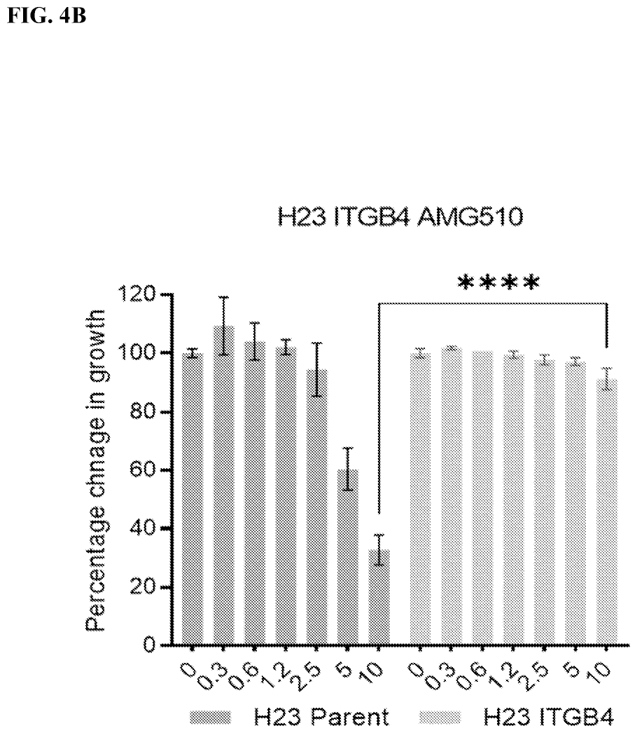

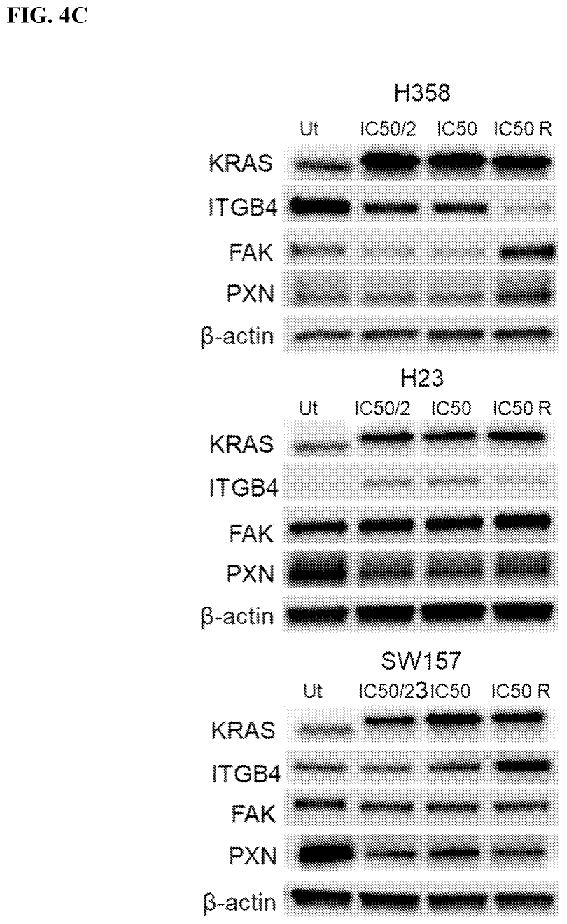

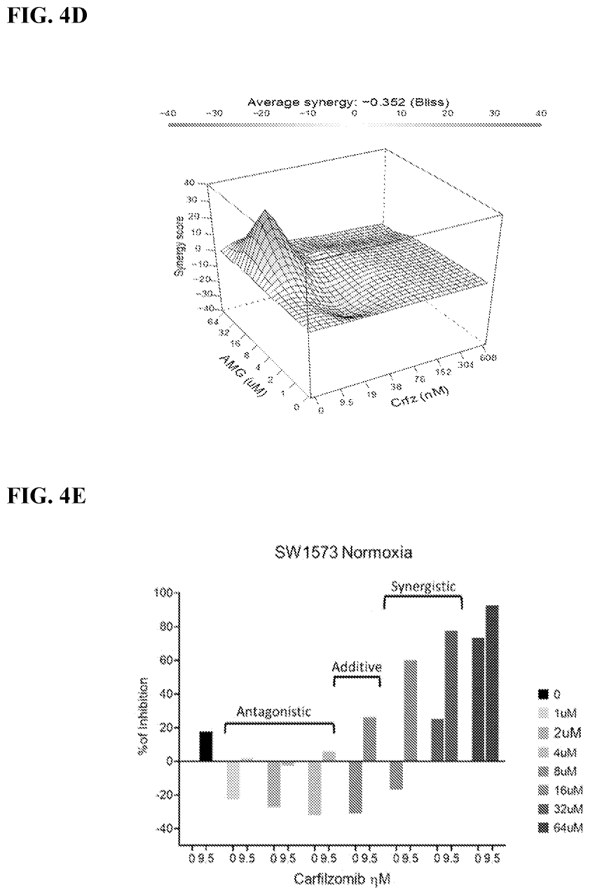

Sotorasib activates the AKT/mTOR signaling: To elucidate the downstream signaling events that ensure drug treatment, the cells were treated with sotorasib at the IC50 concentration that was determined for each cell line. Firstly, examination of the stability of ITGB4 was done. In H358 cells that were sensitive to sotorasib, ITGB4 was rapidly degraded. In addition, there was a significant activation of the apoptotic markers cleaved PARP, DNA damage marker γH2AX and cell cycle inhibitor p27, but a decrease in the expression of phospho-AKT and phospho-Rb as well as loss of phospho-ERK. Consistent with the cell proliferation data, it was observed that a moderate activation of cleaved PARP and γH2AX in H23 cells, whereas the SW1573 cells did not show any significant activation of these markers. Further, an activation of AKT as well as p70 S6 kinase, a downstream effector of AKT activation was observed, indicating that the AKT/mTOR pathway is activated in these cells. Interestingly, we observed a partial upregulation of ITGB4 in the H23 cells but not in the SW1573 cells, and a decrease in expression of PXN but no significant change in the levels of focal adhesion kinase (FAK) (FIG. 3A). Immunoblotting analysis of ARS1620 treated cells also showed decrease in expression of ITGB4 and a drop in AKT phosphorylation for the sensitive cell lines H358 and no major change for the H23 and SW1573 cell lines (FIG. 4A).

Knocking down ITGB4 and PXN sensitizes cells to sotorasib: To validate the role of ITGB4 and PXN in sotorasib resistance, ITGB4 or PXN or both TGB4 and PXN were knocked down in the 3 cell lines using siRNA (small interfering ribonucleic acid). Twenty-four hours after transfection, cells were split into 2 groups. The first group was untreated, and the 2nd group was treated with IC50 concentration of sotorasib. Cell proliferation and cell death were measured in real time as described above. In the untreated condition, partial inhibition of proliferation was observed for the 3 cell lines upon knocking down both ITGB4 and PXN (FIG. 3B). However, in the presence of sotorasib, the effect on inhibition was significant for ITGB4 knockdown or ITGB4/PXN double knockdown cells but not PXN. Further, there was a significant activation of caspase-3/7 in presence of sotorasib in the H358 and H23 cells upon knocking down both ITGB4 and PXN. On the contrary, the double knockdown did not induce any caspase activity for the sotorasib resistant cell line SW1573, which suggests there are alternative mechanisms of resistance in this cell line (FIG. 3B). Next, ITGB4 or PXN or both were knocked down and the cells were treated with sotorasib at the IC50 concentration to determine perturbations in the AKT/mTOR signaling pathway. In the presence of sotorasib, phosphorylation of AKT and p70 S6 kinase was abrogated by knocking down ITGB4 suggesting that increase in ITGB4 expression is crucial for activation of AKT/mTOR signaling to rescue the cells from inhibitory effect of sotorasib (FIG. 3C).

To confirm the role of ITGB4 in sotorasib resistance, ITGB4 were overexpressed in H23 cells in which ITGB4 was expressed at a basal level and generated a stable cell line with increased expression of ITGB4. Treatment of the stable cell line was done with increasing concentrations of sotorasib and the drug effect on proliferation as described above was determined. The parental cells were 50% and 70% inhibited in presence of 5 and 10 μM concentration of sotorasib, respectively, whereas the ITGB4-overexpressing H23 cells were only 5% inhibited at 10 μM concentration of sotorasib Indeed, ITGB4 overexpression could rescue the cells from the inhibitory effect of sotorasib even at the highest concentration (10 μM) of the drug (FIG. 4B).

Next, to discern the effects on the downstream AKT/mTOR signaling pathway, these cells were treated with sub-IC50, IC50, and 2X-IC50 concentrations of sotorasib and collected the lysate after 72 hours of drug treatment and determined the levels of the individual proteins by immunoblotting (FIG. 3D). The data confirmed the instability ITGB4 in presence of sotorasib. Further, there was a decrease in expression of PXN, phospho-ERK, cyclin D1, CDK2, and β-actin in the parental H23 cells. However, in the ITGB4-overexpressing cells, the decrease in the levels of these markers was rescued and the activation of phospho-AKT and phospho-p70 S6 kinase was persistent. Thus, ITGB4 overexpression was unable to rescue the activation of ERK. These results provide evidence implicating ITGB4 in the activation of the AKT/mTOR pathway (FIG. 3D).

To validate the correlation of ITGB4 with sotorasib resistance, isogenic resistant cells were generated by continuously treating them with their respective IC50 concentrations until they developed resistance. Isogenic resistant cell lines were used and comparison of the expression of ITGB4, FAK, and PXN were performed between the untreated or treated cells by immunoblotting. The data confirmed that the cells which develop resistance to sotorasib, namely H23 and SW1573 have increased expression of ITGB4. Interestingly, H358 cells with an IC50 63 nM, could not be made resistant to sotorasib. And this cell line consistently showed a decrease of expression of ITGB4 (FIG. 4C).

The proteasome inhibitor carfilzomib acts synergistically with sotorasib: In previous study, it was shown that the FDA-approved proteasome inhibitor carfilzomib suppresses the expression of ITGB4 and sensitize the KRAS G12A mutant cell line to platinum therapy. This observation prompted us to identify the inhibitory effect of carfilzomib and sotorasib in the SW1573 cell line. We used 8 different concentrations of sotorasib and carfilzomib in the form of a matrix to determine the synergistic effect of the drug combination. It was observed that, at the lowest concentration of 10 μM, carfilzomib alone had a 20% inhibitory effect and sotorasib had about 10% inhibition at a concentration of 16 μM whereas, cells treated with a combination of 16 μM of sotorasib and 10 nM of carfilzomib showed approximately 60% inhibition in cell growth demonstrating that the 2 drugs act synergistically (FIG. 4E). The synergy finder codes were used to determine the synergy between drugs for 48 possible combinations. The codes were run using the R-platform and synergy was calculated and represented as the synergy landscape. The same program was used to determine the synergy score and the highest score was calculated to be 30 for the combination of 8 μM sotorasib and 10 nM of carfilzomib (FIG. 4D). The experiment was repeated by keeping the carfilzomib concentration (10 nM) constant, while increasing the concentration of sotorasib and the drug inhibitory effect was analyzed after 72 hours of treatment. At a low concentration of sotorasib, carfilzomib had an antagonistic effect. At near IC50 concentration, carfilzomib had an additive effect, and at concentrations greater than IC50, carfilzomib had a synergistic effect (FIG. 4E).

The experiments using a constant concentration of carfilzomib and increasing sotorasib were repeated and the cells were harvested after 72 hours of treatment to determine the changes in signaling. As indicated in the cell proliferation assay, the presence of carfilzomib induced strong apoptosis and DNA damage at concentrations of 12 μM and 16 μM, and there is marked reduction in expression of ITGB4 and activated AKT. Thus, these data demonstrate that carfilzomib can be used to inhibit ITGB4 and sensitize sotorasib refractory tumors (FIG. 4F).

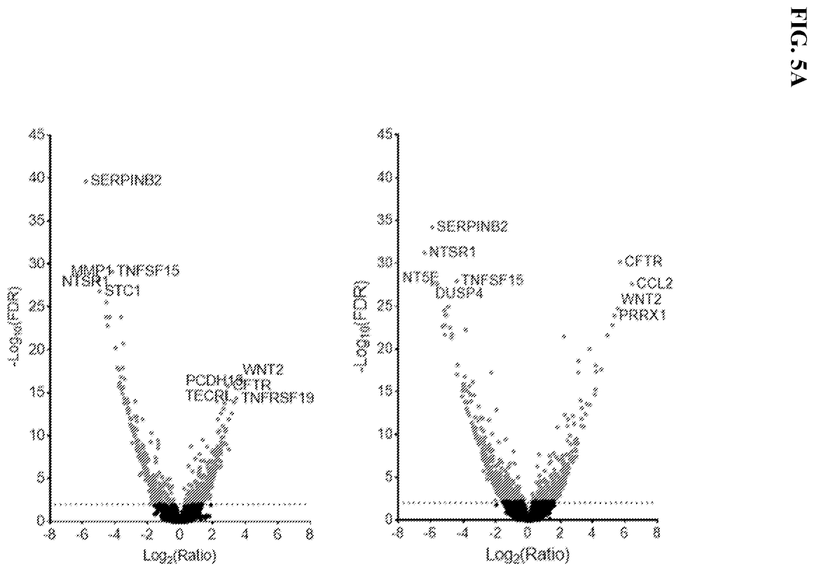

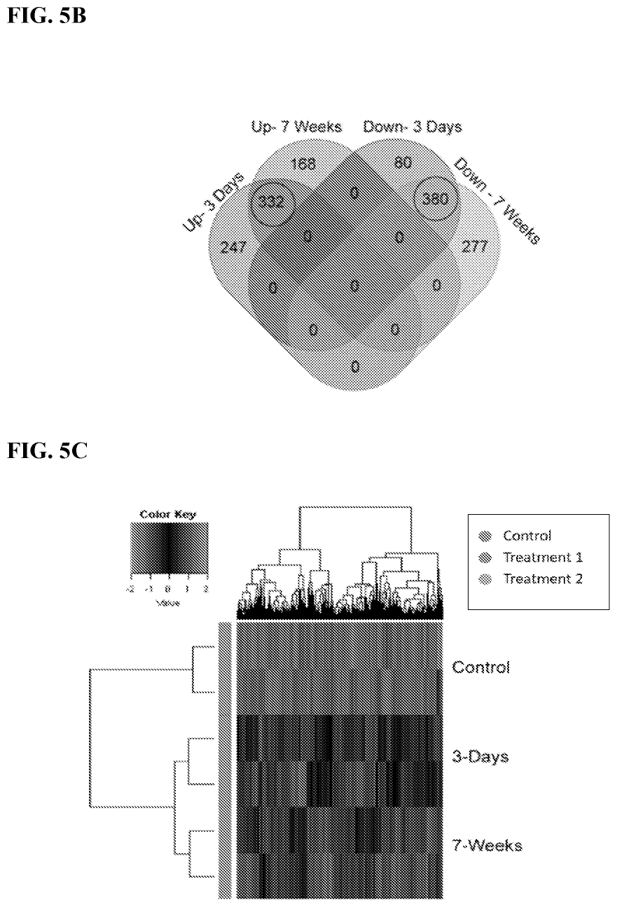

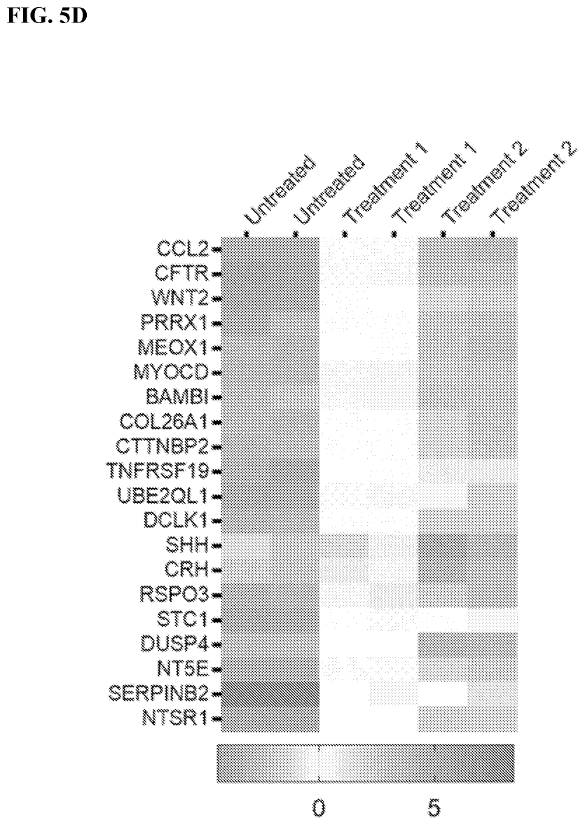

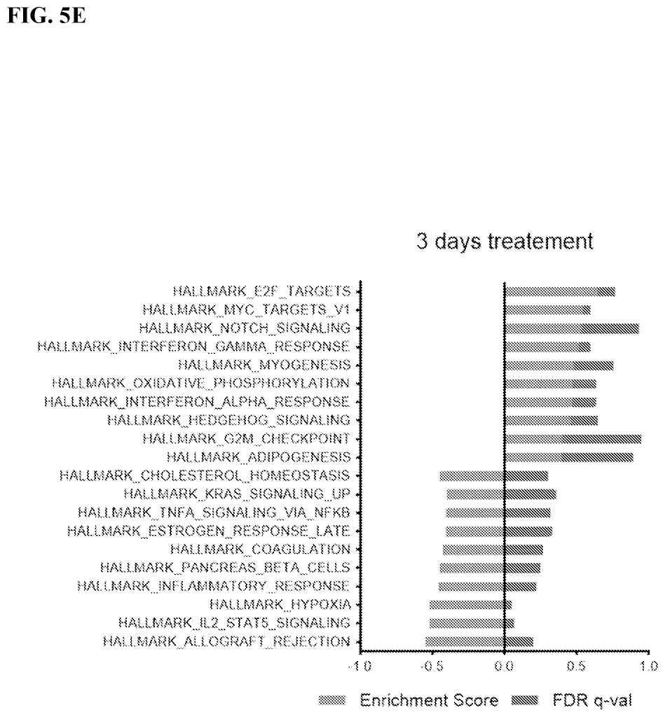

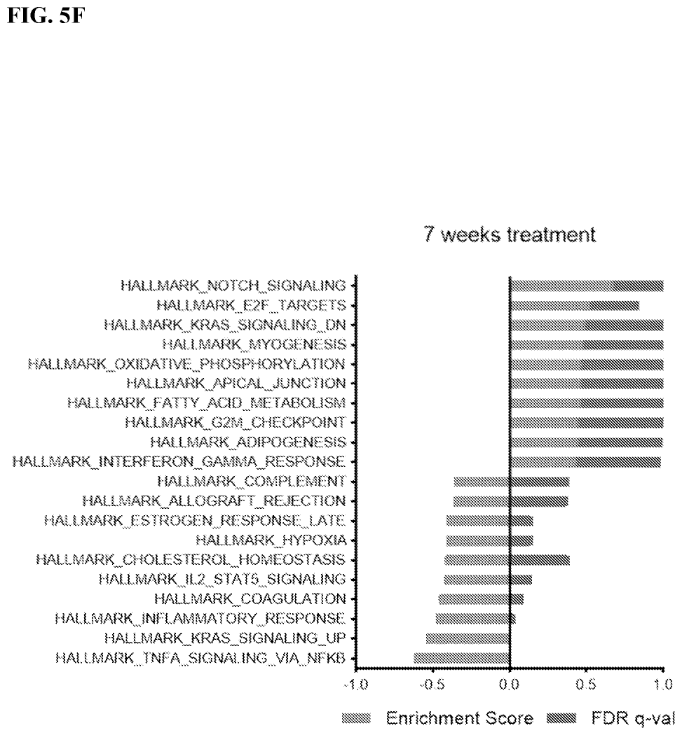

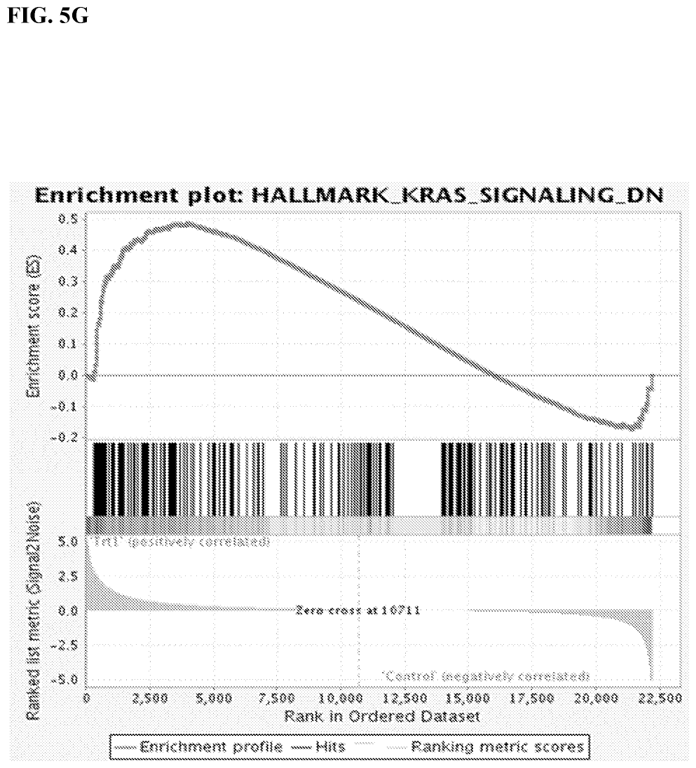

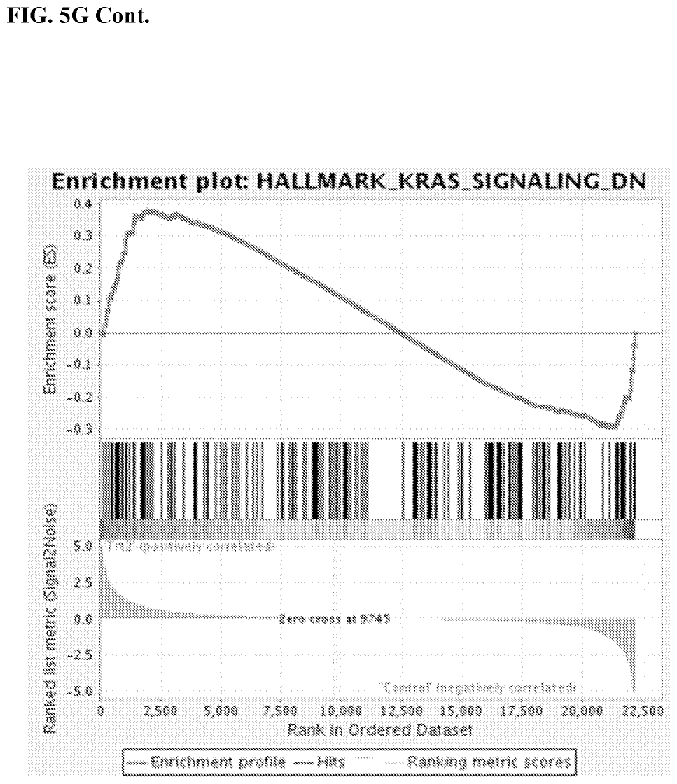

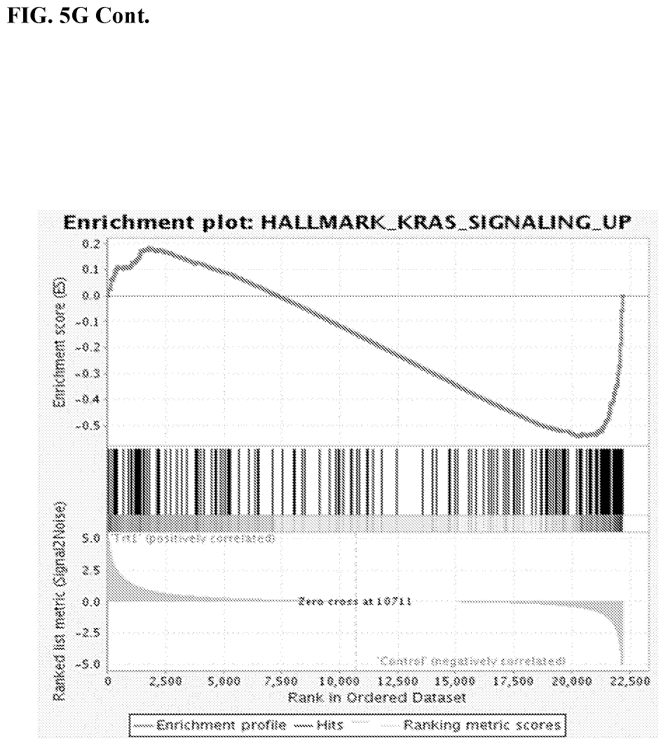

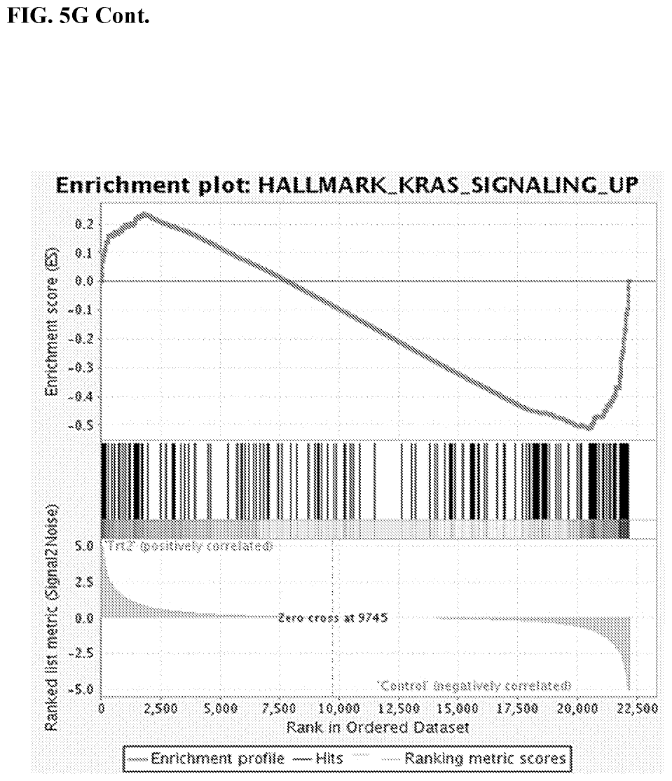

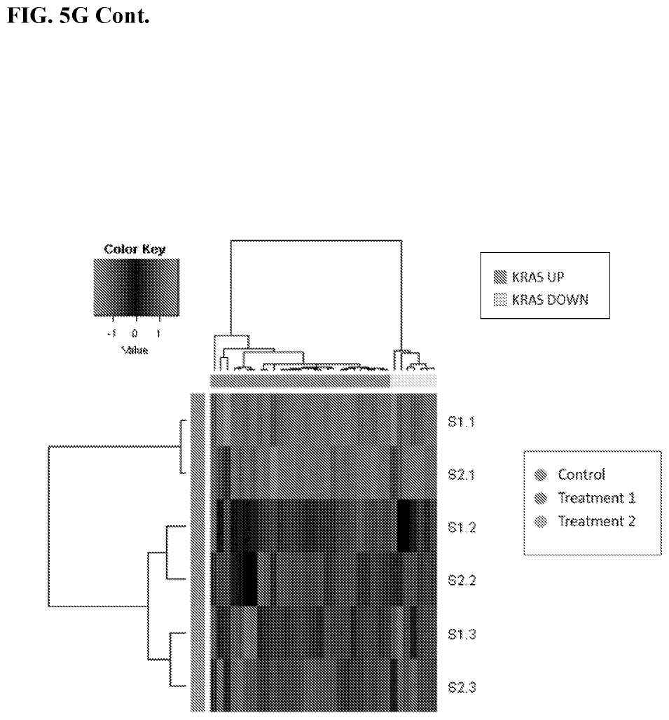

Upregulation of WNT2, CFTR and CCl2 genes in isogenic sotorasib resistant cell line: To better understand the mechanism of acquired resistance to G12C inhibitors, selection of the H23 cells were done to develop an isogenic sotorasib resistant cell line. H23 cells were treated for a period of 7 weeks with increasing concentration of sotorasib. Treatment was initiated using a dose of 1 μM sotorasib and eventually raised to 7.5 μM. RNA was extracted from the drug resistant cells as well as from the cells treated with IC50 dose of the drug for 3 and 7 days and RNAseq analysis was performed. Untreated cells were used as the control and the data were visualized using a volcano plot (FIG. 5A). A set of 332 genes were upregulated in both the treatments and a set of 380 genes were downregulated in both the treatments (FIG. 5B). A clustering analysis of these 712 genes was performed (FIG. 5C) and the top 10 genes which were consistently upregulated in both treatment 1 and treatment 2 were, CCL2, CFTR, WNT2, PRRX1, MEOX1, MYOCD, BAMBI, COL26A1, CTTNBP2, and TNFRSF19. Similarly, the list of genes which were consistently downregulated comprised UBE2QL1, DCLK1, SHH, CRH, RSPO3, STC1, DUSP4, NT5E, SERPINB2, and NTSR1 (FIG. 5D). A gene set enrichment analysis was performed for both treatments and the data were visualized from high enrichment score to low enrichment score (FIGS. 5E-5F). The genes representing E2F targets and G2M checkpoint were consistently upregulated for both the treatments. As expected, the enrichment score for the hallmark-KRAS signaling-up was negative and the hallmark-KRAS signaling-down was positive for both the treatments (FIG. 5G). The top 3 genes were selected, namely CCL2, WNT2, and CFTR for further validation using qPCR TaqMan Probe assay. The results confirmed significant upregulation of these gene expression upon short-term exposure to sotorasib and further upregulation upon continuous exposure to sotorasib for 7 weeks (FIG. 5H). Using Syber Green qPCR assay, it also validated the expression of the other genes listed in FIG. 5D, in reference to gene expression of the untreated cells, refer to FIG. 5I. Next, the 3 cell lines were treated with half IC50 dose and IC50 dose of sotorasib for 3 days and probed the harvested cell lysates by immunoblotting. The blots showed upregulation of WNT2 protein upon exposure to sotorasib in H23 and H358 cell lines, whereas in the SW1573 cells high expression of WNT2 was evident even in untreated cells (FIG. 5J). Mining of the lung cancer TCGA database revealed that Wnt2 expression is lower in tumor tissue compared to normal tissue. However, some studies reported the aberrant activation of Wnt2 autocrine signaling activation in about 50% of NSCLC primary tumors and cell lines. (Ref 33). Therefore, the functional significance of the Wnt signaling pathway in sotorasib resistance or sensitivity warrants further investigation.

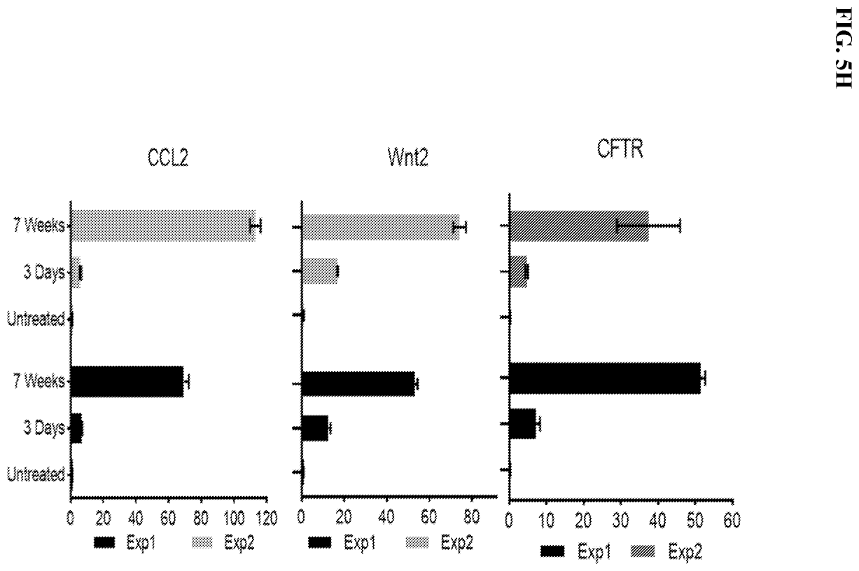

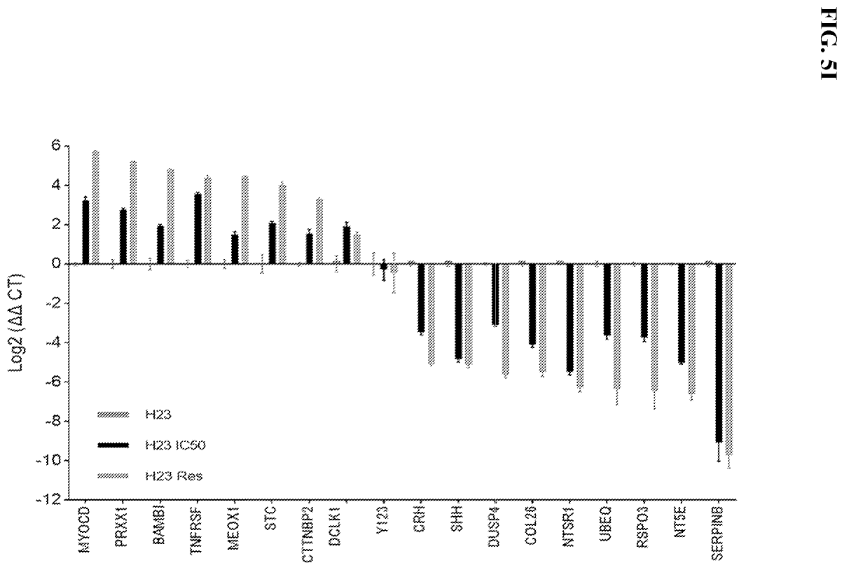

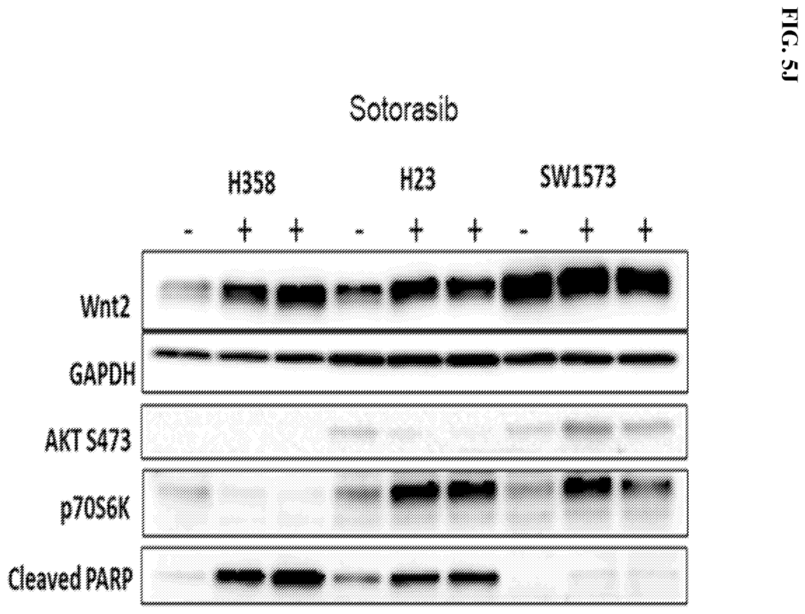

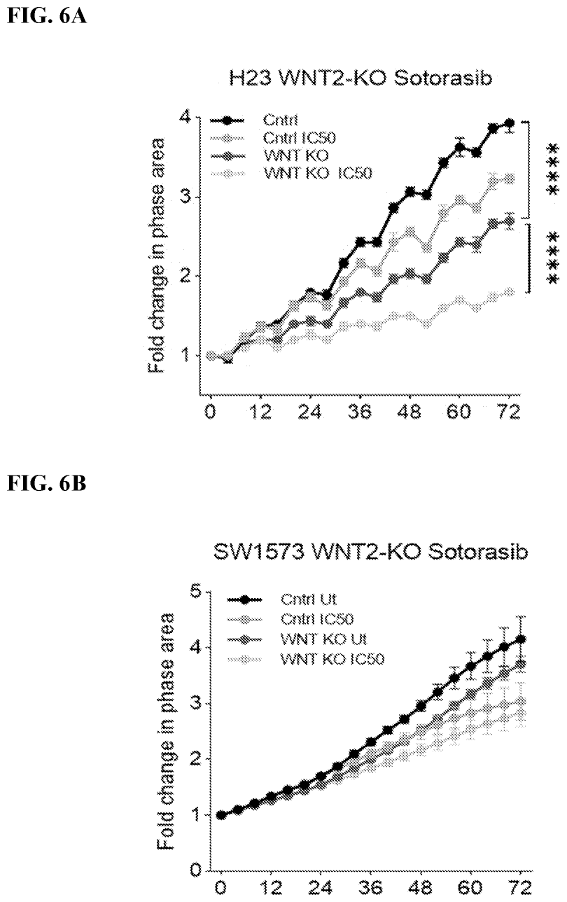

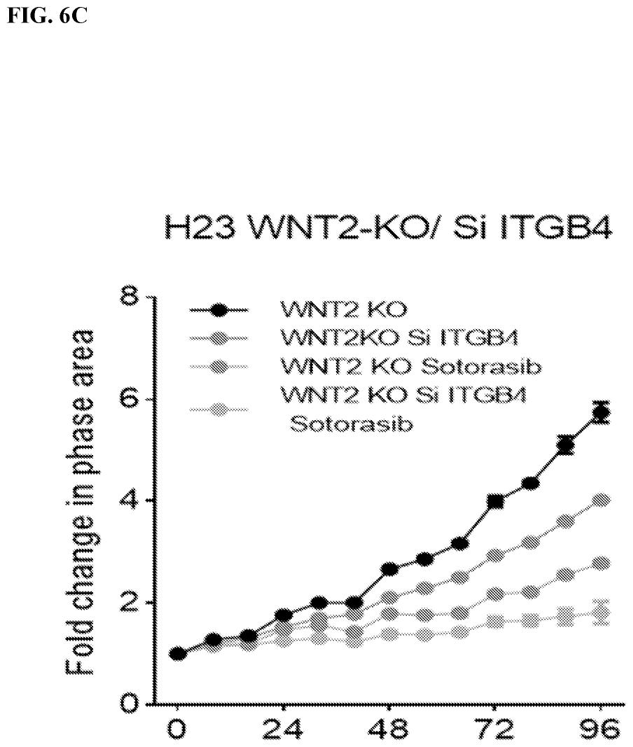

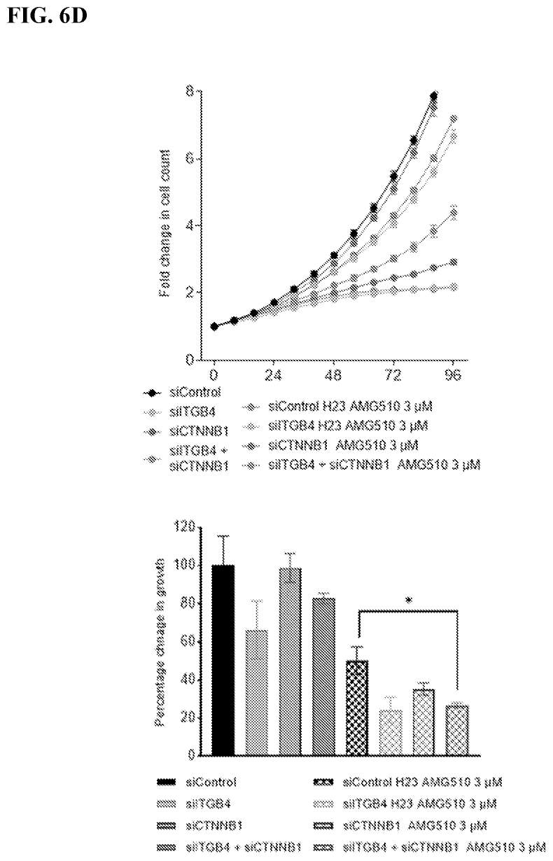

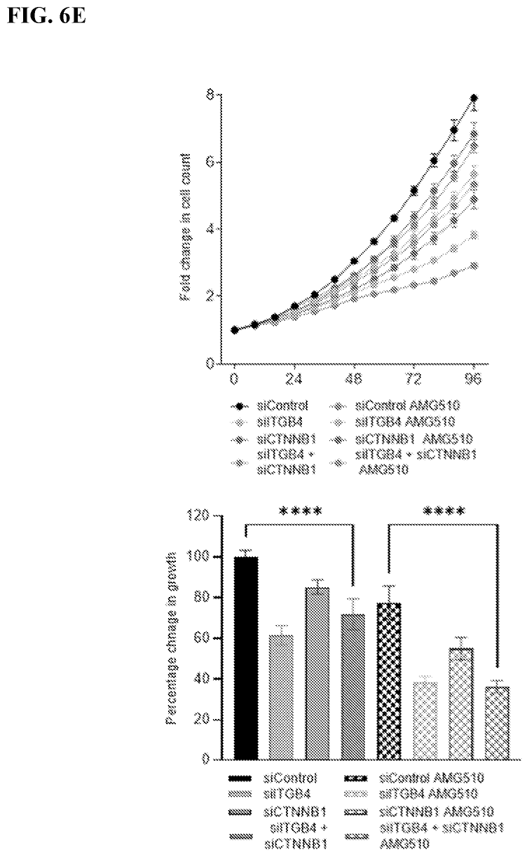

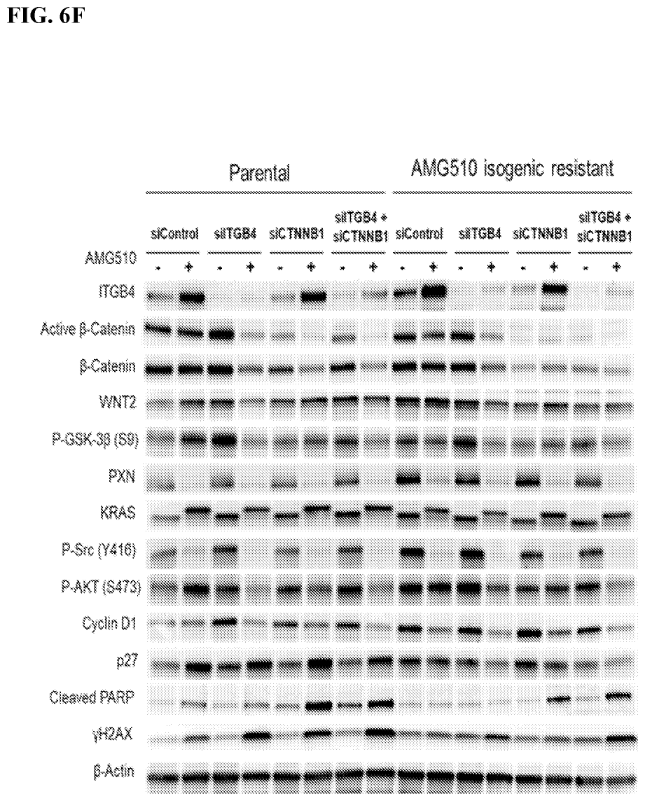

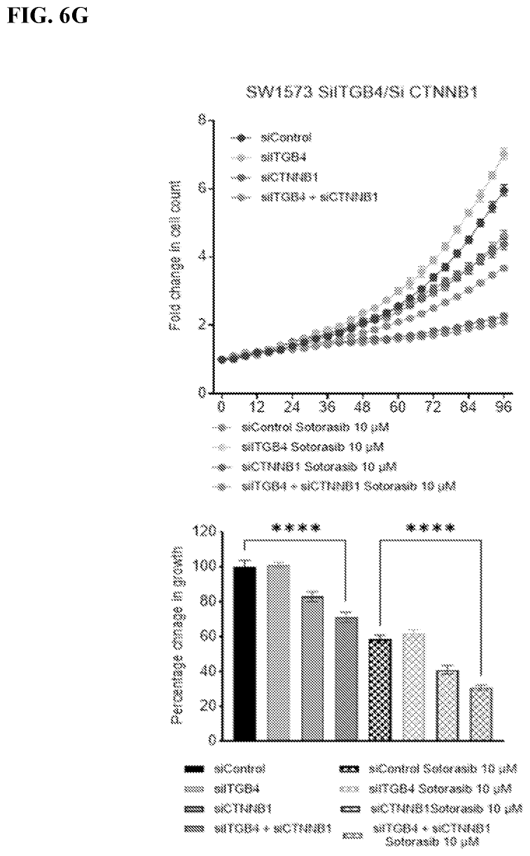

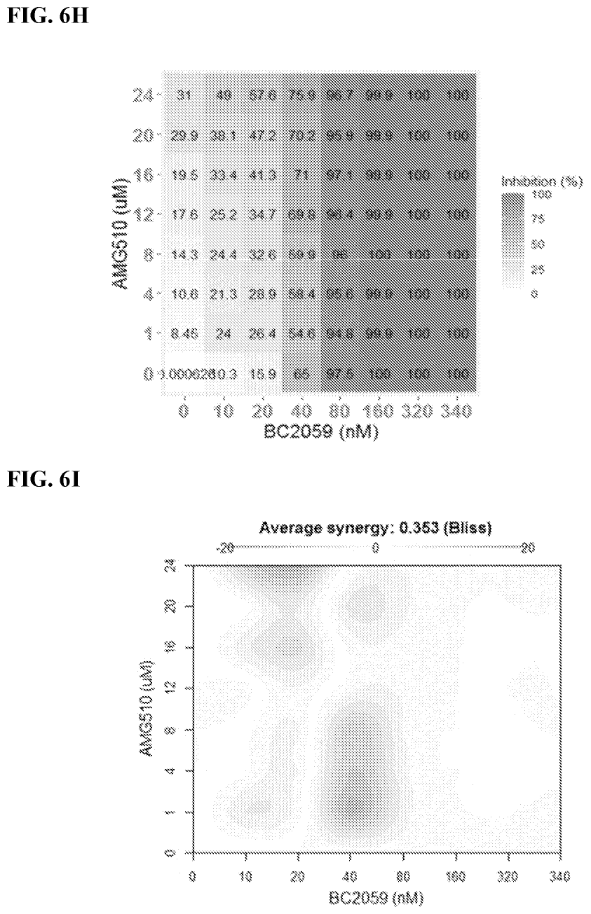

WNT2 knockout sensitizes cells to Sotorasib: Using a CRISPR/Cas9 plasmid against the WNT2 loci, WNT2 knockout SW1573 and H23 cell lines were generated and determined their sensitivity to sotorasib by performing the live cell proliferation assay. According to observation, WNT2 knockout alone is sufficient to inhibit cell proliferation in H23 cells and addition of sotorasib further suppressed cell growth (FIG. 6A). Next, the H23 parental and WNT2 knockout cell lines were used and treated them with increasing concentrations of sotorasib to determine signaling changes. It was observed that ITGB4 activation was suppressed in the WNT2 knockout cells in addition to increased γH2AX and a decrease in active β-catenin and cyclin D1 (FIG. 5A). However, in SW1573 cells any significant change was not observed in cell growth compared to the control both in the absence and presence of sotorasib (FIG. 6B). Interestingly, according to the cell line database (Cellosaurus) SW1573 cells have a β-catenin mutation which can induce it's de novo activation and stability. Next, it was further asked whether knocking down ITGB4 in the WNT2 knockout H23 cells would sensitize them. H23 WNT2 knockout cells were transfected with an ITGB4 siRNA followed by treatment with the IC50 concentration of sotorasib and discerned the effect on cell proliferation in real time. Indeed, ITGB4 knockdown further inhibited cell growth upon addition of sotorasib (FIG. 6C). Thus, the data indicate that presence of β-catenin activating mutation neutralizes the effect of WNT2 knockout in the SW1573 cell line.

Knocking down β-catenin and ITGB4 sensitizes sotorasib resistant cells: H23 parental cell line and H23 sotorasib resistant (20 μM) isogenic line with 10 nM β-catenin siRNA, 10 nM ITGB4 siRNA, or 10 nM β-catenin and 10 nM ITGB4 siRNA were transfected and followed their proliferation with and without sotorasib for 96 hours was studied. The cell proliferation kinetics confirmed that sotorasib treatment inhibits cell proliferation significantly upon knocking down ITGB4 and β-catenin together (FIGS. 6D-6E). The endpoint analysis consistently showed that the double knockdown together with sotorasib sensitizes the isogenic resistant line by 70% (FIGS. 6D-6E). Immunoblotting data using H23 lysates also confirmed the knockdown of total β-catenin and ITGB4 as well as the decrease in expression of active β-catenin. Furthermore, it was observed that treating ITGB4 knockdown cells with sotorasib suppressed the phosphorylation of SRC and AKT kinases, decreased the expression of total and active CTNNB1 and CCND1 while increasing the expression of p27, cleaved PARP and γH2AX. The double knockdown further added to the phenotype as inferred from the stronger activation of p27, cleaved PARP and γH2AX. However, the same signaling analysis done with lysates obtained from sotorasib isogenic resistant cells indicated a weaker phenotype for ITGB4 knockdown in presence of sotorasib but consistently stronger phenotype for the cells with double knockdown (FIG. 6F).

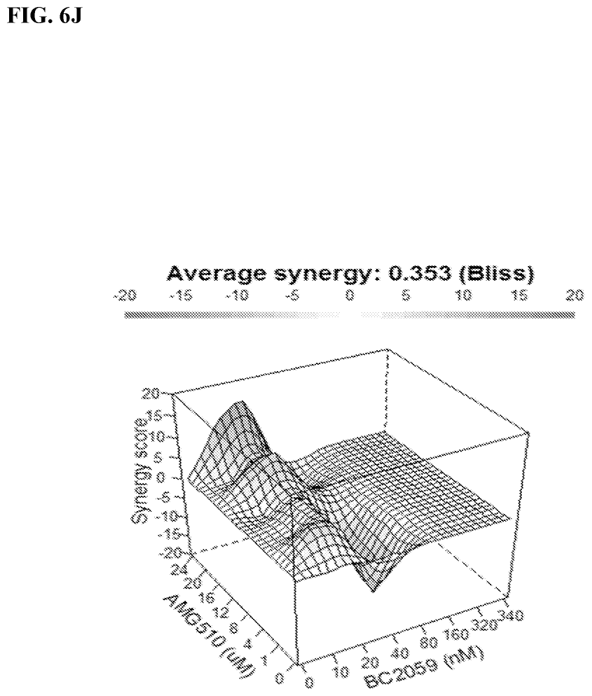

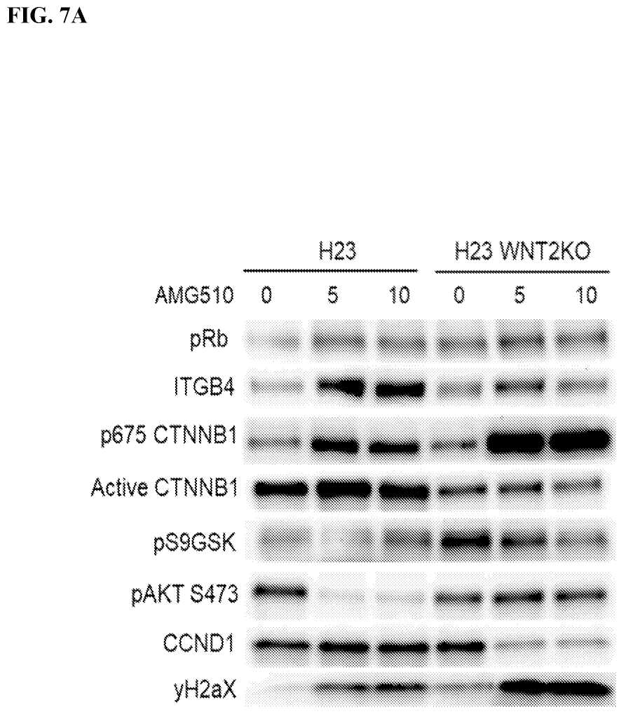

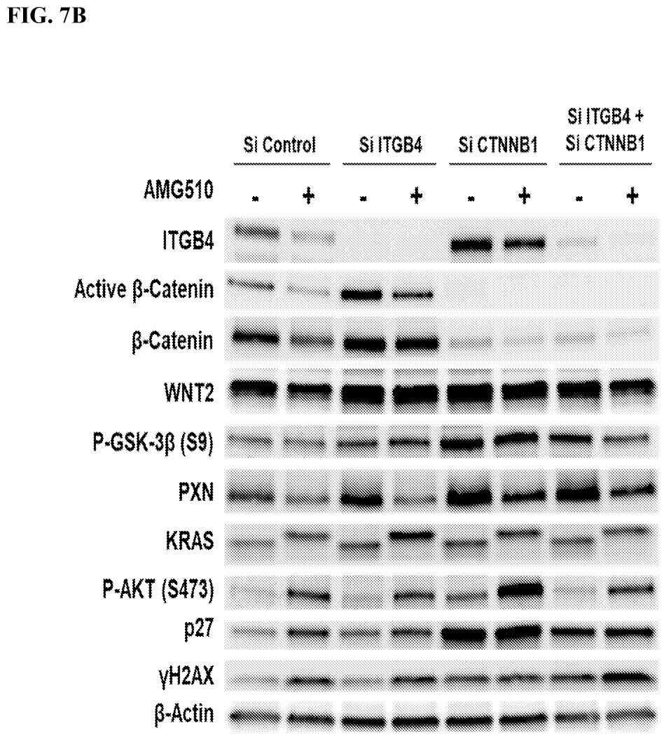

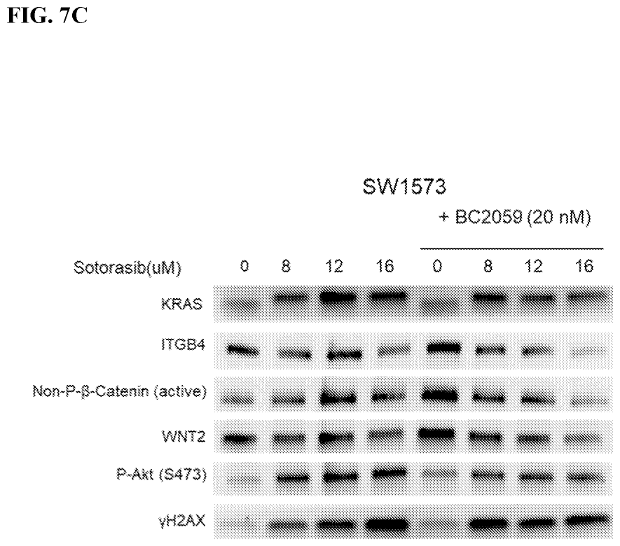

Further, it validated the role of β-catenin and ITGB4 in sensitizing SW1573 cells against sotorasib. SW1573 cells were transfected with 10 nM β-catenin siRNA, 10 nM ITGB4 siRNA, or 10 nM β-catenin and 10 nM ITGB4 siRNA and followed their proliferation with and without sotorasib for 96 hours. The cell proliferation kinetics data revealed that knocking down ITGB4 did not have much effect on SW1573 proliferation, whereas the β-catenin knockdown alone or together with ITGB4 inhibits cell growth by 20%. However, addition of sotorasib at the IC50 concentration induced a cytostatic effect both in β-catenin single or double knocked down cells (FIG. 6G). The percent change of cell growth calculated at the last time point showed a about 40% inhibition for sotorasib only treated cells compared to about 70% inhibition in the double knockdown plus sotorasib treated cells. Thus, targeting ITGB4 and β-catenin together may serve as a potential therapeutic strategy to sensitize sotorasib resistant tumors (FIG. 6G). Next, the cells were harvested after 72 hours of sotorasib treatment and confirmed the knockdown of both β-catenin and ITGB4 by immunoblotting (FIG. 7B). As per observation, cells with double knockdown had weak activation of AKT but strong induction of p27 and γH2AX, indicating poor survival (FIG. 7B).

To validate the β-catenin knockdown experiment pharmacologically, a β-catenin-specific inhibitor, tegatrabetan was used. The inhibition matrix with the combination of sotorasib and tegatrabetan showed that 10 nM of tegatrabetan inhibits 10% of SW1573 cell growth, whereas a combination of 10 nM of tegatrabetan with 1 μM, 16 μM or 24 μM of sotorasib inhibits cell growth by 24%, 33% or 49%, respectively (FIG. 6H). A combination of 10 nM tegatrabetan and 1 μM, 16 μM or 24 μM of sotorasib had a synergistic effect as indicated by the synergy landscape with a synergy score of 15 (FIG. 4I and FIG. 4J). Thus, combining tegatrabetan and sotorasib is a good therapeutic strategy for suppressing the growth of sotorasib resistant tumors. Further, the changes in the signaling in response to the tegatrabetan and sotorasib combination treatment were analyzed by immunoblotting. The data showed decrease in the activation of β-catenin, ITGB4 and AKT, but an increase in γH2AX level supporting the synergism observed with the drug combination (FIG. 7C).

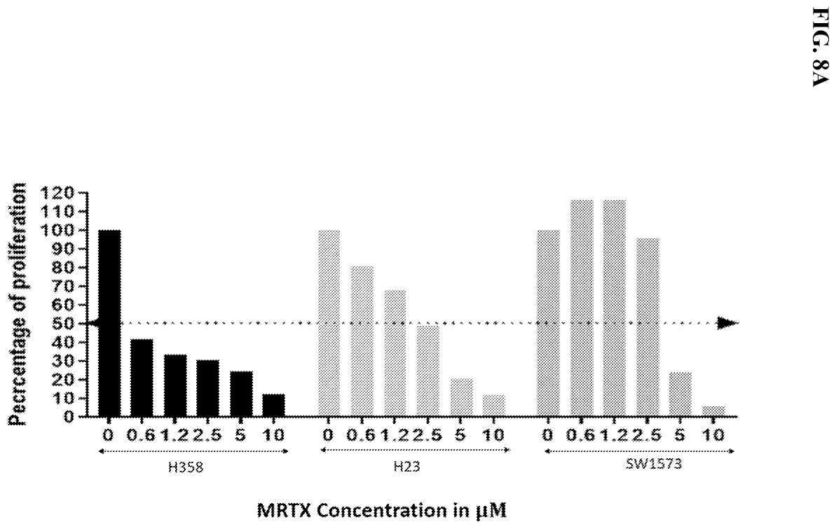

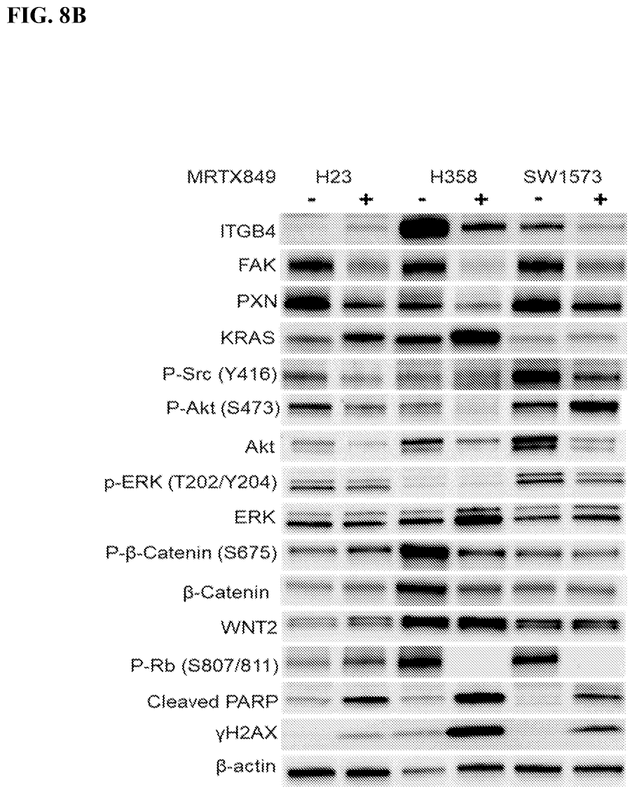

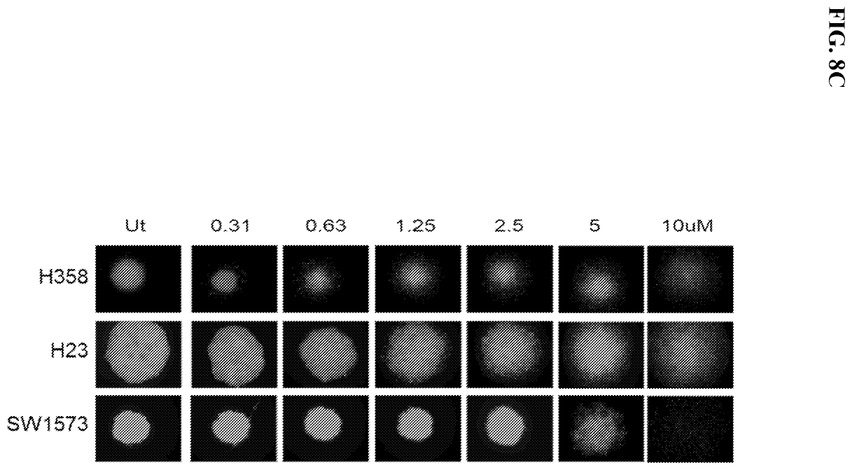

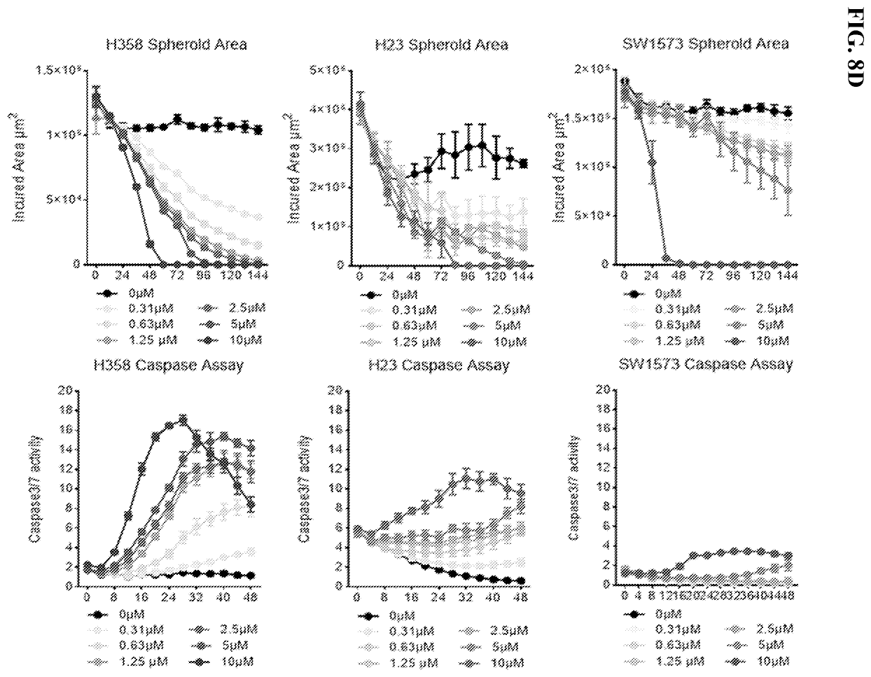

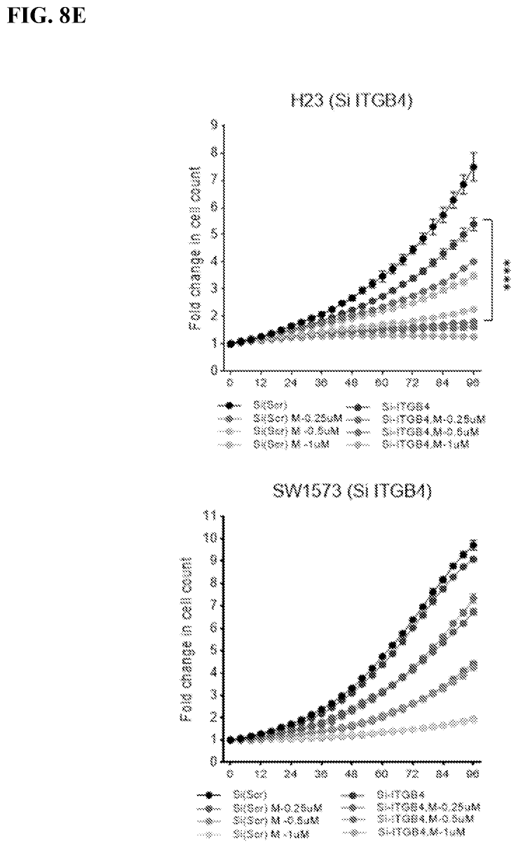

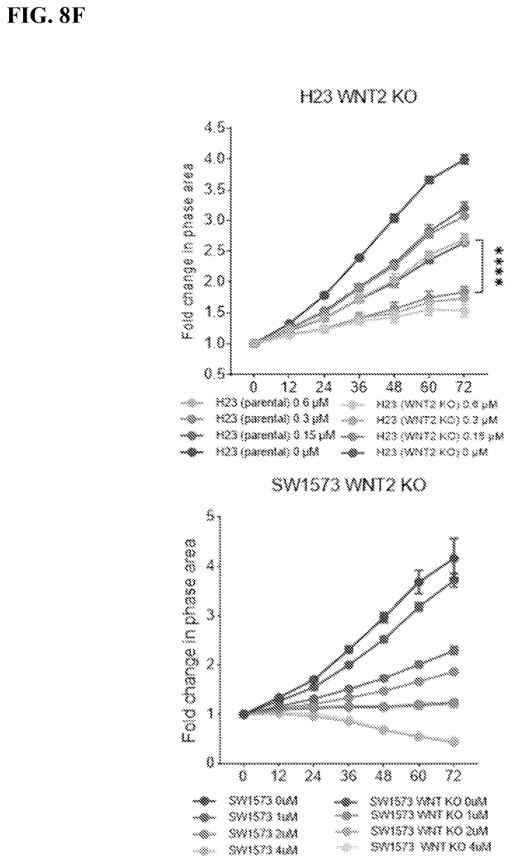

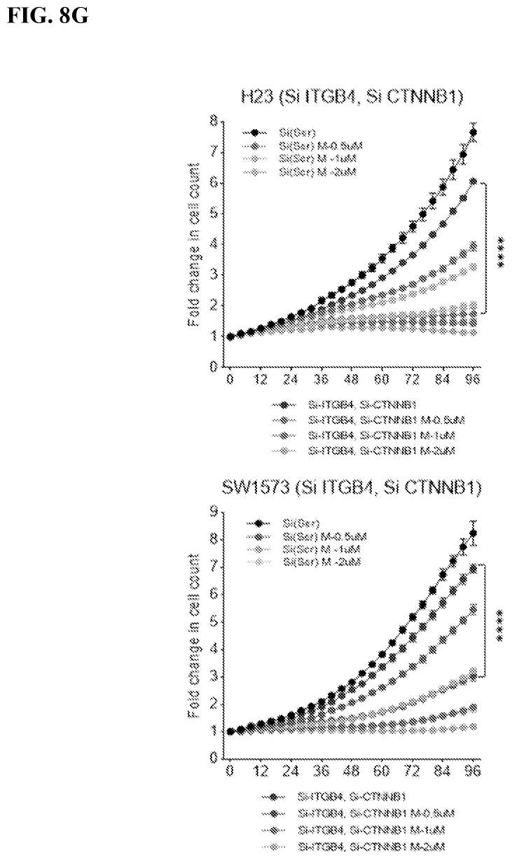

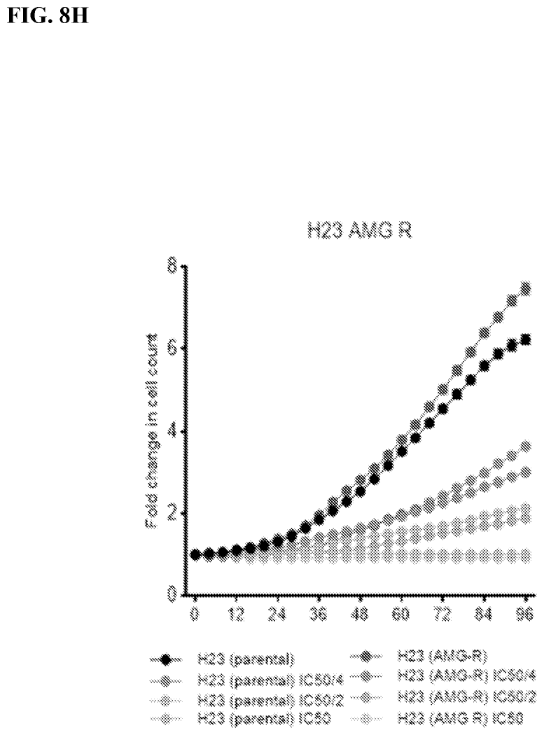

LUAD cells resistant to sotorasib are sensitive to Adagrasib: Adagrasib also covalently binds to the cysteine residue in the KRAS G12C mutant molecule. Therefore, the effect of adagrasib on the sotorasib resistant cell lines were understood. The cell proliferation assay was performed with increasing concentrations of adagrasib using the 3 cell lines mentioned in this study. The data indicated that 5 μM of adagrasib suppresses the growth of SW1573 cells by 80% and almost 95% inhibition was observed at concentration of 10 μM (FIG. 8A). The IC50 concentration for the 3 cell lines, namely H358, H23 and SW1573 were 0.51 μM, 2.15 μM and 4.13 μM, respectively, which are much lower than the IC50 concentrations of sotorasib. Next, evaluation of the signaling changes in response to adagrasib treatment were performed by immunoblotting. The data revealed that adagrasib suppressed the expression of focal adhesion complex-associated proteins ITGB4, PXN and FAK, and also suppressed phosphorylation of SRC kinase, which was not observed upon sotorasib treatment. Further, it was observed a decrease in total levels of AKT, β-catenin, active β-catenin, and phospho-Rb with a simultaneous increase in cleaved PARP and γH2AX. Together these data underscore the differences in the signaling mechanisms by the 2 mutant KRAS inhibitors while highlighting the fact that adagrasib treatment showed a more robust phenotype compared to sotorasib (FIG. 8B).

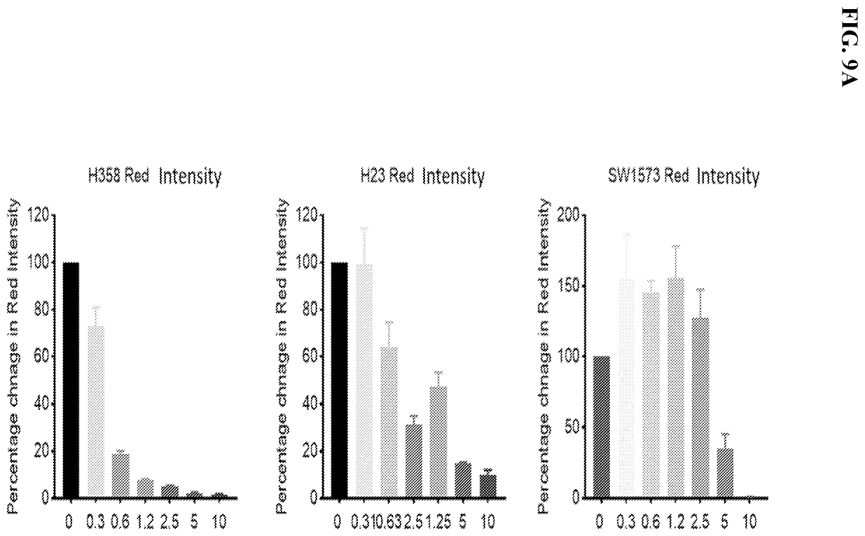

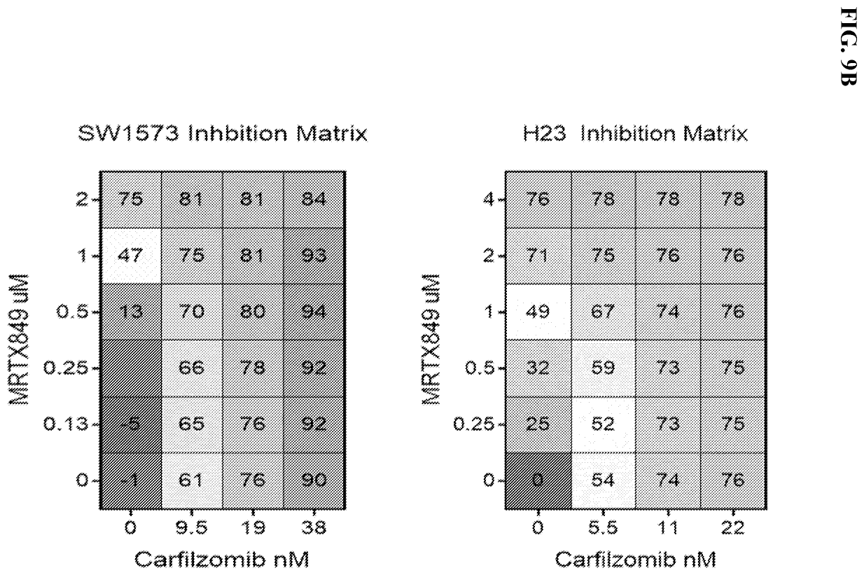

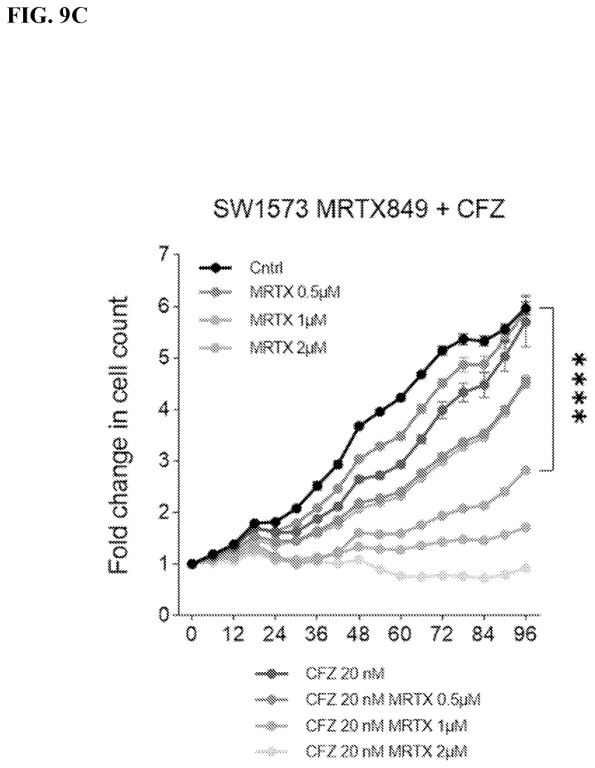

Having established that sotorasib resistant cells are sensitive to adagrasib in 2D cultures, the efficacy of adagrasib in 3D spheroid cultures by determining the changes in spheroid growth and caspase activity was evaluated (FIG. 8C). The spheroid growth kinetics revealed that at 10 μM concentration of adagrasib, spheroid growth is completely abrogated within 48 hours for H358 and SW1573, and by 96 hours for the H23 spheroids which was not the case when spheroids derived from the same cells were treated with sotorasib. Reducing the concentration to 5 μM also abrogated spheroid growth for the H358 and H23 spheroids and reduced the integrity of the SW1573 spheroids (FIG. 8D). In addition, analysis was done for the red mean intensity at the last time points and found that the H358 spheroids were 80% inviable at a minimum concentration of 0.6 μM of adagrasib, whereas the H23 spheroids were only 40% inviable and SW1573 had a 50% better viability compared to untreated spheroids. But consistent with the spheroid area, it was observed that the 10 μM concentration completely suppressed their growth (FIG. 9A). Similarly, it was found that, consistent with the spheroid area and the mean red intensity, caspase-3/7 activity was upregulated within 24 hours of 10 μM adagrasib treatment in H358 and H23 spheroids. For the lower concertation of adagrasib, the caspase activity was upregulated by 48 hours. However, consistent with the previous finding, no significant activation of caspase 3/7 in the SW1573 spheroids alluding to the possibility of multiple apoptotic pathways in these cells were observed (FIG. 8E).

Knocking down ITGB4 and WNT2/β-catenin signaling sensitizes cells to Adagrasib: To determine whether ITGB4 plays any role in manifesting response to adagrasib, ITGB4 using a siRNA was knocked down and cell proliferation was monitored in real time using the live cell imaging system. In untreated H23 cells in which ITGB4 was knocked down, there was a 5-fold increase in cell count compared to an 8-fold increase in the scramble-treated cells. This 8-fold change decreased to 4-fold and 3.5-fold upon 0.25 μM and 0.5 mM treatment of adagrasib, respectively, but the same drug concentrations in the ITGB4 knocked down cells had a cytostatic effect indicating that ITGB4 knockdown supported adagrasib induced inhibition (upper graph in FIG. 8F). Surprisingly, the ITGB4 knockdown did not add much to the adagrasib induced inhibition in the SW1573 cell line, which lead us to explore the effect of WNT2/CTNNB1 signaling in this cell line (lower graph in FIG. 8F).

Then the WNT2 KO H23 and SW1573 cell lines were used to determine the effect of adagrasib. The data revealed a 4-fold increase in cell growth for the control cells and 3-fold increase for the cells treated with 0.15 μM adagrasib. WNT2-KO cells had a weaker proliferation with only a 2.5-fold increase in growth in the absence of adagrasib and in presence of 0.15 μM adagrasib, a 1.5 fold increase in cell count was observed, thus indicating adagrasib treatment on WNT KO H23 cell line affected cell proliferation (upper graph in FIG. 8G). As seen before, WNT2-KO in the SW1573 cell line treated with adagrasib did not induce any significant reduction in cell growth on addition of adagrasib (lower graph in FIG. 8G).

Finally, the effect of β-catenin was determined and ITGB4 double knockdown on response to adagrasib. β-catenin and ITGB4 were inhibited using 10 nM of each siRNA and 24 hours after transfection, cells were treated with various concentration of adagrasib to determine the cell count changes using the live cell imaging system. In β-catenin/ITGB4 double knockdown but untreated H23 cells had a 6-fold change in cell count compared to the 8-fold change for the scramble siRNA treated cells over a period of 96 hours. This change dropped to 4-fold upon 0.5 μM treatment of adagrasib, but the same drug concentration in the double knockdown cells had a significant cytostatic effect (upper graph in FIG. 8H). Similarly, the control SW1573 cells showed a 8-fold change in proliferation, and the double knockdown showed a change of 7-fold. Treatment with 0.5 μM or 1 μM of adagrasib suppressed the growth to ˜5 and 3-fold, respectively. The same concentration in the double knockout cell line inhibited growth to 3-fold or 1.5-fold respectively. The data demonstrate that targeting ITGB4 and β-catenin together can inhibit the growth of sotorasib resistant SW1573 cells harboring the β-catenin mutation (upper graph in FIG. 8H).

Carfilzomib and adagrasib act synergistically at low concentrations: To determine if carfilzomib and adagrasib can act synergistically, the following study was performed. After 72 hours of drug treatment it was observed that 10 nM of carfilzomib was able to inhibit growth of the SW1573 cells by 60% and addition of 1 μM of adagrasib had an additional effect leading to 75% of inhibition compared to 47% of inhibition by adagrasib alone. A similar effect was also observed for the combination of 1 μM adagrasib and 5.5 nM of carfilzomib in the H23 cell line. But in this case, either increasing (2 μM) or decreasing (0.5 μM) adagrasib concentration had no significant effect (FIG. 9B).

Next, it was sought to determine cell proliferation changes with the combination treatment. CFA at a concentration of 20 nM had a lower proliferation curve at early time points but at the end point, the proliferation curve merged with the curve representing the control, demonstrating that carfilzomib slows cell growth. Similarly, a combination of adagrasib (1 μM) and carfilzomib (20 nM) slowed cell growth and restricted it to a 3-fold increase compared to the 5-fold increase by 1 μM of adagrasib alone. At 2 μM concentration, adagrasib had a cytostatic effect and in combination with carfilzomib further suppressed cell growth indicating the drop was due to cell death (FIG. 9C). Next, we validated the inhibitory effect adagrasib by determining drug inhibitory effect on the sotorasib isogenic resistant lines (resistant to 20 μM sotorasib). The data clearly showed that adagrasib significantly inhibited the growth of these cell lines even at lower concentrations. Thus, the data demonstrate that adagrasib can inhibit the growth of the sotorasib resistant cell lines alone; and in combination with carfilzomib induced a stronger inhibition.

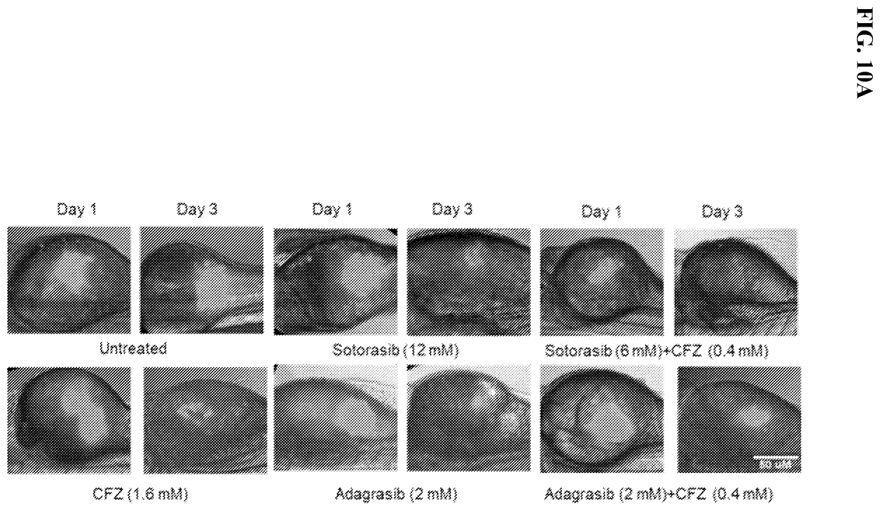

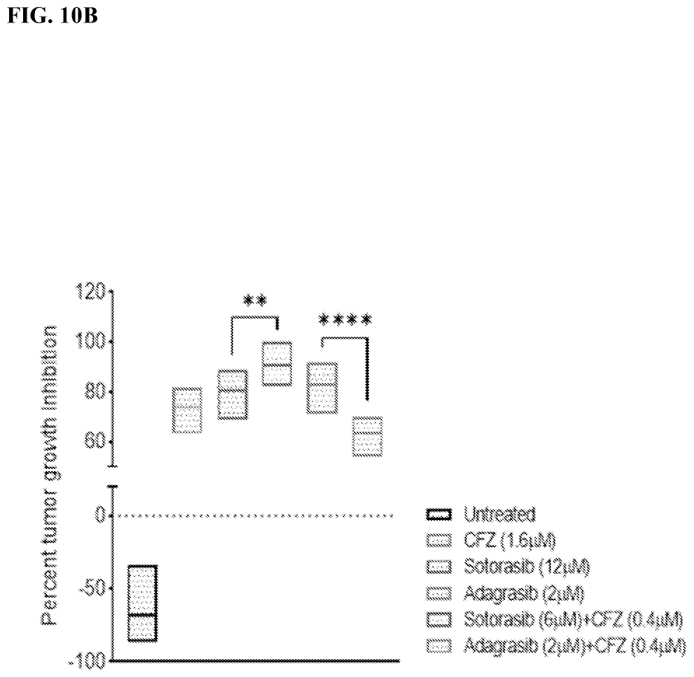

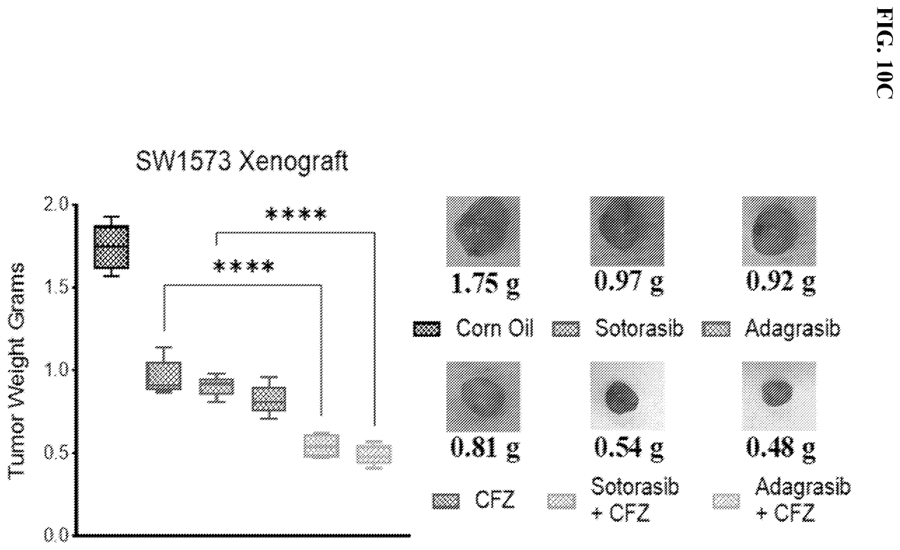

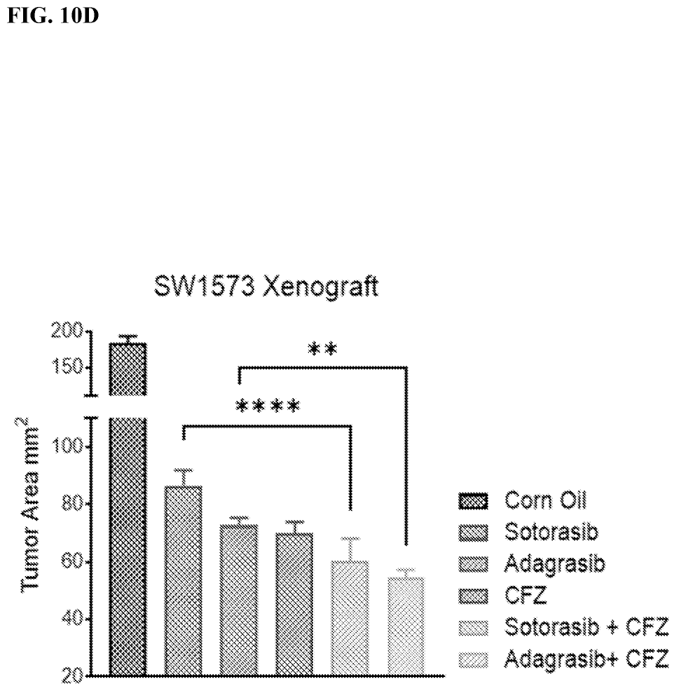

Carfilzomib and adagrasib act synergistically in vivo: Having confirmed the synergistic effect in 2D and 3D cell line models, it was set out to determine the synergistic effects in vivo. For this purpose, H23 cells was firstly used to create zebrafish xenotransplants. Dil green-labelled cells were injected into the perivitelline space of anesthetized 48-hpf (hours post fertilization) larvae as described in the methods. Twenty-four hpi (hours post injection), the larvae were distributed in a 96-well plate. The maximum tolerated dose (LD50) for sotorasib, adagrasib, and carfilzomib was determined by adding the drug to the fish water in which the larvae were grown. The larvae were allowed to grow for 72 hours and the toxic effects of the drugs on regular growth and development were assessed by examining the length and shape of the body. To determine the effects of the drug on the tumor cells, larvae were monitored for tumor size using a Zeiss Observer 7 fluorescent microscope (5× objective) at Day 0 and Day 3 (FIG. 10A). Treating the larvae with sotorasib (12 μM), adagrasib (2 μM) and carfilzomib (1.6 mM) resulted in about 80%, 75% and 82% (median) reduction of tumor growth, respectively. Sotorasib and carfilzomib acted synergistically when used in a combination, as the drug requirement reduced to 2-4 times and only 6 μM sotorasib and 0.4 μM of carfilzomib were required to see the same effect. However, unlike sotorasib, adagrasib alone was more effective in terms of tumor reduction when compared to combination treatment of adagrasib and carfilzomib (FIG. 10B).

For mouse xenograft studies, a 100 μl suspension containing 1×106 SW1573 NSCLC cells was injected subcutaneously (s.c.) on right flank of nude mice above the hind limb, and the antitumor effects of sotorasib, adagrasib, carfilzomib, and their combinations were examined (FIG. 11A). Animals were treated with corn oil, sotorasib, adagrasib or carfilzomib by oral gavage. The average weight gain among the 6 groups was similar, demonstrating that the drugs had no gross toxic effects (FIG. 11B). Compared to the control group, adagrasib, sotorasib and carfilzomib treatment caused a significant reduction in tumor weight measured at the end of study (day 56) (FIG. 10C). Treatment with sotorasib, adagrasib or carfilzomib alone caused about 52%, 58% and 56% reduction in tumor area, respectively. A greater reduction in tumor area was observed in animals that received combinatorial treatment. Thus, we observed about 65% with the sotorasib+carfilzomib combination and about 69% with the adagrasib+carfilzomib combination (FIG. 10D). No macroscopic evidence of metastasis to other organs was evident for any experimental groups. Together, these results demonstrate that sotorasib or adagrasib and carfilzomib combination therapy is a therapeutic strategy for NSCLC, especially in sotorasib resistant lung cancer.

Despite targeting the same cysteine residue, adagrasib and sotorasib exhibit significant differences in their mechanism of action. As per observation, adagrasib alone can suppress the growth of sotorasib resistant cell lines indicating that mechanisms underlying the inhibitory effect of the 2 drugs may be quite different. To rest this hypothesis, H23 cells were treated with increasing concertation (1-5 μM) of sotorasib or adagrasib and monitored the effect on cell proliferation. The control cell count increased from 9,000 to 15,000 over a period of 24 h. In the presence of 1-3 μM of sotorasib, the cell count increased to 12,000. However, at a concentration of 5 μM, sotorasib was cytostatic and caused cell growth arrest. In contrast, cytostatic effect was observed at a minimal concentration (1 μM) of adagrasib. Increasing adagrasib concentration from 2 to 5 μM led to a drop in the cell count from 9,000 to about 4,000. These data underscore the differential behavior of the two inhibitors and demonstrate that adagrasib can induce cell death (FIG. 10E).

Adagrasib induces marked cell cycle arrest. Using commercially available lentiviral particles (Essen Bioscience Inc.), a SW1573 stable cell line was developed to determine the dynamics of the cell cycle in response to the KRAS inhibitors. In the G1 phase, these cells express the red fluorescent protein and in G2, they express the green fluorescent protein, and cells transitioning from G1 to G2, express both the proteins giving rise to yellow florescence. Cells were treated with increasing concentration of sotorasib from 1.25 to 20 μM, or adagrasib from 0.6 to 10 μM. After 72 hours of treatment, the cell cells were sorted by FACS. Increasing concentration of sotorasib pushed the cells to G1 arrest and at 20 μM of sotorasib, the G1 population increased from about 39% to 62%. Simultaneously, there was a drop in the G2 population from 26% to 11%. In contrast, 0.6 μM or 1.25 μM of adagrasib was sufficient to induce G1 arrest comparable to that seen with 20 μM of sotorasib. On further increasing the concentration of adagrasib 5 μM, the G1 population dropped from 64% to 32% (FIG. 10F). These observations demonstrate that adagrasib induced toxicity leads to cell death. Further, the cycle analysis demonstrates that increasing adagrasib concentration caused reduction in G1 population. Thus, adagrasib is inducing cell death in the G1 arrested cells.

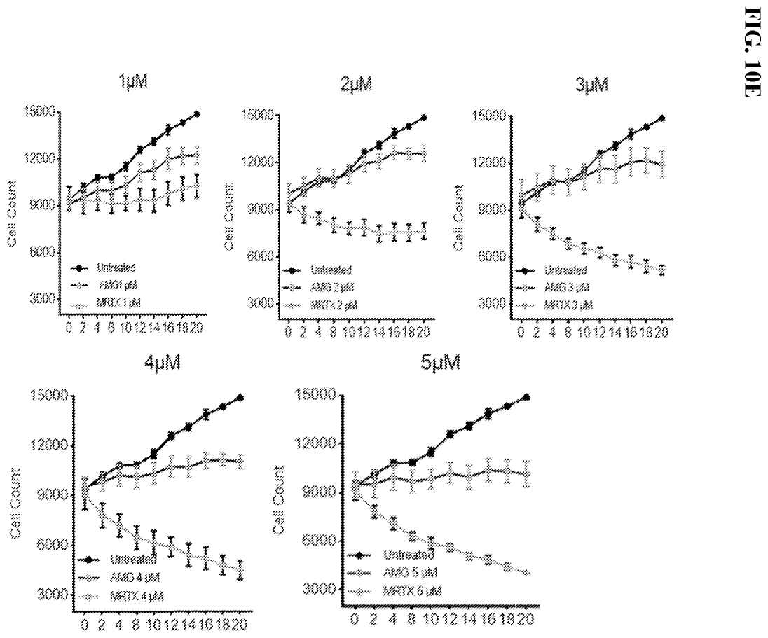

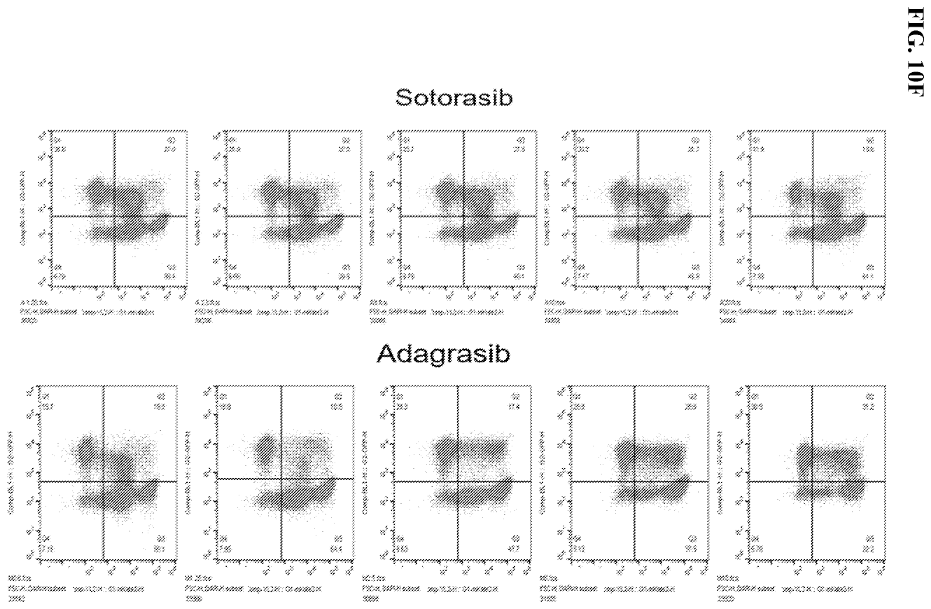

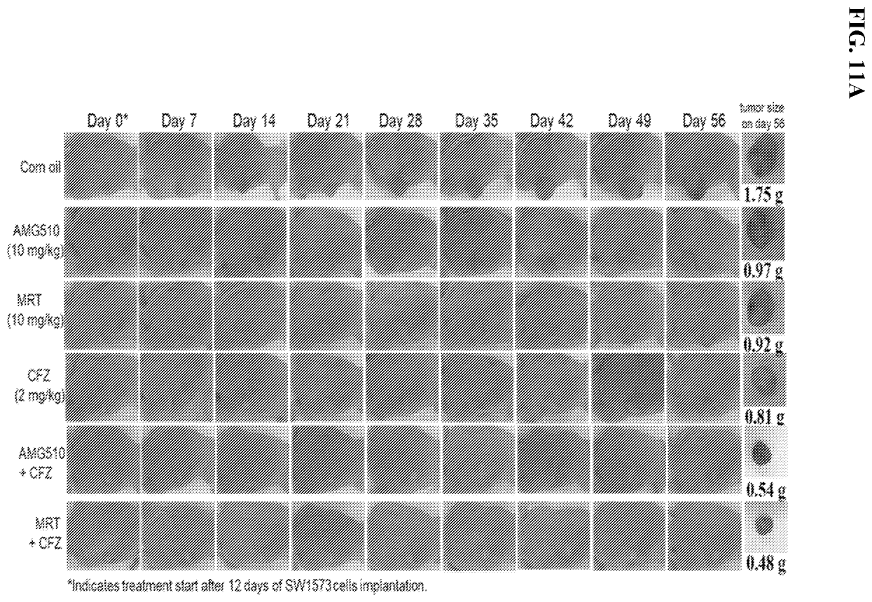

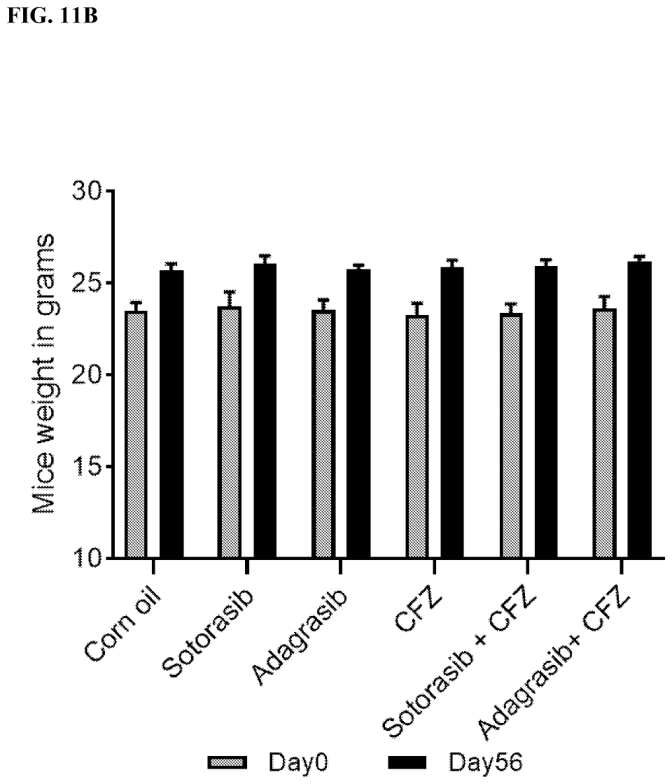

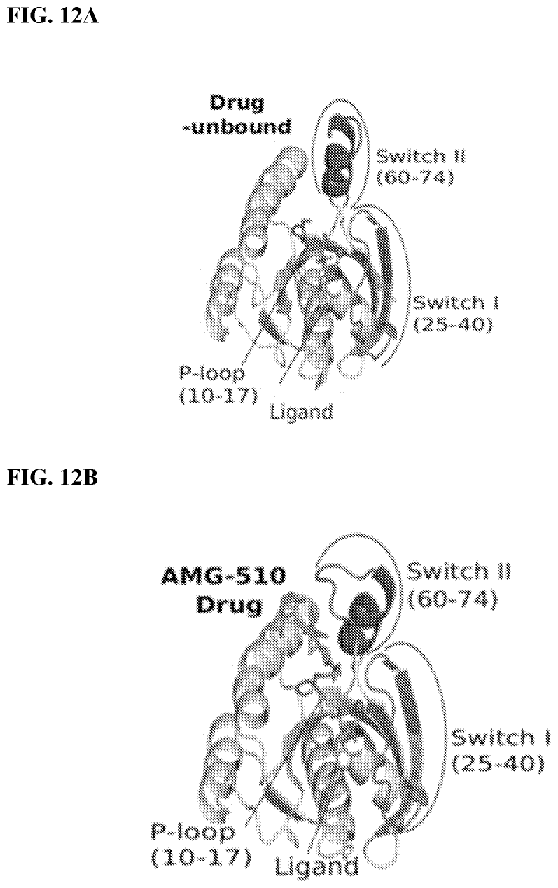

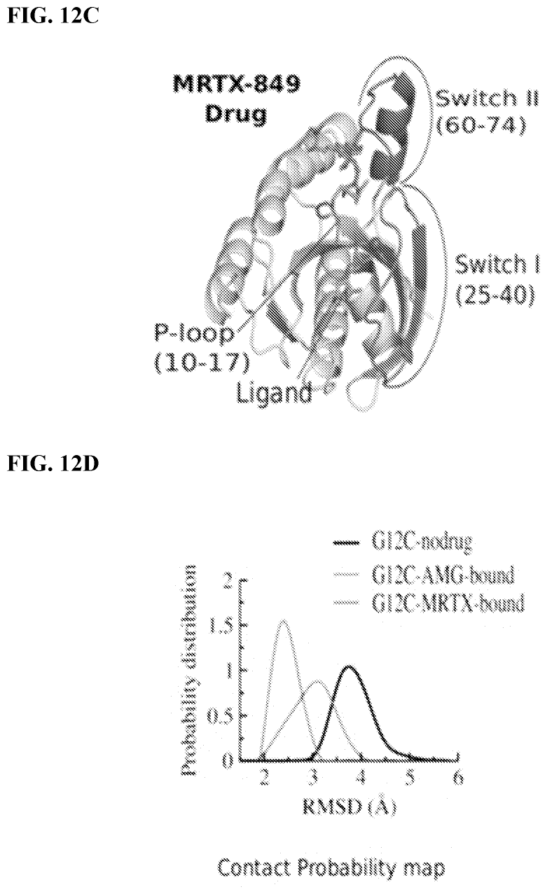

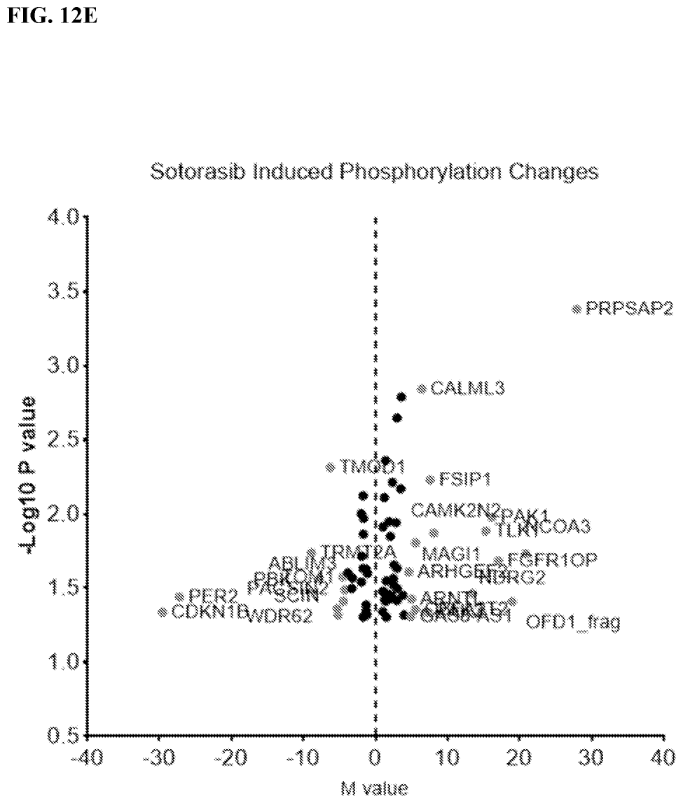

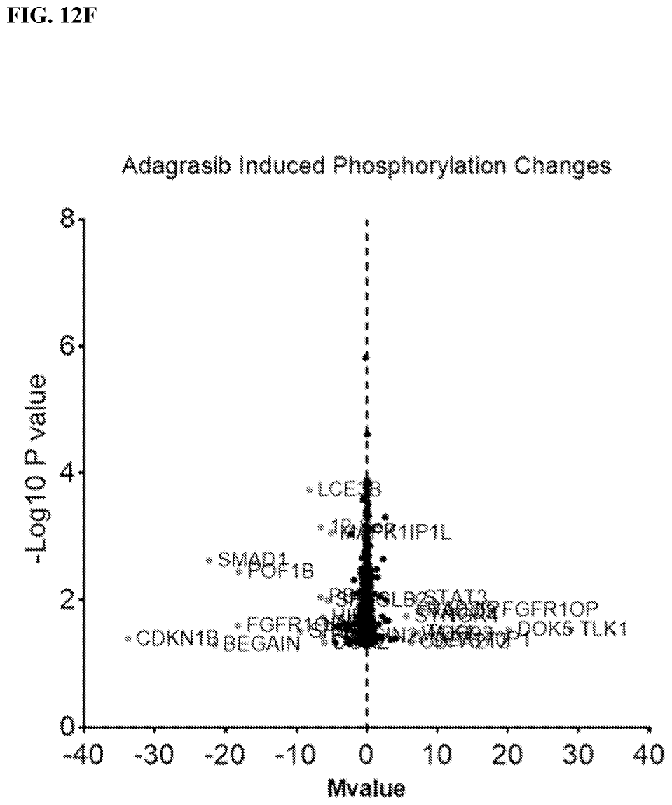

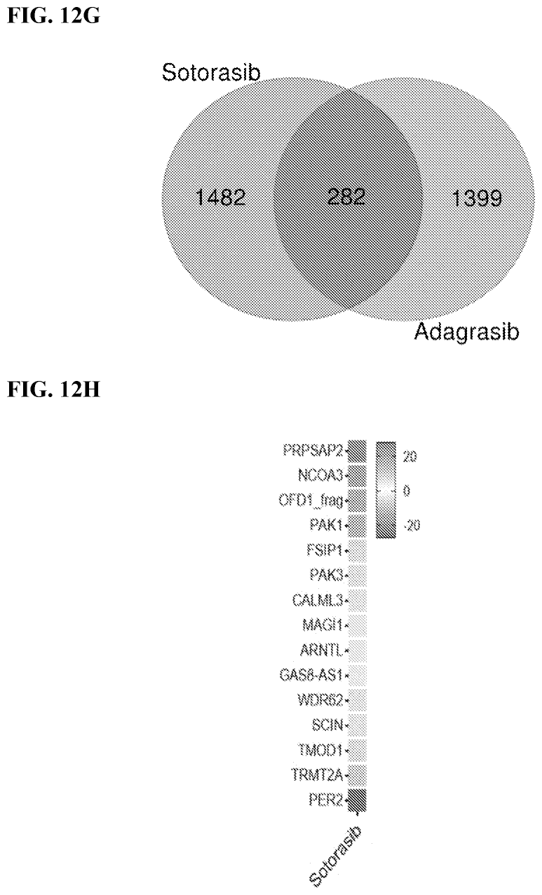

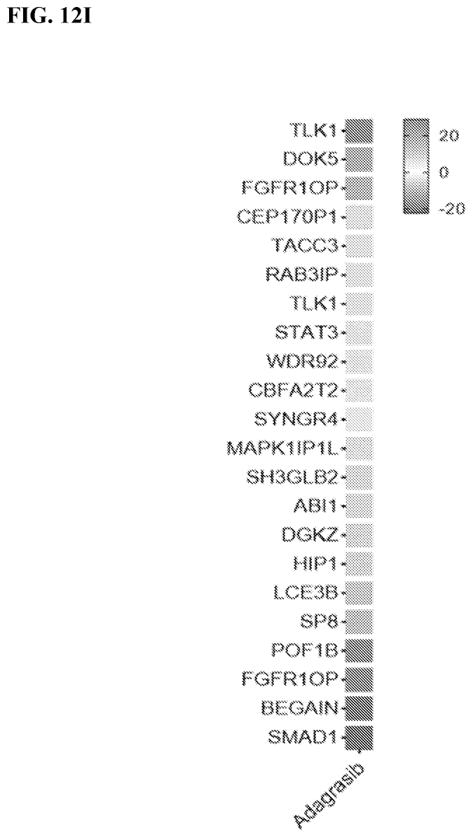

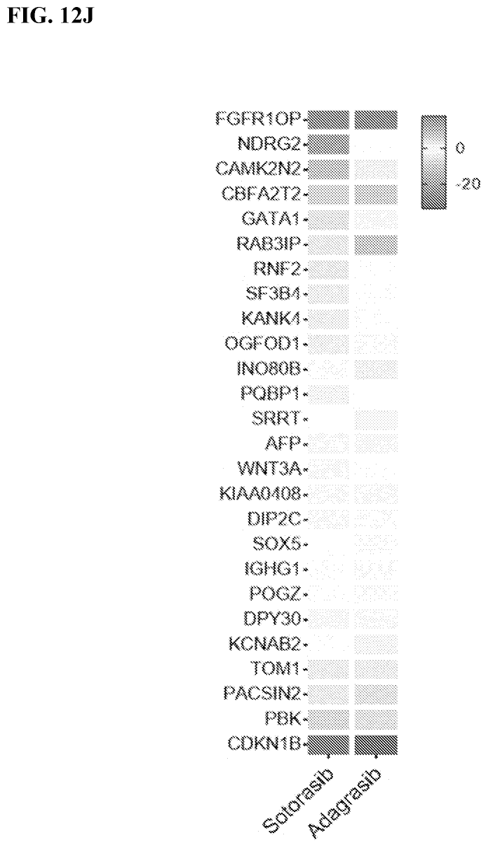

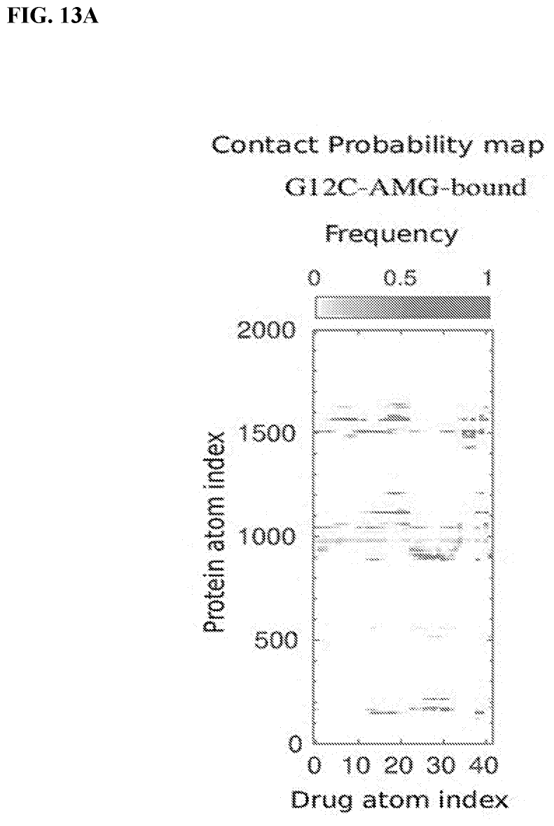

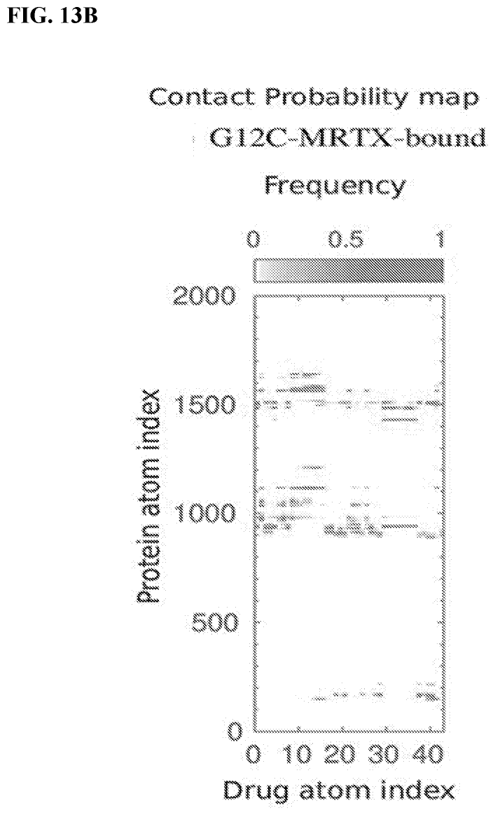

Molecular Dynamic simulations reveal drug-free and drug-bound G12C mutant KRAS have different conformational preferences. Extensive Molecular Dynamics simulations of G12C mutated KRAS in its drug-free state as well as when bound to sotorasib or adagrasib. In FIGS. 12A-12C, it can be seen that in the drug-bound state, the docking site of both the drugs is between the groove created by the P-loop, Switch-II and α3-helix. Upon binding, the drugs induce resistance to the fluctuation of the mutant molecule compared to the drug-free ensemble (FIG. 12D). Adagrasib has a higher potency of imposing this resistance, having a RMSD peak at 2.5 Å as compared to the RMSD peak at 3 Å for the sotorasib bound state. This indicates that the potential interaction of adagrasib with KRAS is more robust than that of sotorasib. Thus, the fine tuning of the conformational plasticity of mutant KRAS could impact its ability to interact with partners. The formation of potential protein-drug interactions at an atomistic resolution limit were analyzed through contact probability maps for both Sotorasib (FIG. 13A) and adagrasib (FIG. 13B) where the normalized frequency monitors the lifetime of each contact pair with respect to the total equilibrium timescale. The comparison between the contact probability map for both the drugs showed that, compared to sotorasib, adagrasib forms more stable contacts (more red data points) with KRAS corroborating the marked decrease in conformational flexibility of adagrasib bound KRAS than seen with sotorasib bound KRAS.

Differential phosphorylation effect of adagrasib and sotorasib in SW1573 cells: SW1573 cells with IC50 concentration of sotorasib or adagrasib was treated for 72 hours and harvested the lysate for determining the global phosphorylation changes induced by these two inhibitors. The phosphorylation changes were analyzed employing the AVM BioMed protein array platform. Sotorasib treatment induced significant phosphorylation changes in 1764 proteins whereas adagrasib treatment induced changes in 1401 proteins (FIGS. 12E-12F). Among them, 282 proteins were common for both the treatments, whereas 1482 proteins were unique for sotorasib and 1399 were unique for adagrasib treatment (FIG. 12G). The proteins with M value greater or less than 5 are represented as heat map for sotorasib treatment or adagrasib treatment (FIG. 12H). The analysis indicates that both inhibitors could induce phosphorylation or dephosphorylation of various targets independently. Next, we observed that some of the proteins like NDRG2, GATA1, RNF2, SF3B4, KANK4, OGFOD1, and WNT3A which were hyperphosphorylated upon sotorasib treatment, became dephosphorylated in presence of adagrasib. Similarly, several proteins which were hypophosphorylated by sotorasib but hyperphosphorylated by adagrasib. Among the common targets, FGFR1 and CDKN1B were consistently highly phosphorylated and dephosphorylated, respectively (FIG. 12I). These data indicate that the KRAS inhibitors can inhibit KRAS function and can also suppress the activity of various proteins associated with KRAS signaling leading to a reduction in growth of NSCLC cells driven by KRAS mutation.

Discussion

Sotorasib and adagrasib are extremely promising mutant selective KRAS G12C inhibitors and, not surprisingly, were recently granted accelerated and breakthrough approval, respectively by the United States Food and Drug Administration. However, resistance to these inhibitors, whether innate or acquired, has already emerged as a serious concern emphasizing the importance of an unbiased and in-depth understanding of the resistance mechanism(s). (Refs. 26-31, 35).

Resistance is generally held to primarily arise through random genetic mutations and the subsequent expansion of mutant clones via Darwinian selection. (Refs 36-37). Hence, the phenomenon has predominantly been approached from a reductionist, gene-centric perspective. (Refs 39, 55). However, it is now evident that therapy resistance arises from heterogeneous drug-tolerant persister cells or minimal residual disease both through genetic and nongenetic mechanisms). Thus, epigenetic modifications and protein interaction network rewiring leading to phenotypic switching, can also impact a cancer cell's ability to develop drug resistance. (Refs. 40-46).

At the outset, it is important to note that, despite covalently binding to the mutant cysteine reside at codon 12, in the present study the 2 drugs demonstrated significant differences in their efficacies and the mechanisms by which they impact signaling. Furthermore, also it was showed that in LUAD cells that are homozygous for the G12C KRAS mutation and hence, more resistant to sotorasib than heterozygous cells, were responsive to adagrasib. Similarly, heterozygous cells that developed resistance to sotorasib, were responsive to adagrasib alluding to potential differences in their mechanisms of action.

Conformational dynamics of proteins, especially of intrinsically disordered proteins (IDPs) that occupy hub positions in cellular protein interaction networks, plays a critical role in signaling by virtue of being able to ‘rewire’ the network. (Refs. 47-48). As a signal transducer protein, KRAS plays an important role in various cellular signaling events. It functions as a critical hub in the cell circuitry that transduces activating signals to several cellular signaling pathways, including the mitogen-activated protein kinase (MAPK) pathway in response to an upstream stimulus. (Ref 49). Although, KRAS may not be, strictly speaking, a bona fide IDP, it may be referred to as a ‘hybrid’ protein. The molecule, more specifically, the G domain (residues 1-166) that is regarded as the functional domain, is mostly ordered. But there are several short, disordered regions for example, the P-loop and Switch I and Switch II regions, interdigitated in between the ordered regions that contribute to its conformational preferences. Thus, the KRAS molecule in its apo form exists as a conformational ensemble with considerable flexibility contributed by the disordered regions. (Refs. 14-15). In contrast, upon binding to GDP/GTP, the ensemble is significantly structured; however, the various point mutations bias the conformational preferences of the holoenzyme. Therefore, though functionally the mutant selective inhibitors impair it's GTPase activity (lock it in the unbound state) and render the oncoprotein inactive, they impinge on its malleability and thereby, impact downstream signaling with potentially different clinical outcomes. Of note, the most common mutations (codons 12, 13 and 61), occur in the P-loop (residues 10-14) and Switch II region (residues 58-72), respectively, further underscoring the crucial role of conformational dynamics.

Consistent with this argument, our MDS data revealed that the adagrasib bound mutant KRAS molecule is more rigid than the sotorasib bound molecule and therefore, could potentially impinge on the partners with which they can interact and hence, account for the difference observed in their efficacies and mechanisms of actions underscoring the importance of the GDP bound KRAS which until the new KRAS inhibitors were discovered, was thought to be inactive. Of note, we have consistently seen differences in the molecular weights of the drug-free KRAS molecule, the sotorasib-bound KRAS, and the adagrasib-bound KRAS molecule in SDS gel electrophoresis. It can be inferred that this is due to conformational, and/or pot-translational modifications such as phosphorylation, differences since the two small molecule inhibitors only differ by about 200 Daltons and the observed differences are about 2-3 kilo Daltons (FIG. 3A).

The motivation to discern the efficacy of carfilzomib in alleviating resistance caused by chronic sotorasib treatment was led by a previous serendipitous discovery that carfilzomib is efficacious in alleviating cisplatin resistance in a KRAS mutated LAUD cell line. Although discovered as a proteasome inhibitor, it was suspected that carfilzomib perturbs the interaction between ITGB4 and PXN both of which in turn interact with focal adhesion kinase (FAK) in the focal adhesion complex. Carfilzomib is in fact a tetrapeptide that appears to dock well in the binding pocket of FAT region in the extreme C terminus of FAK. This region overlaps with the PXN binding cite via the PXN LD motifs. Furthermore, the fact that only carfilzomib, but not two other proteasome inhibitors tested, is effective at nanomolar concentrations, point to the specificity of carfilzomib. Thus, it may be surmised that the focal adhesion complex may play a novel and more nuanced role in drug resistance than was previously appreciated.

Taken together, these results highlight the role of non-genetic mechanisms of drug resistance in cancer and call for a closer attention to the differences in interactions of the various drug-bound ensembles of the mutant KRAS molecule. With the stage now set for a new era in the treatment of KRAS G12C mutant NSCLC, the present results not only shed new light on the genetic/non-genetic duality of drug resistance in cancer (29458961), but they also uncover new strategies to treat these patients that have failed chemotherapy or anti-programmed cell death protein 1 (PD-1) or anti-programmed death ligand 1 (PD-L1) therapies. While such patients with homozygous G12C mutations would benefit from adagrasib therapy, heterozygous patients could be treated with sotorasib and if they develop resistance, a combination of sotorasib and carfilzomib would alleviate resistance. Alternatively, homozygous patients with increased expression of activated β-catenin or carrying a β-catenin mutation, can be treated with sotorasib and β-catenin inhibitor combination.

Materials and Methods

Cell lines and reagents: NSCLC cell lines (H23, H358, and SW1573) and colorectal cancer cell lines (SW837 and SW1463) were obtained from American Type Culture Collection (ATCC) (Manassas, Va., USA). NSCLC cell lines were cultured in RPMI 1640 medium (Corning) supplemented with fetal bovine serum (FBS) (10%), L-glutamine (2 mM), penicillin/streptomycin (50 U/ml), sodium pyruvate (1 mM), and sodium bicarbonate (0.075%) at 37° C., 5% CO2. Carfilzomib (CFZ) and sotorasib (AMG510) were purchased from Selleck Chemicals (Houston, Tex., USA). adagrasib was purchased from MedChemExpress (Monmouth Junction, N.J., USA).

Antibodies: Antibodies against ITGB4 (cat #: 4707), FAK (cat #: 3285), γH2AX (cat #: 2577), p27 (cat #: 3686), phospho-Rb (S807/811) (cat #: 8516), phospho-AKT (S473) (cat #: 4060), AKT (cat #: 4685), phospho-Src (Y416) (cat #: 6943), Src (cat #: 2109), phospho-Erk (T202/Y204) (cat #: 4377), Erk (cat #: 4695), phospho-β-catenin (S675) (cat #: 9567), β-catenin (cat #: 8480), phospho-p70 S6K (T389) (cat #: 9234), p70 S6K (cat #: 2708), phospho-GSK-3β (S9) (cat #: 9336), phospho-EGFR (Y1068) (cat #: 3777), and USP1 (cat #: 8033) were purchased from Cell Signaling Technology (Danvers, Mass., USA). KRAS antibody (cat #: LS-C175665-100) was purchased from LifeSpan BioSciences (Seattle, Wash., USA). PXN antibody (cat #: AH00492) was purchased from Invitrogen (Waltham, Mass., USA). WNT2 antibody (cat #: 66656-1-Ig) was purchased from Proteintech (Rosemont, Ill., USA). HDAC1 antibody (cat #: C15410325-10) was purchased from Diagenode. VDAC1 antibody (cat #: sc-390996) was purchased from Santa Cruz Biotechnology (Dallas, Tex., USA). β-actin antibody was purchased from Sigma-Aldrich (cat #: A5441) (St. Louis, Mo., USA).

Immunoblotting: Cells were lysed 1×RIPA buffer on ice. Protein quantification was performed using Bio-Rad, normalized to equal concentrations, and denatured in 1×reducing sample buffer at 95 C for 5 min. Protein samples (10-20 ug) were run on 4-15% Criterion gels (Bio-Rad) and transferred onto nitrocellulose membranes (Bio-Rad). Cell lysates were prepared with 1×RIPA buffer (MilliporeSigma) and denatured in 1×reducing sample buffer at 95° C. for 5 min. Protein samples (15 μg) were run on 4-15% TGX gels (Bio-Rad, Hercules, Calif., USA) and transferred onto nitrocellulose membranes (Bio-Rad). Blots were blocked with 5% non-fat milk in TBS-T for 1 hour at room temperature and probed with primary antibody diluted in 2.5% BSA in TBS-T overnight at 4° C. After three washes with TBS-T, blots were incubated with HRP-conjugated secondary antibodies for 2 hours at room temperature. After three more washes, bands of interest were visualized via chemiluminescence using WesternBright ECL HRP substrate (Advansta, Menlo Park, Calif., USA) and imaged with the ChemiDoc MP imager (Bio-Rad).

Quantitative real-time PCR and RNAseq: Quantitative real-time PCR (qPCR) reactions were performed using TaqMan Universal PCR Master Mix (Thermo Fisher Scientific, Waltham, Mass.) and analyzed by the Quant Studio? Real-time PCR system (Life Technologies, Grand Island, N.Y.). Total RNA isolation and on-column DNase digestion from cells were performed basing on the manufacturer's protocol RNeasy Plus Mini Kit (Qiagen Cat #: 74134). 1 ug of RNA was used to synthesize the cDNA according to the one step cDNA synthesis kit from QuantaBio (Cat #: 101414-106). TaqMan probes were purchased from ThermoFisher (Waltham, Mass.). The mRNA expression was analyzed using multiplex PCR for the gene of interest and GAPDH as a reference using two independent detection dyes FAM probes and VIC probes respectively. Relative mRNA expression was normalized to GAPDH signals and calculated using the delta Ct method.

RNA was extracted from both single and double knockdown H2009 cells 48 h post siRNA transfection, and total RNAseq was performed by the Integrative Genomics Core at City of Hope. RSeQC showed no substantial bias in the coverage of RNAseq reads. A total of 30 million reads were analyzed for each condition.

Cell proliferation and apoptosis assay: Cell proliferation assays were performed using cell lines stably transfected with NucLight Red Lentivirus (Essen BioScience) to accurately visualize and count the nucleus of a single cell. Cells were seeded on a 96-well plate and allowed to adhere for 24 h. Test compounds were added at indicated concentrations. Caspase-3/7 Green Apoptosis Reagent (Essen BioScience) was also added as a green fluorescent indicator of caspase3/7-mediated apoptotic activity. To monitor cell proliferation and apoptosis over time, the plate was placed in the IncuCyte S3 Live Cell Imaging System (Essen BioScience) and images were acquired every 2 hours. Data analysis was generated by the IncuCyte software using a red fluorescence mask to accurately count each cell nucleus and a green fluorescence mask to measure apoptosis over time. 3D spheroid assay 3D spheroid experiments were performed using cell lines stably transfected with NucLight Red Lentivirus (Essen BioScience) to visualize red fluorescence as an indicator of cell viability. Cells were seeded on a 96-well ultra-low attachment plate and allowed to form spheroids overnight. Drug treatment was added as indicated along with Cytotox Green Reagent (Essen BioScience), used as a green fluorescence indicator of cell death due to loss of cell membrane integrity. To monitor cell proliferation and apoptosis over time, the plate was placed in the IncuCyte S3 Live Cell Imaging System (Essen BioScience) and images were acquired every 2 hours. Data analysis was generated by the IncuCyte software using a red fluorescence mask to accurately measure intensity and area of red fluorescence, indicating spheroid viability and a green fluorescence mask, indicating cell death. Cell cycle analysis. The cell lines which do not express the RFP, the bright filed image was taken to measure the phase are and food change in phase area over time was calculated.

Drug Combination Synergy Assay: For combination index (CI) calculation, SW1573 and H23 incured cell lines were seeded in 96-well plate with 5000 cells per well. Three biological replicates (three 96-well plates for each drug combination) were used. For both cell lines, two drug combinations were used sotorasib and carfilzomib, or adagrasib and carfilzomib, or tegatrabetan and sotorasib. The drugs were used in linear dilution series with dilution factor of 2. Sotorasib doses ranged from 0 μM to 64 μM, carfilzomib from 0 ηM-608 ηM or, tegatrabetan from 0 ηM to 340 μM. The plates were read at 72 hours using the IncuCyte Live Cell Analysis System to measure live cells (Incurred object count per well). Further, R package called SynergyFinder (He et al., 2018) was used to find the nature of drug-drug interaction (i.e. if they work in synergy or antagonistically or non-interactively). For this purpose, the drug response matrix is supplied to the mentioned package, which then uses several models namely Highest Single Agent (Berenbaum, 1989), Loewe additivity (Loewe, 1953), Bliss independence (Bliss, 1939) and Zero Interaction Potency (Yadav et al., 2015) to quantify the degree of drug synergy. The dose response matrix was used to calculate individual CI values for IC₃₀, IC₅₀ and IC₇₀ drug treatments. The output values were used to plot isobolograms using the following formula. CI=IC50 (A) pair IC50 (A)+IC50 (B) pair IC50 (B)

Statistical analysis One-way ANOVA, non-linear regression or simple T-test were performed to calculate significance between data sets as indicated with each result or figure legend. A level of significance of p<0.05 was chosen. Data are presented as mean with standard deviation of the mean (±STD) in all figures in which error bars are shown. Graphs were generated using GraphPad Prism 7 software. Changes in tumor size and body weight during the course of the experiments were visualized by scatter plot.

Immunofluorescence

Zebrafish Xenotransplant Experiments

Zebrafish xenotransplant experiments: NSCLC cell line H23 (KRAS G12C heterozygous) were seeded in a 6-well plate until 60-70% confluency. One day prior to microinjection H23 cells were stained with DiI (fluorescent lipophilic cationic indocarbocyanine) green dye. On the day of microinjection, the 48-hpf (hours post fertilization) zebrafish larvae were dechorionated to release the larvae. The larvae were anesthetized using tricaine (MS-222) at a final concentration of 200 μg/ml (stock 5 mg/ml). The larvae were left in anesthetic for 1-2 h until they were motionless for efficient microinjection. The cells were trypsinized and cell number counted using a cell counter (Nexcelom Bioscience Cellometer Auto T4). The cells were made into a homogenous suspension with 10 cells per nanoliter (n1). The cells were injected in the perivitelline space (PVS) of anesthetized 48-hpf (hours post fertilization) larvae (184 nl=approximately 184 cells) using Nanoject-III manual programmable nanoliter injector. The 24-hpi (hours post injection) zebrafish xenografts were screened for formation of an obvious bolus of cancer cells (tumor) using a fluorescence microscope. The larvae were distributed in a 96-well plate with different treatment sets (untreated and drug treated-sotorasib, adagrasib, and carfilzomib single treatment and combination treatment with carfilzomib). Drug toxicity effects on growth and development were also assessed by examining the length and shape of the zebrafish body. For the untreated sample set, the larvae were left in embryo media throughout the experiment. The larvae were imaged using Zeiss Observer 7 microscope (5× objective) for Day 1 and Day 3 of microinjection. The images were processed using FIJI imaging software. Percent inhibition of tumor growth was calculated. The fluorescent intensity was measured using FIJI image software by thresholding the green channel using Otsu setting. The intensity of Day 1 and Day 3 was compared to calculate inhibition percentage. The error bars represent (±SD), n=10 Untreated larvae and n=15 treated larvae were used for each condition.

Mouse xenograft studies. Athymic nude nu/nu mice were obtained from Charles River, Wilmington, Mass., and were acclimated for a week before beginning the experiment. All animal experiments were carried out in accordance with a protocol approved by the Institutional Animal Care and Use Committee (IACUC #16004). Forty 8-week-old mice were divided into eight groups of 5 animals (treatment with: i) corn oil i.e. vehicle control, ii) sotorasib 10 mg/kg b.w., iii) carfilzomib 2 mg/kg b.w., iv) adagrasib 10 mg/kg b.w., v) BI-3406 10 mg/kg b.w., vi) sotorasib+CFZ, vii) adagrasib+CFZ, and viii) BI-3406+CFZ). All 40 animals were injected with 1×106 SW1573 cells suspensions in 100 μl of PBS, subcutaneously into one flank of each mouse. At the same time, animals were randomized into treatment groups. Treatment was started 12 days after the SW1573 NSCLC cells implantation to see palpable tumor growth. Treatments were given to mice via oral gavage twice a week for 8 weeks. Animals were examined daily for signs of tumor growth. Tumors were measured in two dimensions using calipers and body weights were recorded. Photographs of animals were taken at day 1, day 7, day 14, day 21, day 28, day 35, day 42, day 49, and day 56 after treatment. Photographs of tumors were also taken at day 56 after treatment. At the end of the study, the mice were euthanized by CO₂ asphyxiation followed by cervical dislocation. The tumor weights were compared between groups using an unpaired Student's t-test. A portion of the tumor was fixed in 10% buffered formaldehyde solution and paraffin-embedded for immuno-staining or was snap-frozen in liquid nitrogen for further molecular analysis such as western blotting.

Ethics statement. No human subjects were involved in the present study. All animal studies were conducted according to a protocol approved by the City of Hope Animal Care and Ethics Committee (IACUC protocol #16004). Any mice showing signs of distress, pain, or suffering due to tumor burden were humanely euthanized.

Cell cycle analysis. SW1573 cells infected with the Incucyte® Cell Cycle Green/Red Lentivirus exhibit red fluorescence in G1 and green fluorescence in S/G2/M (A). The stable cell line was generated using the puromycin selection media. Yellow cells are indicative of cell cycle transition from G1 to S while non-fluorescent cells are moving from M to G1. These stable cell lines were treated with the increasing concentration of sotorasib or adagrasib for 72 h and the cells were harvested and FACS analysed to determine the distribution of cells in various stages of cell cycle.

Molecular Dynamics Simulations

System Preparation: The initial configuration of the K-RAS protein used for the G12C mutant simulation was obtained from the protein data bank (PDB ID: 4OBE) from an X-Ray Diffraction study (Ref 55). The glycine at position 12 was then mutated in-silico to cysteine using the mutagenesis tool of PyMol. In addition to this, to compare with wild-type GTP-bound KRAS, a GTP was aligned in place of the GDP using the sequence and structural alignment in PyMol, with an X-Ray Diffraction structure by (WT-KRAS in complex with GTP, PDB ID: 5VQ2). (Ref: 56). Furthermore, for studying the structure with the bound drug sotorasib and adagrasib, X-Ray Diffraction structure from the protein data bank by (PDB ID: 601M) and (PDB ID: 6UTO) were used, respectively. (Refs. 25, 57). Both these structures were homology modelled using the SWISS-MODEL server for accounting for the missing segments. (Ref 58). These three simulations were run using the GROMACS package, with the topologies being generated using the CHARMM36IDPSFF force-field designed explicitly for the proteins with Intrinsically Disordered Regions. (Ref 59). However, since the drug molecules were not present in the original CHARMM36IDPSFF force field, the parameters for those were derived using the SWISS-PARAM module. All of these were then separately centered in dodecahedral boxes and solvated using the TIP3P water model. (Ref 60). The systems were then neutralized using sodium and chloride ions to imitate the natural physiological environment.

Simulation Protocol: After the system preparation, all the systems were energy minimized using the steepest descent algorithm to remove steric clashes. Initially, the protein and bound ligands were position restrained with a force constant of 1000 kcal/mol/nm2 and the solvent was equilibrated. At this point, the systems were allowed to undergo an NVT equilibration maintained at 300 K using the modified Berendsen thermostat (Berendsen et al, 1987) for 6 ns. An NPT equilibration of 7 ns was then performed using the Parrinello-Rahman barostat to maintain an average pressure of 1 bar on all three systems. (Ref 62). A time step of 1 fs was maintained throughout all the simulations, and the leap-frog integrator was used. In the final simulation, the position restraints were removed, and it was extended for 815 ns with NPT parameters. For electrostatic calculations, Particle Mesh Ewald was used with a cubic interpolation of order 4, and grid spacing of 0.16 for Fast Fourier Transform was used. Periodic boundary conditions were used throughout all the simulations in all directions. The neighbor list was updated using a grid system after every 10 steps with a short-range neighbor list cut-off of 1 nm. All the bonds were LINCS-constrained.

Contact Probability Map: To evaluate the contact probability map, first the radial distribution function (RDF) for both the drug-bound form were calculated to define a contact cut-off between the nearest neighbor atom-atom contact pair. The cut-off distance between each drug and the globular protein was determined as 5.6 Å. This cut-off was then used to construct the frequency-dependent contact map plots for both the drug-bound states. For the complete trajectory, a matrix with the number of occurrences for each specific contact was calculated and normalized using the min-max normalization:

$${C}_{i}^{′} = \frac{{C}_{i} - min ( C )}{max ( C ) - min ( C )} = \frac{{C}_{i}}{max ( C )}$$

Since min\left(C\right)=0. Hence, the point with a value of 1.00 signifies the set of atoms in the protein and ligand which stay mostly in contact throughout the simulation.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments described herein may be employed.

### REFERENCES

1. American Cancer Society, Key statistics for lung cancer 2021. https://www.cancer.org/cancer/lung-cancer/about/key-statistics html
2. Salgia R. Expert Rev Mol Diagn. 2016 July; 16(7):737-49.
3. Herbst et al, Nature. 2018 Jan. 24; 553(7689):446-454.
4. Yang et al, Front Oncol. 2019 Sep. 25; 9:953.
5. Dearden et al, Ann Oncol. 2013 September; 24(9):2371-6.
6. Renaud et al, Br J Cancer. 2016 Jul. 26; 115(3):346-53.
7. Yu et al, J Thorac Oncol. 2015 March; 10(3):431-7.
8. Fu et al, J Cancer. 2019 Jan. 29; 10(4):821-828.
9. Wiesweg et al, Oncogene. 2019 April; 38(16):2953-2966.
10. Li et al, Clin Cancer Res. 2018 Oct. 1; 24(19):4854-4864.
11. Nussinov et al, Cell Mol Life Sci. 2017 September; 74(17):3245-3261.
12. Nussinov et al, Cancer Res. 2018 Feb. 1; 78(3):593-602.
13. Pantsar T. Comput Struct Biotechnol J. 2019 Dec. 26; 18:189-198.
14. Pantsar et al, PLoS Comput Biol. 2018 Sep. 10; 14(9):e1006458.
15. Pantsar T. Sci Rep. 2020 Jul. 20; 10(1):11992.
16. Rulli et al, Ann Oncol. 2015 October; 26(10):2079-84.
17. Garassino et al, Ann Oncol. 2011 January; 22(1):235-237.
18. Park et al, Korean J Intern Med. 2017 May; 32(3):514-522.
19. Renaud et al, Clin Lung Cancer. 2018 November; 19(6):e919-e931.
20. Cortinovis et al, Ecancermedicalscience. 2016 Jun. 23; 10:648.
21. Nagano et al, Curr Cancer Drug Targets. 2019; 19(8):595-630.
22. Nnadi et al, J Chem Inf Model. 2018 Feb. 26; 58(2):464-471.
23. Skoulidis et al, N Engl J Med. 2021 Jun. 24; 384(25):2371-2381.
24. Hong et al, N Engl J Med. 2020 Sep. 24; 383(13):1207-1217.
25. Canon et al, Nature. 2019 November; 575(7781):217-223.
26. Awad et al, N Engl J Med. 2021 Jun. 24; 384(25):2382-2393.
27. Dunnett-Kane V, Nicola P, Blackhall F, Lindsay C. Cancers (Basel). 2021 Jan. 5; 13(1):151.
28. Tanaka et al, Cancer Discov. 2021 Apr. 6. doi: 10.1158/2159-8290
29. Koga et al, J Thorac Oncol. 2021 May 7:S1556-0864(21)02132-8. doi: 10.1016/j.jtho.2021.04.015.
30. Addeo et al, Cancers (Basel). 2021 May 21; 13(11):2541.
31. Reck et al, Ann Oncol. 2021 Jun. 2:S0923-7534(21)02045-7.
32. Mohanty et al, iScience. 2020 Aug. 22; 23(9):101496.
33. Akin et al, Oncogene. 2009 May 28; 28(21):2163-72.
34. Wang et al, Curr Oncol Rep. 2021 Feb. 13; 23(3):28
35. Romero, Nat Rev Clin Oncol. 2021 Jul. 15.
36. Alvarez-Arenas et al, Sci Rep. 2019 Jun. 27; 9(1):9332.
37. Greaves et al, Nature. 2012 Jan. 18; 481(7381):306-13.
38. Vogelstein et al, Science. 2013 Mar. 29; 339(6127):1546-58.
39. Salgia and Kulkarni, Trends Cancer. 2018 February; 4(2):110-118.
40. Mahmoudabadi et al, Cell Cycle. 2013 Jan. 1; 12(1):26-31.
41. Kulkarni et al, Mol Cancer. 2013 Sep. 24; 12(1):108.
42. Dawson et al, Cell. 2012 Jul. 6; 150(1):12-27.
43. Jones et al, Nat Rev Genet. 2016 Sep. 15; 17(10):630-641
44. Bell et al, Br J Cancer. 2020 February; 122(4):465-472.
45. Pisco et al, Nat Commun. 2013; 4:2467.
46. Jolly et al, Front Oncol. 2018 Mar. 6; 8:50.
47. Mosca et al, Mol Cell Proteomics. 2012 July; 11(7):M111.014969.
48. Buljan et al, Mol Cell. 2012 Jun. 29; 46(6):871-83
49. Cox et al, Small GTPases. 2010 July; 1(1):2-27.
50. Lou et al, Sci Signal. 2019 May 28; 12(583):eaaw9450
51. Xue et al, Nature. 2020 January; 577(7790):421-425.
52. Mohanty et al, www.biorxiv.org/content/10.1101/781807v1 (2019).
53. Marin-Bejar et al, Cancer Cell. 2021 Jun. 7:S1535-6108(21)00281-6.
54. Hunter et al, Proc Natl Acad Sci USA. 2014 Jun. 17; 111(24):8895-90
55. Xu et al, Acta Crystallogr D Struct Biol. 2017 Dec. 1; 73(Pt 12):970-984.
56. Fell et al., J Med Chem. 2020 Jul. 9; 63(13):6679-6693.
57. Waterhouse et al, Nucleic Acids Res. 2018 Jul. 2; 46(W1):W296-W303.
58. Liu et al, Chem Biol Drug Des. 2018 October; 92(4):1722-1735.
59. Mark et al, Journal of Physical Chemistry A, 2001, 105, 9954-9960.
60. Berendsen et al, J Phys Chem-Us, 1987, 91, 6269-6271.
61. Parrinello et al, J Appl Phys, 1981, 52, 7182-7190.

What is claimed is:

1. A method of treating cancer in a subject in need thereof, the method comprising administering to the subject an effective amount of an ITGB4/PXN pathway inhibitor and an effective amount of a KRAS pathway inhibitor; wherein the cancer has a KRAS mutation.

2. The method of claim 1, further comprising administering to the subject an effective amount of a Wnt/β-catenin pathway inhibitor.

3. A method of treating cancer in a subject in need thereof, the method comprising administering to the subject an effective amount of a Wnt/β-catenin pathway inhibitor and an effective amount of a KRAS pathway inhibitor; wherein the cancer has a KRAS mutation.

4. The method of claim 1, wherein the subject is resistant to treatment with a KRAS pathway inhibitor.

5. A method to select a subject with a KRAS mutation for cancer treatment, the method comprising measuring a KRAS mutation in a biological sample obtained from the subject; wherein if the KRAS mutation is present in the biological sample, the subject is selected for treatment with: (i) an ITGB4/PXN pathway inhibitor and a KRAS pathway inhibitor; (ii) a Wnt/β-catenin pathway inhibitor and a KRAS pathway inhibitor; or (iii) an ITGB4/PXN pathway inhibitor, a Wnt/β-catenin pathway inhibitor, and a KRAS pathway inhibitor.

6. The method of claim 5, wherein the subject is selected for treatment with the ITGB4/PXN pathway inhibitor and the KRAS pathway inhibitor.

7. The method of claim 5, wherein the subject is selected for treatment with the Wnt/β-catenin pathway inhibitor and a KRAS pathway inhibitor.

8. The method of claim 5, wherein the subject is selected for treatment with the ITGB4/PXN pathway inhibitor, the Wnt/β-catenin pathway inhibitor, and the KRAS pathway inhibitor.

9. A method of detecting resistance to cancer treatment with a KRAS pathway inhibitor in a subject in need thereof, the method comprising:
(i) measuring the expression level of ITGB4, PXN, or both in a biological sample obtained from the subject;
(ii) comparing the expression level of ITGB4, PXN, or both in the biological sample obtained from the subject to the expression level of ITGB4, PXN, or both in a control;
wherein an elevated expression level of ITGB4, PXN, or both indicates the subject is resistant to treatment with the KRAS pathway inhibitor.

10. The method of claim 9, further comprising administering to the subject an effective amount of an ITGB4/PXN pathway inhibitor and a KRAS pathway inhibitor when the subject has an elevated expression level of ITGB4, PXN, or both.

11. The method of claim 10, further comprising administering to the subject an effective amount of a Wnt/β-catenin pathway inhibitor.

12. The method of claim 9, further comprising administering to the subject an effective amount of a KRAS pathway inhibitor when the subject does not have an elevated expression level of ITGB4, PXN, or both.

13. The method of claim 12, further comprising monitoring the subject for treatment resistance to the KRAS pathway inhibitor.

14. The method of claim 13, further comprising administering to the subject an effective amount of an ITGB4/PXN pathway inhibitor in addition to the KRAS pathway inhibitor when the subject is treatment resistant to the KRAS pathway inhibitor.

15. The method of claim 14, further comprising administering to the subject an effective amount of a Wnt/β-catenin pathway inhibitor.

16. A method of detecting resistance to cancer treatment with a KRAS pathway inhibitor in a subject in need thereof, the method comprising:
(i) measuring the expression level of Wnt, β-catenin, or both in a biological sample obtained from the subject;
(ii) comparing the expression level of Wnt, β-catenin, or both in the biological sample obtained from the subject to the expression level of Wnt, β-catenin, or both in a control;
wherein an elevated expression level of Wnt, β-catenin, or both indicates the subject is resistant to treatment with the KRAS pathway inhibitor.

17. The method of claim 16, further comprising administering to the subject an effective amount of a Wnt/β-catenin pathway inhibitor and a KRAS pathway inhibitor when the subject has an elevated expression level of Wnt, β-catenin, or both.

18. The method of claim 16, further comprising administering to the subject an effective amount of a KRAS pathway inhibitor when the subject does not have an elevated expression level of Wnt, β-catenin, or both.

19. The method of claim 18, further comprising monitoring the subject for treatment resistance to the KRAS pathway inhibitor.

20. The method of claim 19, further comprising administering to the subject an effective amount of a Wnt/β-catenin pathway inhibitor in addition to the KRAS pathway inhibitor when the subject is treatment resistant to the KRAS pathway inhibitor.

21. The method of claim 1, wherein the KRAS mutation is heterozygous.

22. The method of claim 1, wherein the KRAS mutation is homozygous.

23. A method to treat cancer in a subject in need thereof, the method comprising:
(i) identifying a homozygous KRAS mutation in a biological sample obtained from the subject; and
(ii) administering to the subject having the homozygous KRAS mutation:
(a) an ITGB4/PXN pathway inhibitor and a KRAS pathway inhibitor;
(b) a Wnt/β-catenin pathway inhibitor and a KRAS pathway inhibitor; or
(c) an ITGB4/PXN pathway inhibitor, a Wnt/β-catenin pathway inhibitor, and a KRAS pathway inhibitor.

24. The method of claim 23, comprising administering the ITGB4/PXN pathway inhibitor and the KRAS pathway inhibitor.

25. The method of claim 23, comprising administering the Wnt/β-catenin pathway inhibitor and a KRAS pathway inhibitor.

26. The method of claim 23, comprising administering the ITGB4/PXN pathway inhibitor, the Wnt/β-catenin pathway inhibitor, and the KRAS pathway inhibitor.

27. A method to treat cancer in a subject in need thereof, the method comprising:
(i) identifying a heterozygous KRAS mutation in a biological sample obtained from a subject; and
(ii) administering a KRAS pathway inhibitor to the subject having the heterozygous KRAS mutation.

28. The method of claim 28, further comprising monitoring the subject for treatment resistance to the KRAS pathway inhibitor.

29. The method of claim 28, further comprising administering to the subject an effective amount of an ITGB4/PXN pathway inhibitor when the subject is treatment resistant to the KRAS pathway inhibitor.

30. The method of claim 28, further comprising administering to the subject an effective amount of a Wnt/β-catenin pathway inhibitor when the subject is treatment resistant to the KRAS pathway inhibitor.

31. The method of claim 28, further comprising administering to the subject an effective amount of an ITGB4/PXN pathway inhibitor and a Wnt/β-catenin pathway inhibitor when the subject is treatment resistant to the KRAS pathway inhibitor.

32. The method of claim 1, wherein the KRAS mutation is a KRAS G12 mutation.

33. The method of claim 32, wherein the KRAS G12 mutation is a KRAS G12C mutation.

34. The method of claim 1, wherein the KRAS mutation is a KRAS G12 mutation, a KRAS G13 mutation, a KRAS H61, or a combination of two or more thereof.

35. The method of claim 1, wherein the KRAS mutation is a KRAS G12C mutation, a KRAS G12A mutation, a KRAS G12V mutation, or a KRAS G12D mutation.

36. The method of claim 1, wherein the ITGB4/PXN pathway inhibitor is carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, or cerdulatinib.

37. The method of claim 36, wherein the ITGB4/PXN pathway inhibitor is carfilzomib, ixazomib, or 7-((4-((3-ethynylphenyl)amino)-7-methoxyquinazolin-6-yl)oxy)-N-hydroxyheptanamide.

38. The method of claim 36, wherein the ITGB4/PXN pathway inhibitor is carfilzomib.

39. The method of claim 2, wherein the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, IC-2, JIB-04, FH535, a docataxel/sulforaphane combination, pyrvinium pamoate, SKL2001, or a combination of two or more thereof.

40. The method of any 39, wherein the Wnt/β-catenin pathway inhibitor is tegatrabetan.

41. The method of claim 1, wherein the KRAS pathway inhibitor is a KRAS G12 inhibitor.

42. The method of claim 41, wherein the KRAS pathway inhibitor is KRAS G12C inhibitor.

43. The method of claim 1, wherein the KRAS pathway inhibitor is a pan-KRAS inhibitor.

44. The method of claim 1, wherein the KRAS pathway inhibitor is a KRAS G12 inhibitor, a KRAS G13 inhibitor, or a combination thereof.

45. The method of claim 1, wherein the KRAS pathway inhibitor is sotorasib.

46. The method of claim 1, wherein the KRAS pathway inhibitor is adagrasib.

47. The method of claim 1, wherein the KRAS pathway inhibitor is BI-1701963 or N-((R)-1-(3-amino-5-(trifluoromethyl)phenyl)ethyl)-7-methoxy-2-methyl-6-(((S)-tetrahydrofuran-3-yl)oxy)quinazolin-4-amine.

48. The method of claim 1, wherein the KRAS pathway inhibitor is sotorasib, adagrasib, MRTX1133, ARS1620, BI-1701963, N-((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl(2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one.

49. The method of claim 1, wherein the ITGB4/PXN pathway inhibitor is carfilzomib and the KRAS pathway inhibitor is sotorasib.

50. The method of claim 1, wherein the ITGB4/PXN pathway inhibitor is carfilzomib and the KRAS pathway inhibitor is adagrasib.

51. The method of claim 1, wherein the cancer is lung cancer.

52. The method of claim 51, wherein the lung cancer is non-small cell lung cancer.

53. The method of claim 1, wherein the cancer is colorectal cancer, colon cancer, or pancreatic cancer.

54. The method of claim 1, wherein the cancer is a solid tumor.

55. The method of claim 1, wherein the cancer is a primary cancer or a metastatic cancer.

56. A method of treating non-small cell lung cancer in a patient in need thereof, the method comprising administering to the subject an effective amount of carfilzomib and an effective amount of sotorasib; wherein the non-small cell lung cancer has a KRAS G12C mutation.

57. A method of treating non-small cell lung cancer in a patient in need thereof, the method comprising administering to the subject an effective amount of carfilzomib and an effective amount of adagrasib; wherein the non-small cell lung cancer has a KRAS G12C mutation.

58. A pharmaceutical composition comprising a ITGB4/PXN pathway inhibitor, a KRAS pathway inhibitor, and a pharmaceutically acceptable carrier.

59. The pharmaceutical composition of claim 58, wherein the ITGB4/PXN pathway inhibitor is carfilzomib, tozasertib, dasatinib, afatinib, dacomitinib, poziotinib, pacritinib, ixazomib, osimertinib, 7-((4-((3-ethynylphenyl)amino)-7-methoxyquinazolin-6-yl)oxy)-N-hydroxyheptanamide, or cerdulatinib.

60. The pharmaceutical composition of claim 59, wherein the ITGB4/PXN pathway inhibitor is carfilzomib, ixazomib, or 7-((4-((3-ethynylphenyl)amino)-7-methoxyquinazolin-6-yl)oxy)-N-hydroxyheptanamide.

61. The pharmaceutical composition of claim 60, wherein the ITGB4/PXN pathway inhibitor is carfilzomib.

62. The pharmaceutical composition of claim 58, further comprising a Wnt/β-catenin pathway inhibitor.

63. A pharmaceutical composition comprising a Wnt/β-catenin pathway inhibitor, a KRAS pathway inhibitor, and a pharmaceutically acceptable carrier.

64. The pharmaceutical composition of claim 62, wherein the Wnt/β-catenin pathway inhibitor is tegatrabetan, LF3, KYA1797K, KY1220, iCRT3, iCRT5, iCRT14, ZINC02092166, NLS-StAx-h, foscenvivint, tabituximab barzuxetan, vantictumab, ipafricept, Fz7-21, salinomycin, FJP, BML-286, XAV939, JW74, JW55, NVP-TNKS656, LZZ-02, SSTC3, WNT974, ETC-159, CGX1321, GNF-6231, ICG001, isoquercitrin, FJ9, IWP-2, IWP-4, calphostin C, DKN-01, CWP291, GNE-781, Wnt-059, endo-IWR-1, niclosamide, ONC201, tri(2-furyl)phosphine, a actinomycin D/telmisartan combination, chelerythrine, IC-2, JIB-04, FH535, a docataxel/sulforaphane combination, pyrvinium pamoate, SKL2001, or a combination of two or more thereof.

65. The pharmaceutical composition of claim 64, wherein the Wnt/β-catenin pathway inhibitor is tegatrabetan.

66. The pharmaceutical composition of claim 58, wherein the KRAS pathway inhibitor is a KRAS G12 inhibitor, a KRAS G13 inhibitor, or a combination thereof.

67. The pharmaceutical composition of claim 58, wherein the KRAS pathway inhibitor is a KRAS G12C inhibitor.

68. The pharmaceutical composition of claim 58, wherein the KRAS pathway inhibitor is a pan-KRAS inhibitor.

69. The pharmaceutical composition of claim 58, wherein the KRAS pathway inhibitor is sotorasib.

70. The pharmaceutical composition of claim 58, wherein the KRAS pathway inhibitor is adagrasib.

71. The pharmaceutical composition of claim 58, wherein the KRAS pathway inhibitor is BI-1701963 or N-((R)-1-(3-amino-5-(trifluoromethyl)phenyl)ethyl)-7-methoxy-2-methyl-6-(((S)-tetrahydrofuran-3-yl)oxy)quinazolin-4-amine.

72. The pharmaceutical composition of claim 58, wherein the KRAS pathway inhibitor is sotorasib, adagrasib, MRTX1133, ARS1620, BI-1701963, N-((R)-1-(3-amino-5-(trifluoromethyl)-phenyl)ethyl)-7-methoxy-2-methyl-6-(((S)-tetrahydrofuran-3-yl)oxy)-quinazolin-4-amine, compound 0375-0604, LY3537982, JNJ-74699157, (3S,4S)-8-(6-amino-5-((2-amino-3-chloroyridin-4-yl)thio)pyrazin-2-yl)-3-methyl(2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one.
